(19) United States
(12) Patent Application Publication
(10) Pub. No.: US 2024/0122979 A1
(43) Pub. Date: Apr. 18, 2024

(54) Chimeric Cytokine Receptors

(71) Applicants: Provincial Health Services Authority, Vancouver (CA); UVic Industry Partnerships Inc., Victoria (CA)

(72) Inventors: Brad Nelson, Vancouver (CA); Megan Fuller, Vancouver (CA); Martin J. Boulanger, Victoria (CA)

(21) Appl. No.: 17/767,687

(22) PCT Filed: Oct. 8, 2020
(86) PCT No.: PCT/CA2020/051346

§ 371 Date: Apr. 8, 2022

Related U.S. Application Data
(60) Provisional application No. 62/912,223, filed on Oct. 8, 2019.

(51) Int. Cl. A61K 35/17; A61K 39/00; A61K 45/06; A61P 37/04; C07K 14/715; C12N 5/0783; C12N 15/86

(52) U.S. Cl. CPC A61K 35/17; A61K 39/4611; A61K 39/4631; A61K 39/464418; A61K 39/464419; A61K 45/06; A61P 37/04; C07K 14/7153; C07K 14/7155; C12N 5/0636; C12N 15/86; A61K 2239/21; A61K 2239/22; C12N 2740/15043

(57) ABSTRACT

Described herein are chimeric receptors comprising G-CSFR extracellular domains and the intracellular domains of various multi-subunit cytokine receptors for selective activation of cytokine signaling in cells of interest. In certain aspects, the selective activation of cytokine signaling in cells expressing the chimeric receptors described herein includes the ability to specifically stimulate adoptively transferred cells.

### CROSS REFERENCE TO RELATED APPLICATIONS

The application claims priority to U.S. Provisional Application No. 62/912,223, filed Oct. 8, 2019, which is hereby incorporated by reference in its entirety.

### SEQUENCE LISTING

The instant application contains a Sequence Listing which has been submitted via EFS-Web and is hereby incorporated by reference in its entirety. Said ASCII copy, created on Oct. 7, 2020, is named IKEOO1WO_SL.txt, and is 60,383 bytes in size.

### BACKGROUND OF THE INVENTION

### Field of the Invention

Described herein are chimeric cytokine receptors comprising G-CSFR (Granulocyte-Colony Stimulating Factor Receptor) extracellular domains and the intracellular domains of various multi-subunit cytokine receptors for selective activation of cytokine signaling in cells of interest. Specifically, the present disclosure describes new chimeric cytokine receptors that include multi-subunit intracellular signaling domains to deliver specific cytokine-like signals to cells of interest. The present disclosure also comprises methods, cells and kits for use in adoptive cell transfer (ACT), comprising cells expressing the chimeric cytokine receptors and/or expression vectors encoding chimeric cytokine receptors and/or cytokines that bind the chimeric cytokine receptors.

### Description of the Related Art

Patients who receive cell-based immunotherapy treatments often receive cytokine therapy in the form of interleukin-2 (IL-2). IL-2 therapy is beneficial to these patients because it provides signals for proliferation, viability and effector function to the adoptively transferred immune cells (e.g., T lymphocytes or NK lymphocytes), which improves their efficacy. However, patients who receive IL-2 therapy can experience significant and serious toxicities due to the effects of IL-2 on host immune cells. Therefore, there is a clinical need for variant cytokine receptors and methods of producing cells that express variant cytokine receptors that will be able to specifically undergo activation, proliferation and other immune functions in response to an administered cytokine to reduce or eliminate dose-limiting toxicities and reduce or eliminate the need for lympho-depleting chemotherapy prior to immune cell infusion.

### SUMMARY OF THE INVENTION

In certain embodiments, described herein are chimeric receptors, comprising:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor) and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the at least one signaling molecule binding site is selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain.

In certain aspects, described herein are chimeric receptors, comprising: an ECD of a G-CSFR operatively linked to a second domain; the second domain comprising:
(i)
   (a) a transmembrane domain of gp130;
   (b) a Box 1 and a Box 2 region of gp130; and
   (c) a C-terminal region of IL-2Rβ; or
(ii)
   (a) a transmembrane domain of G-CSFR;
   (b) a Box 1 and a Box 2 region of G-CSFR; and
   (c) a C-terminal region of IL-2Rβ; or
(iii)
   (a) a transmembrane domain of G-CSFR;
   (b) a Box 1 and a Box 2 region of G-CSFR; and
   (c) a C-terminal region of IL-12Rβ₂; or
(iv)
   (a) a transmembrane domain of G-CSFR;
   (b) a Box 1 and a Box 2 region of G-CSFR; and
   (c) a C-terminal region of IL-21R; or
(v)
   (a) a transmembrane domain of IL-2Rβ+γc;
   (b) a Box 1 and a Box 2 region of IL-2Rβ+γc; and
   (c) a C-terminal region of IL-2Rβ+γc; or
(vi)
   (a) a transmembrane domain of G-CSFR
   (b) a Box 1 and a Box 2 region of G-CSFR; and
   (c) a C-terminal region of IL-7Rα.

In certain embodiments, the activated chimeric receptor forms a homodimer, and, optionally, the activation of the chimeric receptor causes a cellular response selected from the group consisting of proliferation, viability and enhanced activity of a cell expressing the chimeric receptor, and, optionally, the chimeric receptor is activated upon contact with a G-CSF, and, optionally, the G-CSF is a wild-type G-CSF, and, optionally, the extracellular domain of the G-CSFR is a wild-type extracellular domain. In certain embodiments, the activated chimeric receptor forms a homodimer, and, optionally, the activation of the chimeric receptor causes a cellular response selected from the group consisting of proliferation, viability and enhanced activity of a cell expressing the chimeric receptor, and, optionally, the chimeric receptor is activated upon contact with a G-CSF, and, optionally, the G-CSF is a wild-type G-CSF, and, optionally, the extracellular domain of the G-CSFR is a wild-type extracellular domain.

In certain embodiments, the chimeric receptor is expressed in a cell, and, optionally, an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, the cell is a stem cell, and, optionally, and optionally, the cell is a primary cell, and, optionally, the cell is a human cell.

In certain embodiments, the ICD comprises: (a) at least a portion of an ICD of IL-2Rβ having an amino acid sequence of SEQ ID NO. 16, 19, 21, 29, 31, 33, 35, 37, or 39; or (b) at least a portion of an ICD of IL-7Rα having an amino acid sequence of SEQ ID NO. 41; or (c) at least a portion of an ICD of IL-21R having an amino acid sequence of SEQ ID NO. 25 or 27; or (d) at least a portion of an ICD of IL-12Rβ₂ having an amino acid sequence of SEQ ID NO. 23, 32 or 26; or (e) at least a portion of an ICD of G-CSFR having an amino acid sequence of SEQ ID NO. 20, 22, 24, 26, 28, 30, 34, 40 or 42; or (f) at least a portion of an ICD of gp130 having an amino acid sequence of SEQ ID NO. 18 or 38; or (g) at least a portion of an ICD of IL-7R having an amino acid sequence of SEQ ID NO. 43; or (h) at least a portion of an ICD of IL-2RG having an amino acid sequence of SEQ ID NO. 17.

In certain embodiments, the transmembrane domain comprises a sequence set forth by:
(a) SEQ ID NO. 8; or (b) SEQ ID NO. 9; or (c) SEQ ID NO. 10; or (d) SEQ ID NO. 11.

In certain aspects, described herein is a nucleic acid encoding a chimeric receptor; wherein the chimeric receptor comprises: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ; and, optionally, the transmembrane domain is a wild-type transmembrane domain. In certain embodiments, the ECD of the G-CSFR is encoded by nucleic acid sequence set forth in SEQ ID NO. 5 or 6. In certain embodiments, the nucleic acid comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

In certain embodiments, the present disclosure describes an expression vector comprising a nucleic acid encoding a chimeric receptor described herein. In certain embodiments, the vector is selected from the group consisting of: a retroviral vector, a lentiviral vector, an adenoviral vector and a plasmid.

In certain aspects, described herein is a nucleic acid encoding a chimeric receptor; wherein the chimeric receptor comprises: an ECD of a G-CSFR operatively linked to a second domain; the second domain comprising:
(i)
   (a) a transmembrane domain of gp130;
   (b) a Box 1 and a Box 2 region of gp130; and
   (c) a C-terminal region of IL-2Rβ; or
(ii)
   (a) a transmembrane domain of G-CSFR;
   (b) a Box 1 and a Box 2 region of G-CSFR; and
   (c) a C-terminal region of IL-2Rβ; or
(iii)
   (a) a transmembrane domain of G-CSFR;
   (b) a Box 1 and a Box 2 region of G-CSFR; and
   (c) a C-terminal region of IL-12Rβ₂; or
(iv)
   (a) a transmembrane domain of G-CSFR;
   (b) a Box 1 and a Box 2 region of G-CSFR; and
   (c) a C-terminal region of IL-21R; or
(v)
   (a) a transmembrane domain of IL-2Rβ+γc;
   (b) a Box 1 and a Box 2 region of IL-2Rβ+γc; and
   (c) a C-terminal region of IL-2Rβ+γc; or
(vi)
   (a) a transmembrane domain of G-CSFR
   (b) a Box 1 and a Box 2 region of G-CSFR; and
   (c) a C-terminal region of IL-7Rα.

In certain embodiments, the ECD of the G-CSFR is encoded by nucleic acid sequence set forth in SEQ ID NO. 5 or 6. In certain embodiments, the nucleic acid comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

In certain aspects, described herein are expression vectors comprising a nucleic acid described herein. In certain embodiments, the vector is selected from the group consisting of: a retroviral vector, a lentiviral vector, an adenoviral vector and a plasmid.

In certain aspects, described herein is a cell comprising a nucleic acid encoding a chimeric receptor; wherein the chimeric receptor comprises:
(a) an extracellular domain (ECD) of a G-CSFR (Granulocyte-Colony Stimulating Factor Receptor) operatively linked Receptor) operatively linked to a second domain; the second domain comprising
(b) at least a portion of an intracellular domain (ICD) of a multi-subunit cytokine receptor selected from the group consisting of: TL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain; and, optionally, the cell is an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain aspects, described herein is a cell comprising a nucleic acid encoding a chimeric receptor; wherein the chimeric receptor comprises: an ECD of a G-CSFR operatively linked to a second domain; the second domain comprising:
(i)
   (a) a transmembrane domain of gp130;
   (b) a Box 1 and a Box 2 region of gp130; and
   (c) a C-terminal region of IL-2Rβ; or
(ii)
   (a) a transmembrane domain of G-CSFR;
   (b) a Box 1 and a Box 2 region of G-CSFR; and
   (c) a C-terminal region of IL-2Rβ; or
(iii)
   (a) a transmembrane domain of G-CSFR;
   (b) a Box 1 and a Box 2 region of G-CSFR; and
   (c) a C-terminal region of IL-12Rβ₂; or
(iv)
   (a) a transmembrane domain of G-CSFR;
   (b) a Box 1 and a Box 2 region of G-CSFR; and
   (c) a C-terminal region of IL-21R; or
(v)
   (a) a transmembrane domain of IL-2Rβ+γc;
   (b) a Box 1 and a Box 2 region of IL-2Rβ+γc; and
   (c) a C-terminal region of IL-2Rβ+γc; or
(vi)
   (a) a transmembrane domain of G-CSFR
   (b) a Box 1 and a Box 2 region of G-CSFR; and
   (c) a C-terminal region of IL-7Rα; and, optionally,
the cell is an immune cell; and, optionally, and, optionally, a T cell, and, optionally, a NK cell, and, optionally, a NKT cell, and, optionally, a B cell, and, optionally, a plasma cell, and optionally, a macrophage, and optionally, a dendritic cell, the cell is a stem cell, and, optionally, and optionally, the cell is a primary cell, and, optionally, the cell is a human cell.

In certain embodiments, the ECD of the G-CSFR is encoded by nucleic acid comprised in the cell has a sequence set forth in SEQ ID NO. 5 or 6. In certain embodiments, the nucleic acid comprised by the cell comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

In certain aspects, described herein is a cell comprising an expression vector described herein, and, optionally, the cell is an immune cell, and, optionally, a T cell or a NK cell. In certain aspects, described herein is a cell comprising the chimeric receptor of claim 1, and, optionally, the cell in an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and, optionally, the cell is a primary cell, and, optionally, the cell is a human cell. In certain aspects, described herein is a cell comprising the chimeric receptor described herein, and, optionally, the cell in an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain aspects, described herein is a method of selective activation of a chimeric receptor expressed on the surface of a cell, comprising: contacting a chimeric receptor with a G-CSF that selectively activates the chimeric receptor; wherein the chimeric receptor comprises: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R, and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain.

In certain aspects, described herein is a method of selective activation of a chimeric receptor expressed on the surface of a cell, comprising: contacting a chimeric receptor with a G-CSF that selectively activates the chimeric receptor; wherein the chimeric receptor, comprises an ECD of a G-CSFR operatively linked to a second domain; the second domain comprising:
(i)
   (a) a transmembrane domain of gp130;
   (b) a Box 1 and a Box 2 region of gp130; and
   (c) a C-terminal region of IL-2Rβ; or
(ii)
   (a) a transmembrane domain of G-CSFR;
   (b) a Box 1 and a Box 2 region of G-CSFR; and
   (c) a C-terminal region of IL-2Rβ; or
(iii)
   (a) a transmembrane domain of G-CSFR;
   (b) a Box 1 and a Box 2 region of G-CSFR; and
   (c) a C-terminal region of IL-12Rβ₂; or
(iv)
   (a) a transmembrane domain of G-CSFR;
   (b) a Box 1 and a Box 2 region of G-CSFR; and
   (c) a C-terminal region of IL-21R; or
(v)
   (a) a transmembrane domain of IL-2Rβ+γc;
   (b) a Box 1 and a Box 2 region of IL-2Rβ+γc; and
   (c) a C-terminal region of IL-2Rβ+γc; or
(vi)
   (a) a transmembrane domain of G-CSFR
   (b) a Box 1 and a Box 2 region of G-CSFR; and
   (c) a C-terminal region of IL-7Rα.

In certain embodiments of the methods described herein, the activated chimeric receptor forms a homodimer, and, optionally, the activation of the chimeric receptor causes a cellular response selected from the group consisting of proliferation, viability and enhanced activity of a cell expressing the chimeric receptor; and, optionally, the chimeric receptor is activated upon contact with a G-CSF, and, optionally, the G-CSF is a wild-type G-CSF, and, optionally, the extracellular domain of the G-CSFR is a wild-type extracellular domain; wherein the chimeric receptor is expressed in a cell, and, optionally, an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain embodiments of the methods described herein, the chimeric receptor comprises
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or (c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein the transmembrane domain comprises a sequence set forth by: (a) SEQ ID NO. 8; or
(b) SEQ ID NO. 9; or (c) SEQ ID NO. 10; or (d) SEQ ID NO. 11.

In certain aspects, described herein is a method of producing a chimeric receptor in an cell, comprising: introducing into the cell the nucleic acid of any one of claims 13-16, or 19-22 or the expression vector of any one of claims claim 17, 18, 23 or 24; and, optionally, the method comprises gene editing; and, optionally, the cell is an immune cell; and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain embodiments, described herein is a method of treating a subject in need thereof, comprising: infusing into the subject a cell expressing a chimeric receptor and administering a cytokine that binds the chimeric receptor; wherein the chimeric receptor comprises:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor); and, optionally, the IL-2R is selected from the group consisting of IL-2Rβ and IL-2Rγc; and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor; and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7R; a phosphatidylinositol 3-kinase (PI3K) binding site of IL-7R; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises comprising a third domain comprising at least a portion of a transmembrane domain of a protein selected from the group consisting of: G-CSFR, gp130 (Glycoprotein 130), and IL-2Rβ; and, optionally, the transmembrane domain is a wild-type transmembrane domain.

In certain aspects, described herein is a method of treating a subject in need thereof, comprising: infusing into the subject a cell expressing a chimeric receptor and administering a cytokine that binds the chimeric receptor; wherein the chimeric receptor comprises: an ECD of a G-CSFR operatively linked to a second domain; the second domain comprising:
(i)
   (a) a transmembrane domain of gp130;
   (b) a Box 1 and a Box 2 region of gp130; and
   (c) a C-terminal region of IL-2Rβ; or
(ii)
   (a) a transmembrane domain of G-CSFR;
   (b) a Box 1 and a Box 2 region of G-CSFR; and
   (c) a C-terminal region of IL-2Rβ; or
(iii)
   (a) a transmembrane domain of G-CSFR;
   (b) a Box 1 and a Box 2 region of G-CSFR; and
   (c) a C-terminal region of IL-12Rβ₂; or
(iv)
   (a) a transmembrane domain of G-CSFR;
   (b) a Box 1 and a Box 2 region of G-CSFR; and
   (c) a C-terminal region of IL-21R; or
(v)
   (a) a transmembrane domain of IL-2Rβ+γc;
   (b) a Box 1 and a Box 2 region of IL-2Rβ+γc; and
   (c) a C-terminal region of IL-2Rβ+γc; or
(vi)
   (a) a transmembrane domain of G-CSFR
   (b) a Box 1 and a Box 2 region of G-CSFR; and
   (c) a C-terminal region of IL-7Rα.

In certain embodiments of the method, the activated chimeric receptor forms a homodimer; and, optionally, the activation of the chimeric receptor causes a cellular response selected from the group consisting of proliferation, viability and enhanced activity of a cell expressing the chimeric receptor; and, optionally, the chimeric receptor is activated upon contact with a G-CSF; and, optionally, the G-CSF is a wild-type G-CSF; and, optionally, the extracellular domain of the G-CSFR is a wild-type extracellular domain; wherein the chimeric receptor is expressed in a cell; and, optionally, the cell is an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell. In certain embodiments, the chimeric receptor optionally comprises (a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or (c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein the transmembrane domain comprises a sequence set forth by: (a) SEQ ID NO. 8; or (b) SEQ ID NO. 9; or (c) SEQ ID NO. 10; or (d) SEQ ID NO. 11.

In certain embodiments, the methods described herein are used to treat cancer. In certain embodiments, the method is used to treat an autoimmune disease. In certain embodiments, the method is used to treat an inflammatory condition. In certain embodiments, the method is used to prevent or treat graft rejection. In certain embodiments, the method is used to treat an infectious disease. In certain embodiments, the method further comprises administering at least one additional active agent; and, optionally, the additional active agent is an additional cytokine.

In certain embodiments, the methods described herein comprise: i) isolating an immune cell-containing sample; (ii) transducing or transfecting the immune cells with a nucleic acid sequence encoding the chimeric cytokine receptor; (iii) administering or infusing the immune cells to the subject; and (iv) contacting the immune cells with the cytokine that binds the chimeric receptor. In certain embodiments, the subject has undergone an immuno-depletion treatment prior to administering or infusing the cells to the subject. In certain embodiments, the immune cell-containing sample is isolated from the subject that will be administered or infused with the cells. In certain embodiments, the immune cells are contacted with the cytokine in vitro prior to administering or infusing the cells to the subject. In certain embodiments, the immune cells are contacted with the cytokine that binds the chimeric receptor for a sufficient time to activate signaling from the chimeric receptor.

Described herein is a kit for treating a subject in need thereof, comprising: cells encoding a chimeric receptor described herein, and, optionally, the cells are immune cells; and instructions for use; and, optionally, the kit comprises a cytokine that binds the chimeric receptor. Described herein is a kit for producing a chimeric receptor expressed on a cell, comprising: an expression vector encoding a chimeric receptor described herein and instructions for use; and, optionally, the kit comprises a cytokine that binds the chimeric receptor.

Described herein is a kit for producing a chimeric receptor expressed on a cell, comprising: cells comprising an expression vector encoding a chimeric receptor described herein and, optionally, the cells are bacterial cells, and instructions for use; and, optionally, the kit comprises a cytokine that binds the chimeric receptor.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a schematic of native IL-2Rβ, IL-2Rγc, and G-CSFR subunits, as well as the G2R-1 receptor subunit designs.

FIG. 2 presents graphs showing the expansion (fold change in cell number) of 32D-IL-2Rβ cells (which is the 32D cell line stably expressing the human IL-2Rβ subunit) expressing the indicated G-CSFR chimeric receptor subunits and stimulated with WT G-CSF, IL-2 or no cytokine. G/γc was tagged at its N-terminus with a Myc epitope (Myc/G/γc), and G/IL-2R was tagged at its N-terminus with a Flag epitope (Flag/G/IL-2Rβ); these epitope tags aid detection by flow cytometry and do not impact the function of the receptors. In addition, the lower panels in B-D show the percentage of cells expressing the G-CSFR ECD (% G-CSFR+) under each of the culture conditions. Squares represent cells stimulated with IL-2. Triangles represent cells stimulated with G-CSF. Circles represent cells not stimulated with a cytokine.

FIG. 3 presents graphs showing the expansion (fold change in cell number) of human T cells expressing the Flag-tagged G/IL-2Rβ subunit alone, the Myc-tagged G/γc subunit alone, or the full-length G-CSFR. A-D) PBMC-derived T cells; E-H) tumour-associated lymphocytes (TAL). Squares represent cells stimulated with IL-2. Triangles represent cells stimulated with G-CSF. Circles represent cells not stimulated with cytokine.

FIG. 4 is a schematic of native and chimeric receptors, showing JAK, STAT, Shc, SHP-2 and PI3K binding sites. The shading scheme includes receptors from FIG. 1.

FIG. 5 is a schematic of chimeric receptors, showing Jak, STAT, Shc, SHP-2 and PI3K binding sites. The shading scheme includes receptors from FIGS. 1 and 4.

FIG. 6 is a diagram of the lentiviral plasmid containing the G2R-2 cDNA insert.

FIG. 7 presents graphs showing G-CSFR ECD expression assessed by flow cytometry in cells transduced with G2R-2. A) 32D-IL-2Rβ cell line; B) PBMC-derived human T cells and human tumour-associated lymphocytes (TAL).

FIG. 8 presents graphs showing the expansion (fold change in cell number) of cells expressing G2R-2 compared to non-transduced cells. A) Human PBMC-derived T cells; B, C) Human tumour-associated lymphocytes (TAL) from two independent experiments. Squares represent cells stimulated with IL-2. Triangles represent cells stimulated with G-CSF. Circles represent cells not stimulated with cytokine.

FIG. 9 presents graphs showing expansion (fold change in cell number) of CD4- or CD8-selected human tumour-associated lymphocytes expressing G2R-2 compared to non-transduced cells. A) Non-transduced CD4-selected cells; B) Non-transduced CD8-selected cells; C) CD4-selected cells transduced with G2R-2; D) CD8-selected cells transduced with G2R-2. Dotted gray line represents cells stimulated with IL-2. Solid black line represents cells stimulated with G-CSF. Dashed gray line represents cells not stimulated with cytokine.

FIG. 10 presents graphs showing expansion (fold change in cell number) of CD4+ or CD8+ tumour-associated lymphocytes expressing G2R-2. The cells were initially expanded in G-CSF or IL-2, as indicated. Cells were then plated in either IL-2, G-CSF or medium only. Solid gray line represents cells stimulated with IL-2. Solid black line represents cells stimulated with G-CSF. Dashed light gray line represents cells expanded in IL-2 and then stimulated with medium only. Dashed dark gray line represents cells expanded in G-CSF and then stimulated with medium only.

FIG. 11 presents graphs showing immunophenotype (by flow cytometry) of CD4- or CD8-selected tumour-associated lymphocytes (TAL) expressing G2R-2 chimeric receptor construct versus non-transduced cells, after expansion in G-CSF or IL-2. A) Percentage of live cells showing a CD4+, CD8+ or CD3-CD56+ cell surface phenotype. B) Percentage of live CD8+ cells showing the indicated cell surface phenotypes based on CD45RA and CCR7 expression.

FIG. 12 presents graphs showing the results of BrdU incorporation assays to assess proliferation of primary human T cells expressing G2R-2 versus non-transduced cells. T cells were selected by culture in IL-2 or G-CSF, as indicated, prior to the assay. A) Tumour-associated lymphocytes; B) PBMC-derived T cells.

FIG. 13 presents graphs showing the results of BrdU incorporation assays to assess proliferation of primary murine T cells expressing G2R-2 or the single-chain G/IL-2Rβ (a component of G2R-1) versus mock-transduced cells. A) Transduction efficiency as reflected by the percentage of cells expressing the G-CSFR ECD (by flow cytometry) after culture in the indicated cytokines; B) Percent BrdU incorporation in all live cells in response to the indicated cytokines; C) Percent BrdU incorporation by cells expressing the G-CSFR ECD (G-CSFR+ cells). All cells were expanded in IL-2 for 3 days prior to assay. Squares represent cells stimulated with IL-2. Triangles represent cells stimulated with G-CSF. Circles represent cells not stimulated with a cytokine.

FIG. 14 presents western blots to detect the indicated cytokine signaling events in human primary T cells expressing G2R-2 versus non-transduced cells. β-actin, total Akt and histone H3 serve as a protein loading controls. A, B) Tumour-associated lymphocytes (TALs); C) PBMC-derived T cells.

FIG. 15 presents western blots to detect the indicated cytokine signaling events in primary murine T cells expressing G2R-2 or the single-chain G/IL-2Rβ (from G2R-1) versus mock-transduced cells. Arrow indicates the specific phospho-Jak2 band; other larger bands are presumed to be the result of cross-reactivity of the primary anti-phospho-Jak2 antibody with phospho-Jak1. β-actin and histone H3 serve as protein loading controls.

FIG. 16 is a graph showing the results of a BrdU incorporation assay to assess cell cycle progression of 32D-IL-2Rβ cells expressing the indicated chimeric receptors (or non-transduced cells) in response to stimulation with no cytokine, IL-2 (300 IU/mL), WT G-CSF (30 ng/mL) or 130 G-CSF (30 ng/mL).

FIG. 17 presents graphs showing the results of BrdU incorporation assays to assess cell cycle progression of primary murine T cells expressing the indicated chimeric receptors (or non-transduced cells) in response to stimulation with no cytokine, IL-2 or WT, 130, 304 or 307 cytokine. A and B represent experimental replicates.

FIG. 18 presents western blots to detect the indicated cytokine signaling events in 32D-IL-2Rβ cells expressing the indicated chimeric receptor subunits (or non-transduced cells) in response to stimulation with no cytokine, IL-2, WT G-CSF or 130 G-CSF. β-actin and histone H3 serve as protein loading controls.

FIG. 19 presents A) western blots to detect the indicated cytokine signaling events in primary murine T cells expressing the indicated chimeric receptor subunits in response to stimulation with no cytokine, IL-2, WT G-CSF, 130 G-CSF or 304 G-CSF. β-actin and histone H3 serve as protein loading controls. B) Transduction efficiency of cells used in panel A, as assessed by flow cytometry with an antibody specific for the extracellular domain of the human G-CSF receptor.

FIG. 20 presents plots showing G-CSFR ECD expression by flow cytometry in primary human tumour-associated lymphocytes (TAL) transduced with the indicated chimeric receptor constructs. Live CD3+, CD56− cells were gated on CD8 or CD4, and G-CSFR ECD expression is shown for each population.

FIG. 21 presents graphs and images showing the expansion, proliferation and signaling of primary human tumour-associated lymphocytes (TAL) expressing G2R-3 versus non-transduced cells. A) Graph showing the results of a T-cell expansion assay, where cells were transduced with G2R-3-encoding lentivirus, washed, and re-plated in IL-2 (300 IU/ml), wildtype G-CSF (100 ng/ml) or no cytokine. Live cells were counted every 3-4 days. Squares represent cells stimulated with IL-2. Triangles represent cells stimulated with G-CSF. Circles represent cells not stimulated with a cytokine. B) Western blot to assess intracellular signaling events. Cells were harvested from the expansion assay and stimulated with IL-2 (300 IU/ml) or wildtype G-CSF (100 ng/ml). Arrow indicates the specific phospho-Jak2 band at 125 kDa; larger bands are presumed to be the result of cross-reactivity of the primary anti-phospho-Jak2 antibody with phospho-Jak1. β-actin and histone H3 serve as protein loading controls. C) Graph showing the results of a BrdU incorporation assay to assess T-cell proliferation. Cells were harvested from the expansion assay, washed, and re-plated in IL-2 (300 IU/ml), wildtype G-CSF (100 ng/ml) or no cytokine.

FIG. 22 presents graphs showing the fold expansion and G-CSFR ECD expression of primary human PBMC-derived T cells expressing G2R-3 with WT ECD versus non-transduced cells. A) Graph showing the results of a T-cell expansion assay, where cells were transduced with G2R-3-encoding lentivirus. On Day 1, WT G-CSF (100 ng/ml) or no cytokine (medium alone) were added to the culture. Thereafter, to Day 21, cells were replenished with medium containing WT G-CSF or no cytokine. On Day 21 of expansion, cells were washed and re-plated in WT G-CSF (100 ng/mL), IL-7 (20 ng/mL) and IL-15 (20 ng/mL), or no cytokine. Live cells were counted every 2-4 days. Squares represent cells stimulated with G-CSF. Triangles represent cells stimulated with G-CSF and re-plated in IL-7 and IL-15 on Day 21. Circles represent cells not stimulated with a cytokine. Diamonds represent cells stimulated with G-CSF, and re-plated in medium only on Day 21. B) Graph showing the expression of the G-CSFR ECD, as determined by flow cytometry, on Day 21 or 42 of expansion.

FIG. 23 presents graphs showing the intracellular signaling and immunophenotype of primary human PBMC-derived T cells expressing G2R-3 versus non-transduced cells. A) Western blot to assess intracellular signaling events. Cells were harvested from an expansion assay and stimulated with IL-2 (300 IU/ml) or wildtype G-CSF (100 ng/ml). β-actin serves as protein loading control. B, C) Representative flow cytometry plots and graphs showing the immunophenotype, assessed by flow cytometry, of cells expressing G2R-3 versus non-transduced cells on Day 42 of expansion.

FIG. 24 presents graphs showing the fold expansion of primary human PBMC-derived T cells expressing G2R-3 with 304 or 307 ECD versus non-transduced cells. A) Graph showing the results of a T-cell expansion assay, where cells were transduced with G2R-3 304 ECD-encoding lentivirus. B) Graph showing the results of a T-cell expansion assay, where cells were transduced with G2R-3 307 ECD-encoding lentivirus. C) Graph showing the results of a T-cell expansion assay with non-transduced cells. On Day 2 IL-2 (300 IU/mL), 304 G-CSF (100 ng/ml), 307 G-CSF (100 ng/mL) or no cytokine (medium alone) were added to the culture, as indicated, and replenished every two days thereafter. Live cells were counted every 3-4 days. Diamonds represent cells stimulated with 304 G-CSF. Squares represent cells stimulated with 307 G-CSF. Triangles represent cells stimulated with IL-2. Inverted triangles represent cells not stimulated with a cytokine.

FIG. 25 presents a graph showing the results of a BrdU incorporation assay to assess proliferation of primary human PBMC-derived T cells expressing G2R-3 with 304 or 307 ECD versus non-transduced cells. Cells were transduced with G2R-3 304 ECD- or 307 ECD-encoding lentivirus and expanded in the 304 or 307 G-CSF (100 ng/mL). Non-transduced cells were expanded in IL-2 (300 IU/mL). On Day 12 of expansion cells were washed, and re-plated in IL-2 (300 IU/ml), 130 G-CSF (100 ng/ml), 304 G-CSF (100 ng/ml), 307 G-CSF (100 ng/ml) or no cytokine.

FIG. 26 presents a graph showing G-CSFR ECD expression by flow cytometry in primary murine T cells transduced with the indicated chimeric receptor constructs.

FIG. 27 shows G-CSF-induced phosphorylation of STAT3 (detected by flow cytometry) in primary PBMC-derived human T cells expressing G21R-1 or G21R-2. Cells were subdivided (i.e., gated) into G-CSFR-positive (upper panels) or G-CSFR-negative (lower panels) populations.

FIG. 28 presents graphs and images showing G-CSF-induced biochemical signaling events in primary murine T cells expressing G21R-1 or G12R-1. A) Graph showing phosphorylation of STAT3 (detected by flow cytometry) in CD4+ or CD8+ cells transduced with G21R-1 and stimulated with no cytokine, IL-21 or G-CSF. B) Graph showing the percentage of cells staining positive for phospho-STAT3 after stimulation with no cytokine (black circles), IL-21 (squares) or WT G-CSF (gray circles). Live cells were gated on CD8 or CD4, and the percentage of phospho-STAT3-positive cells is shown for each population. C) Western blots to assess the indicated cytokine signaling events in cells expressing G21R-1 or G12R-1 and stimulated with IL-21, IL-12 or WT G-CSF. β-actin and histone H3 serve as protein loading controls.

FIG. 29 presents graphs and images showing proliferation, G-CSFR ECD expression and WT G-CSF-induced intracellular signaling events in primary murine T cells expressing G2R-2, G2R-3, G7R-1, G21/7R-1 and G27/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are independent experimental replicates. C) Graph showing G-CSFR ECD expression by flow cytometry in primary murine T cells transduced with the indicated chimeric receptor constructs. D) Western blots to assess the indicated cytokine signaling events in cells expressing G2R-2, G2R-3, G7R-1, G21/7R-1 and G27/2R-1, or mock-transduced T cells. Cells were stimulated with IL-2 (300 IU/mL), IL-7 (10 ng/mL), IL-21 (10 ng/mL), IL-27 (50 ng/mL) or G-CSF (100 ng/mL). β-actin and histone H3 serve as protein loading controls. FIG. 30 presents graphs and images showing proliferation, G-CSFR ECD expression and G-CSF-induced biochemical signaling events in primary murine T cells expressing G21/2R-1, G12/2R-1 and 21/12/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are independent experimental replicates. C) Graph showing G-CSFR ECD expression by flow cytometry in primary murine T cells transduced with the indicated chimeric receptor constructs. D) Western blots to assess the indicated cytokine signaling events in cells expressing G21/2R-1, G12/2R-1 and 21/12/2R-1 or mock-transduced T cells. Cells were stimulated with IL-2 (300 IU/mL), IL-21 (10 ng/mL), IL-12 (10 ng/mL) or G-CSF (100 ng/mL). β-actin and histone H3 serve as protein loading controls.

FIG. 30 presents graphs and images showing proliferation, G-CSFR ECD expression and G-CSF-induced biochemical signaling events in primary murine T cells expressing G21/2R-1, G12/2R-1 and 21/12/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are experimental replicates. C) Graph showing G-CSFR ECD expression by flow cytometry in primary murine T cells transduced with the indicated chimeric receptor constructs. D) Western blots to assess the indicated cytokine signaling events in cells expressing G21/2R-1, G12/2R-1 and 21/12/2R-1 or mock-transduced T cells. Cells were stimulated with IL-2 (300 IU/mL), IL-21 (10 ng/mL), IL-12 (10 ng/mL) or G-CSF (100 ng/mL). β-actin and histone H3 serve as protein loading controls.

FIG. 31 presents graphs showing the fold expansion and G-CSFR ECD expression of primary human PBMC-derived T cells expressing G12/2R-1 with 134 ECD, versus non-transduced cells. A) Graph showing the results of a T-cell expansion assay, where cells were transduced with G12/2R-1_134-ECD-encoding lentivirus and expanded in IL-2 (300 IU/mL), 130 G-CSF (100 ng/ml) or medium. Live cells were counted every 4-5 days. Squares represent cells not stimulated with a cytokine. Triangles represent cells stimulated with 130 G-CSF. Diamonds represent cells stimulated with IL-2. B) Graph showing the results of a T-cell expansion assay, where cells were transduced as in panel A. On Day 19 of expansion, cells were washed in re-plated in IL-2, 130 G-CSF or medium only. Live cells were counted every 4-5 days. Squares represent cells not stimulated with a cytokine. Light grey diamonds represent cells stimulated with 130 G-CSF. Dark grey diamonds represent cells stimulated with IL-2. Light grey inverted triangles represent cells initially stimulated with IL-2, and then re-plated in medium only on Day 19. Dark grey triangles represent cells initially stimulated with 130 G-CSF, and then re-plated in medium only on Day 19. C) Graph showing the expression of the G-CSFR ECD, as determined by flow cytometry, on Day 4 or 16 of expansion.

FIG. 32 presents graphs showing the proliferation and immunophenotype of primary human PBMC-derived T cells expressing G12/2R-1 with 134 ECD, versus non-transduced cells. A) Graph showing the results of a BrdU incorporation assay to assess T-cell proliferation. Cells were harvested, washed, and re-plated in IL-2 (300 IU/ml), IL-2+IL-12 (300 IU/ml and 10 ng/mL, respectively), 130 G-CSF (300 ng/ml) or medium alone. B, C) Representative flow cytometry plots and graph showing the immunophenotype, assessed by flow cytometry, of cells expressing G12/2R-1 with 134 ECD, versus non-transduced cells, on Day 16 of expansion.

FIG. 33 presents graphs showing the fold expansion and proliferation of primary human PBMC-derived T cells expressing G12/2R-1 with 304 ECD, versus non-transduced cells. A) Graph showing the results of a T-cell expansion assay, where cells were transduced with G12/2R-1_134-ECD-encoding lentivirus, and expanded in IL-2 (300 IU/mL), 130 G-CSF (100 ng/ml), 304 G-CSF (100 ng/ml) or medium alone. Non-transduced cells were cultured in IL-2, 130 G-CSF, 304 G-CSF or medium alone. Live cells were counted every 3-4 days. Inverted triangles represent cells not stimulated with cytokine. Triangles represent cells stimulated with IL-2. Circles represent cells stimulated with 130 G-CSF. Diamonds represent cells stimulated with 304 G-CSF. B) Cells were harvested from the expansion assay on Day 12, and were washed and re-plated in IL-2 (300 IU/ml), 130 G-CSF (300 ng/ml), 304 G-CSF (100 ng/ml), 307 G-CSF (100 ng/ml) or medium alone.

FIG. 34 presents western blots to detect the indicated cytokine signaling events in primary PBMC-derived T cells expressing G2R-3 with 304 ECD, G12/2R-1 with 304 ECD, or non-transduced T cells. Cells were harvested from the expansion assay and stimulated with 304 G-CSF (100 ng/mL), IL-2 (300 IU/mL), IL-2 and IL-12 (10 ng/mL), or medium alone, as indicated. β-actin and histone H3 serve as protein loading controls.

### DETAILED DESCRIPTION OF THE INVENTION

Briefly, and as described in more detail below, described herein are chimeric receptors comprising G-CSFR extracellular domains and the intracellular domains of various multi-subunit cytokine receptors for selective activation of cytokine signaling in cells of interest. In certain aspects, the selective activation of cytokine signaling in cells expressing the chimeric receptors described herein include the ability to specifically stimulate adoptively transferred cells. Thus, described herein are new processes and compositions of matter that have the potential to improve cell-based therapies for a variety of disease indications.

### Definitions

Terms used in the claims and specification are defined as set forth below unless otherwise specified.

The term “treatment” refers to any therapeutically beneficial result in the treatment of a disease state, e.g., a cancer disease state, including prophylaxis, lessening in the severity or progression, remission, or cure thereof.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to selectively activate a receptor expressed on a cell.

The term “therapeutically effective amount” is an amount that is effective to ameliorate a symptom of a disease.

The term “wild-type” refers to the native amino acid sequence of a polypeptide or native nucleic acid sequence of a gene coding for a polypeptide described herein. The wild-type sequence of a protein or gene is the most common sequence of the polypeptide or gene for a species for that protein or gene.

The term “chimeric receptors,” as used herein, refers to a transmembrane receptor that is engineered to have at least a portion of at least one domain (e.g., ECD, ICD, TMD, or C-terminal region) that is derived from sequences of one or more different transmembrane proteins or receptors.

The term “operatively linked” refers to nucleic acid or amino acid sequences that are placed into a functional relationship with another nucleic acid or amino acid sequence, respectively. Generally, “operatively linked” means that nucleic acid sequences or amino acid sequences being linked are contiguous, and, in the case of a secretory leader, contiguous and in reading phase.

As used herein, the term “extracellular domain” (ECD) refers to the domain of a receptor (e.g., G-CSFR) that, when expressed on the surface of a cell, is external to the plasma membrane. In certain embodiments the ECD of G-CSFR comprises at least a portion of SEQ ID 2 or 7.

As used herein, the term “intracellular domain” (ICD) refers to the domain of a receptor that is located within the cell when the receptor is expressed on a cell surface.

As used herein, the term “transmembrane domain” (TMD or TM) refers to the domain or region of a cell surface receptor that is located within the plasma membrane when the receptor is expressed on a cell surface.

The term “cytokine” refers to small proteins (about 5-20 kDa) that bind to cytokine receptors and can induce cell signaling upon binding to and activation of a cytokine receptor expressed on a cell. Examples of cytokines include, but are not limited to, interleukins, lymphokines, colony stimulating factors and chemokines.

The term “cytokine receptor” refers to receptors that bind to cytokines, including type 1 and type 2 cytokine receptors. Cytokine receptors include, but are not limited to, IL-2R (Interleukin-2 receptor), IL-7R (Interleukin-7 receptor), IL-12R (Interleukin-12 Receptor), and IL-21R (Interleukin-21 Receptor).

The term “G-CSFR” refers to Granulocyte Colony-Stimulating Factor Receptor. G-CSFR can also be referred to as: GCSFR, G-CSF Receptor, Colony Stimulating Factor 3 Receptor, CSF3R, CD114 Antigen, or SCN7. Human G-CSFR is encoded by the gene having an Ensembl identification number of: ENSG00000119535. Human G-CSFR is encoded by the cDNA sequence corresponding to GeneBank Accession number NM_156039.3.

The term “G-CSF” refers to Granulocyte Colony Stimulating Factor. G-CSF can also be called Colony Stimulating Factor 3 and CSF3. Human G-CSF is encoded by the gene having an Ensembl identification number of: ENSG00000108342. Human G-CSF is encoded by the cDNA sequence corresponding to GeneBank Accession number KP271008.1.

The term “at least a portion of” refers to greater than 50%, greater than 75%, greater than 80%, greater than 90%, greater than 95%, greater than 99% of the length of contiguous nucleotides or amino acids of a SEQ ID NO described herein. The at least a portion of a domain or binding site (e.g., ECD, ICD, transmembrane, C-terminal region or signaling molecule binding site) described herein can be greater than 50%, greater than 75%, greater than 80%, greater than 90%, greater than 95%, greater than 99% identical to a SEQ ID NO described herein.

The term “signaling molecule binding site” refers to a nucleotide or amino acid sequence of a cytokine receptor intracellular domain that is required for or increases the cytokine receptor binding to a downstream signaling molecule.

The term “Box 1” or “Box 2” region refers to a region on an ICD that serves as a binding site for the tyrosine kinases Jak1, Jak2, Jak3 or Tyk2. The Box 1 region may comprise a sequence of amino acids that is greater than 50% identical to a Box 1 sequence listed in Table 2.

The term “C terminal region” refers to the carboxy-terminal region of the cytokine receptor that includes at least one signaling molecule binding site of the chimeric receptor.

The terms “orthogonal,” or “orthogonal cytokine-receptor pair” refers to genetically engineered pairs of proteins that are modified by amino acid changes to (a) lack binding to the native cytokine or cognate receptor; and (b) to specifically bind to the counterpart engineered (orthogonal) ligand or receptor.

The term “orthogonal receptor,” as used herein, refers to the genetically engineered receptor of an orthogonal cytokine-receptor pair.

The term “orthogonal cytokine,” or “orthogonal G-CSF,” as used herein, refers to the genetically engineered cytokine of an orthogonal cytokine-receptor pair.

As used herein, “do not bind”, “does not bind” or “incapable of binding” refers to no detectable binding, or an insignificant binding, i.e., having a binding affinity much lower than that of the natural ligand.

A cytokine that can “selectively activate a chimeric receptor” refers to a cytokine that preferentially binds to and activates a chimeric receptor compared to the native (wild-type) cytokine receptor. In certain aspects, the cytokine selectively activates a chimeric receptor that is the orthogonal counterpart of the orthogonal cytokine-receptor pair. In certain aspects, the cytokine is a wild-type cytokine and it selectively activates a chimeric receptor that is expressed on cells, whereas the native, wild-type receptor to the cytokine is not expressed in the cells.

The term “immune cell” refers to any cell that is known to function to support the immune system of an organism (including innate and adaptive immune responses), and includes, but is not limited to, Lymphocytes (e.g., B cells, plasma cells and T cells), Natural Killer Cells (NK cells), Macrophages, Monocytes, Dendritic cells, Neutrophils, and Granulocytes. Immune cells include stem cells, immature immune cells and differentiated cells. Immune cells also include any sub-population of cells, however rare or abundant in an organism. In certain embodiments, an immune cell is identified as such by harboring known markers (e.g., cell surface markers) of immune cell types and sub-populations.

The term, “enhanced activity,” as used herein, refers to increased activity of a variant receptor expressed on a cell upon stimulation with a variant cytokine, wherein the activity is an activity observed for a native receptor upon stimulation with a native cytokine.

The term “T cells” refers to mammalian immune effector cells that may be characterized by expression of CD3 and/or T cell antigen receptor, which cells may be engineered to express an orthologous cytokine receptor. In some embodiments, the T cells are selected from naïve CD8⁺ T cells, cytotoxic CD8⁺ T cells, naïve CD4⁺ T cells, helper T cells, e. g., T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, effector memory T cells, NKT cells, and γδT cells.

Abbreviations used in this application include the following: ECD (extracellular domain), ICD (intracellular domain), TMD or TM (transmembrane domain), a G-CSFR (Granulocyte-Colony Stimulating Factor Receptor), a G-CSF (Granulocyte-Colony Stimulating Factor), IL-2R (Interleukin-2 receptor), IL-12R (Interleukin 12 Receptor), IL-21R (Interleukin-21 Receptor) and IL-7R (interleukin-7 receptor), and NK cell (Natural Killer Cell). IL-2Rγ can also be referred to herein as: IL-2RG, IL-2Rgc, γc, or IL-2Rγc.

“JAK” can also be referred to as Janus Kinase. JAK is a family of intracellular, nonreceptor tyrosine kinases that transduce cytokine-mediated signals via the Jak-STAT pathway and includes JAK1, JAK2, JAK3 and TYK2. Human JAK1 is encoded by the gene having an Ensembl identification number of: ENSG00000162434. Human JAK1 is encoded by the cDNA sequence corresponding to GeneBank Accession number NM_002227. Human JAK2 is encoded by the gene having an Ensembl identification number of: ENSG00000096968. Human JAK2 is encoded by the cDNA sequence corresponding to GeneBank Accession number_NM_001322194. Human JAK3 is encoded by the gene having an Ensembl identification number of: ENSG00000105639. Human JAK3 is encoded by the cDNA sequence corresponding to GeneBank Accession number NM_000215. Human TYK2 is encoded by the gene having an Ensembl identification number of: ENSG00000105397. Human TYK2 is encoded by the cDNA sequence corresponding to GeneBank Accession number NM_001385197.

STAT can also be referred to as Signal Transducer and Activator of Transcription. STAT is a family of 7 STAT proteins: STAT1, STAT2, STAT3, STAT4, STAT5A, STAT5B and STAT6. Human_STAT1 is encoded by the gene having an Ensembl identification number of: ENSG00000115415. Human STAT1 is encoded by the cDNA sequence corresponding to GeneBank Accession number NM_007315. Human_STAT2 is encoded by the gene having an Ensembl identification number of: ENSG00000170581. Human STAT2 is encoded by the cDNA sequence corresponding to GeneBank Accession number NM_005419. Human_STAT3 is encoded by the gene having an Ensembl identification number of: ENSG00000168610. Human STAT3 is encoded by the cDNA sequence corresponding to GeneBank Accession number NM_139276. Human STAT4 is encoded by the gene having an Ensembl identification number of: ENSG00000138378. Human STAT4 is encoded by the cDNA sequence corresponding to GeneBank Accession number NM_003151. Human STAT5A is encoded by the gene having an Ensembl identification number of: ENSG00000126561. Human STAT5A is encoded by the cDNA sequence corresponding to GeneBank Accession number NM_003152. Human_STAT5B is encoded by the gene having an Ensembl identification number of: ENSG00000173757. Human STAT5B is encoded by the cDNA sequence corresponding to GeneBank Accession number NM_012448. Human_STAT6 is encoded by the gene having an Ensembl identification number of: ENSG00000166888. Human STAT6 is encoded by the cDNA sequence corresponding to GeneBank Accession number NM_003153.

SHC can also be referred to as Src Homology 2 Domain Containing Transforming Protein. Shc is a family of three isoforms and includes p66Shc, p52Shc and p46Shc, SHC1, SHC2 and SHC3. Human SHC1 is encoded by the gene having an Ensembl identification number of: ENSG00000160691. Human SHC1 is encoded by the cDNA sequence corresponding to GeneBank Accession number NM_183001. Human_SHC2 is encoded by the gene having an Ensembl identification number of: ENSG00000129946. Human SHC2 is encoded by the cDNA sequence corresponding to GeneBank Accession number NM_012435. Human_SHC3 is encoded by the gene having an Ensembl identification number of: ENSG00000148082. Human SHC3 is encoded by the cDNA sequence corresponding to GeneBank Accession number NM_016848.

SHP-2 can also be referred to as Protein Tyrosine Phosphatase Non-Receptor Type 11 (PTPN11) and Protein-Tyrosine Phosphatase 1D (PTP-1D). Human SHP-2 is encoded by the gene having an Ensembl identification number of: ENSG00000179295. Human SHP-2 is encoded by the cDNA sequence corresponding to GeneBank Accession number NM_001330437.

PI3K can also be referred to as Phosphatidylinositol-4,5-Bisphosphate 3-Kinase. The catalytic subunit of PI3K can be referred to as PIK3CA. Human PIK3CA is encoded by the gene having an Ensembl identification number of: ENSG00000121879. Human PIK3CA is encoded by the cDNA sequence corresponding to GeneBank Accession number NM_006218.

It must be noted that, as used in the specification and the appended claims, the singular forms “a,” “an” and “the” include plural referents unless the context clearly dictates otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### Chimeric Cytokine Receptor Designs

In certain embodiments, described herein are chimeric cytokine receptors comprising an extracellular domain (ECD) of a G-CSFR (Granulocyte-Colony Stimulating Factor Receptor) operatively linked to a second domain; the second domain comprising at least a portion of an intracellular domain (ICD) of a multi-subunit cytokine receptor, e.g., IL-2R. In certain aspects, the chimeric cytokine receptor comprises a portion of an ICD from Table 1A and Table 1B. In certain aspects, the chimeric cytokine receptor comprises a transmembrane domain selected from Table 1A and Table 1B. In certain aspects, the chimeric cytokine receptor ICD comprises Box1 and Box 2 regions from Table 1A, Table 1B and Table 2. In certain aspects, the chimeric cytokine receptor comprises at least one signaling molecule binding site from Table 1A, Table 1B and Table 2.

In certain aspects, the chimeric receptors described herein comprise ECD domains that share at least 80%, at least 85%, at least 90%, at least 95%, at least 96%, at least 97%, at least 98%, or at least 99% amino acid or nucleic acid sequence identity to an ECD SEQ ID NO. described herein. In certain aspects, the chimeric receptor comprises the ECD of G-CSFR having an amino acid sequence of SEQ ID NO. 5 or a nucleic acid sequence of SEQ ID NO. 6 or 7. In certain aspects, the chimeric receptor comprises the ECD of G-CSFR, wherein the ECD comprises at least one amino acid substitution. In certain aspects, the ECD of G-CSFR comprises at least one amino acid substitution selected from the group consisting of R41E, R141E, and R167D.

In certain aspects, the chimeric receptors described herein comprise transmembrane domains (TMD) that share at least 80%, at least 85%, at least 90%, at least 95%, at least 96%, at least 97%, at least 98%, or at least 99% amino acid or nucleic acid sequence identity to a TMD SEQ ID NO. described herein. In certain aspects, the chimeric receptor comprises the TMD of gp130 having an amino acid sequence of SEQ ID NO. 9 or a nucleic acid sequence of SEQ ID NO. 13. In certain aspects, the chimeric receptor comprises the TMD of G-CSFR having an amino acid sequence of SEQ ID NO. 8 or a nucleic acid sequence of SEQ ID NO. 12.

In certain aspects, the chimeric receptors described herein comprise at least a portion of an ICD of a cytokine receptor that shares at least 80%, at least 85%, at least 90%, at least 95%, at least 96%, at least 97%, at least 98%, or at least 99% amino acid or nucleic acid sequence identity to an ICD SEQ ID NO. described herein. In certain aspects, the chimeric receptor comprises at least a portion of an ICD of IL-2Rβ having an amino acid sequence of SEQ ID NO. 16, 19, 21, 29, 31, 33, 35, 37, or 39. In certain aspects, the chimeric receptor comprises at least a portion of an ICD of IL-2Rβ having a nucleic acid sequence of SEQ ID NO. 44, 47, 49, 57, 59, 61, 63, 65, or 67. In certain aspects, the chimeric receptor comprises at least a portion of an ICD of IL-7Rα having an amino acid sequence of SEQ ID NO. 41 or a nucleic acid sequence of SEQ ID NO. 69. In certain aspects, the chimeric receptor comprises at least a portion of an ICD of TL-7R having an amino acid sequence of SEQ ID NO. 43 or a nucleic acid sequence of SEQ ID NO. 71. In certain aspects, the chimeric receptor comprises at least a portion of an ICD of IL-21R having an amino acid sequence of SEQ ID NO. 35 or 45. In certain aspects, the chimeric receptor comprises at least a portion of an ICD of IL-21R having a nucleic acid sequence of SEQ ID NO. 25 or 27. In certain aspects, the chimeric receptor comprises at least a portion of an ICD of IL-12Rβ₂ having an amino acid sequence of SEQ ID NO. 23, 32, or 36. In certain aspects, the chimeric receptor comprises at least a portion of an ICD of IL-12Rβ₂ having a nucleic acid sequence of SEQ ID NO. 51, 60 or 64. In certain aspects, the chimeric receptor comprises at least a portion of an ICD of G-CSFR having an amino acid sequence of SEQ ID NO. 20, 22, 24, 26, 28, 30, 34, 40 or 42. In certain aspects, the chimeric receptor comprises at least a portion of an ICD of G-CSFR having a nucleic acid sequence of SEQ ID NO. 48, 50, 52, 54, 56, 58, 62, 68 or 70. In certain aspects, the chimeric receptor comprises at least a portion of an ICD of gp130 having an amino acid sequence of SEQ ID NO. 18 or 38. In certain aspects, the chimeric receptor comprises at least a portion of an ICD of gp130 having a nucleic acid sequence of SEQ ID NO. 46 or 66. In certain aspects, the chimeric receptor comprises at least a portion of an ICD of IL-2Rγ (i.e., IL-2RG, IL-2Rgc, γc or IL-2Rγc) having an amino acid sequence of SEQ ID NO. 17. In certain aspects, the chimeric receptor comprises at least a portion of an ICD of IL-2Rγ (i.e., IL-2RG, IL-2Rgc, γc or IL-2Rγc) having a nucleic acid sequence of SEQ ID NO. 45.

In certain aspects, the at least a portion of the ICDs described herein comprise at least one signaling molecule binding site. In certain aspects, the at least one signaling molecule binding site is a STAT3 binding site of G-CSFR; a STAT3 binding site of gp130; a SHP-2 binding site of gp130; a Shc binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT5 binding site of IL-12Rβ₂; a STAT4 binding site of IL-12Rβ₂; a STAT3 binding site of IL-12Rβ₂; a STAT5 binding site of IL-21R; a STAT3 binding site of IL-21R; and a STAT1 binding site of IL-21R. In certain aspects, the at least one signaling molecule binding site comprises a sequence that further comprises an amino acid listed in Table 2.

In certain aspects, the at least a portion of the ICDs described herein comprise the Box 1 and Box regions of gp130 or G-CSFR. In certain aspects the Box 1 region comprises a sequence of amino acids listed in Table 2. In certain aspects the Box 1 region comprises an amino acid sequence that is greater than 50% identical to a Box 1 sequence listed in Table 2.

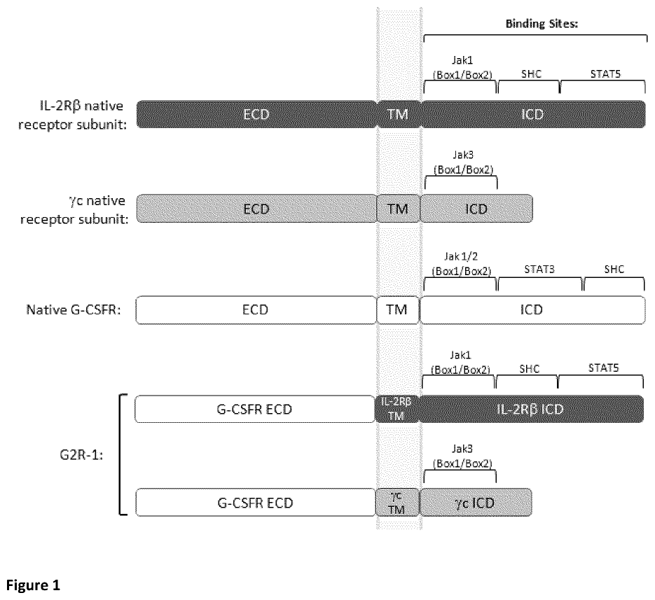

In certain aspects, the chimeric receptors described herein comprise a G-CSFR ECD domain, a transmembrane domains (TMD), and at least one portion of one ICD arranged in N-terminal to C-terminal order, as shown in a chimeric receptor design of FIGS. 1, 4 and 5.

In certain aspects, the chimeric receptors described herein comprise amino acid sequences in N-terminal to C-terminal order of the sequences disclosed in each of Tables 3-6. In certain aspects, the sequences of the chimeric receptors described herein comprise nucleic acid sequences in 5′ to 3′ order of the sequences disclosed in each of Tables 3-6. In certain aspects, the chimeric cytokine receptor shares at least 50%, at least 60%, at least 70%, at least 80%, at least 85%, at least 90%, at least 95%, at least 96%, at least 97%, at least 98%, or at least 99% amino acid identity to the amino acid sequences in N-terminal to C-terminal order of the amino acid sequences disclosed in each of Tables 3-6. In certain aspects, the chimeric cytokine receptor shares at least 50%, at least 60%, at least 70%, at least 80%, at least 85%, at least 90%, at least 95%, at least 96%, at least 97%, at least 98%, or at least 99% nucleic acid identity to the nucleic acid sequences in 5′ to 3′ order of the nucleic acid sequences disclosed in each of Tables 3-6.

TABLE 1A
Chimeric Cytokine Receptors

| | | | | | STAT | Other |
|---|---|---|---|---|---|---|
| Name | ECD | TMD | Box1/2 | ICD | site(s) | site(s) |
| G2R-1 | G-CSFR | IL-2Rβ + γc | IL-2Rβ + γc | IL-2Rβ + γc | 5, 5 | Shc |
| G2R-2 | G-CSFR | gp130 | gp130 | IL-2Rβ | 5, 5 | Shc |
| G2R-3 | G-CSFR | G-CSFR | G-CSFR | IL-2Rβ | 5, 5 | Shc |
| G21R-1 | G-CSFR | G-CSFR | G-CSFR | IL-21R | 3 | |
| G21R-2 | G-CSFR | G-CSFR | G-CSFR | IL-21R | 3 | unknown |
| G12R-1 | G-CSFR | G-CSFR | G-CSFR | IL-2Rβ₂ | 4 | |

TABLE 1B
Chimeric Cytokine Receptors.

| | | | | | STAT | Other |
|---|---|---|---|---|---|---|
| Name | ECD | TMD | Box1/2 | ICD | site(s) | site(s) |
| G21/2R-1 | G-CSFR | G-CSFR | G-CSFR | G-CSFR, IL-2Rβ | 3, 5, 5 | Shc |
| G12/2R-1 | G-CSFR | G-CSFR | G-CSFR | STAT4 site from IL- | 4, 5 | Shc |
| | | | | 12Rβ₂ replaces | | |
| | | | | STAT5 site from IL- | | |
| | | | | 2Rβ | | |
| G21/12/2R-1 | G-CSFR | G-CSFR | G-CSFR | Contains STAT3 site | 3, 4, 5 | Shc |
| | | | | from G-CSFR ICD; | | |
| | | | | STAT4 site from IL- | | |
| | | | | 12Rβ₂ replaces | | |
| | | | | STAT5 site from IL- | | |
| | | | | 2Rβ | | |
| G27/2R-1 | G-CSFR | gp130 | gp130 | gp130 + IL-2Rβ | 3, 5, 5 | SHP-2, |
| | | | | | | Shc |
| G7R-1 | G-CSFR | G-CSFR | G-CSFR | IL-7Rα | 5 | PI3K |

TABLE 2

| | | SEQ | | | | | | | |
|---|---|---|---|---|---|---|---|---|---|
| Native | | ID | | | | | | | |
| ICD | Box 1 (source) | NO: | Shc | SHP-2 | STAT5 | STAT4 | STAT3 | STAT1 | PI3K |
| G- | LWPSVPDPA | 74 | n/a | n/a | n/a | n/a | Y704 | n/a | n/a |
| CSFR | (Q99062) | | | | | | | | |
| gp130 | IWPNVPDPS | 75 | n/a | Y759 | n/a | n/a | Y767 | n/a | n/a |
| | (P40189) | | | | | | | | |
| IL- | LKCNTPDPS | 76 | Y338 | n/a | Y392, | n/a | Y392, | Y392, | n/a |
| 2Rβ | (P14784) | | | | Y510 | | Y510 | Y510 | |
| IL- | KIWAVPSPE | 77 | n/a | n/a | n/a | n/a | Y510 | Y510 | n/a |
| 21R | (Q9HBE5) | | | | | | | | |
| IL- | CSREIPDPA | 78 | n/a | n/a | Y800 | Y800 | Y800 | n/a | n/a |
| 12Rβ₂ | (Q99665) | | | | | | | | |
| IL- | VWPSLPDHK | 79 | n/a | n/a | Y449 | n/a | n/a | n/a | Y449 |
| 7Rα | (P16871) | | | | | | | | |

Ligands for Chimeric Cytokine Receptors

Described herein are ligands that specifically bind the chimeric receptors described herein. In certain aspects, the ligand is a wild-type ligand. In certain aspects, the ligand is an orthogonal cytokine (i.e., a variant cytokine) that binds with higher affinity to a chimeric receptor compared to binding to a wild type receptor. In certain aspects, the ligand is a wild-type G-CSF. In certain aspects, the ligand is a G-CSF with one or more amino acid substitutions, e.g., one or more amino acid substitutions selected from the group consisting of E46R, L108K, and D112R.

Upon binding of the orthogonal cytokine to the orthogonal chimeric receptor on the cell surface, the chimeric receptor activates signaling that is transduced through native cellular elements to provide for a biological activity that mimics that native response, but which is specific to a cell engineered to express the orthogonal chimeric receptor. The orthogonal chimeric receptor does not bind to the endogenous counterpart cytokine, including the native counterpart of the orthogonal cytokine, while the orthogonal cytokine does not bind to any endogenous receptors, including the native counterpart of the chimeric receptor. In certain embodiments, the orthogonal cytokine binds the native receptor with significantly reduced affinity compared to binding of the native cytokine to the native cytokine receptor. In certain embodiments, the affinity of the orthogonal cytokine for the native receptor is less than 10×, less than 100×, less than 1,000× or less than 10,000× of the affinity of the native cytokine to the native cytokine receptor. In certain embodiments, the orthogonal cytokine binds the native receptor with a K_{D} of greater than 1×10⁻⁴ M, 1×10⁻⁵ M, greater than 1×10⁻⁶ M; greater than 1×10⁻⁷ M, greater than 1×10⁻⁸ M, or greater than 1×10⁻⁹ M. In certain embodiments, the orthogonal cytokine receptor binds the native cytokine with significantly reduced affinity compared to the binding of the native cytokine receptor to the native cytokine. In certain embodiments, the orthogonal cytokine receptor binds the native cytokine less than 10×, less than 100×, less than 1,000× or less than 10,000× the native cytokine to the native cytokine receptor. In certain embodiments, the orthogonal cytokine receptor binds the native cytokine with a K_{D} of greater than 1×10⁻⁴ M, 1×10⁻⁵ M, greater than 1×10⁻⁶ M; greater than 1×10⁻⁷ M, greater than 1×10⁻⁸ M, or greater than 1×10⁻⁹ M. In certain embodiments, the affinity of the orthogonal cytokine for the orthogonal chimeric receptor is comparable to the affinity of the native cytokine for the native receptor, e.g., having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor.

The affinity can be determined by any number of assays well known to one of skill in the art. For example, affinity can be determined with competitive binding experiments that measure the binding of a receptor using a single concentration of labeled ligand in the presence of various concentrations of unlabeled ligand. Typically, the concentration of unlabeled ligand varies over at least six orders of magnitude. Through competitive binding experiments, IC₅₀ can be determined. As used herein, “IC₅₀” refers to the concentration of the unlabeled ligand that is required for 50% inhibition of the association between receptor and the labeled ligand. IC₅₀ is an indicator of the ligand-receptor binding affinity. Low IC₅₀ represents high affinity, while high IC₅₀ represents low affinity.

Binding of an orthogonal ligand to the chimeric cytokine receptor expressed on the surface of a cell, may or may not affect the function of the cytokine receptor (as compared to native cytokine receptor activity); native activity is not necessary or desired in all cases. In certain embodiments, the binding of an orthogonal ligand to the chimeric cytokine receptor will induce one or more aspects of native cytokine signaling. In certain embodiments, the binding of an orthogonal cytokine to the chimeric cytokine receptor expressed on the surface of a cell causes a cellular response selected from the group consisting of proliferation, viability and enhanced activity.

### Nucleic Acids Encoding Chimeric Cytokine Receptors

Included in this disclosure are nucleic acids encoding any one of the chimeric cytokine receptors described herein. Described herein are expression vectors, or kits of expression vectors, which comprise one or more nucleic acid sequence(s) encoding a one or more chimeric cytokine receptor(s) described herein.

The nucleic acid encoding a chimeric cytokine receptor is inserted into a replicable vector for expression. Such a vector may be used to introduce the nucleic acid sequence(s) into a host cell so that it expresses a chimeric cytokine receptor described herein. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like. The vector can be, e.g., a retroviral vector, adenoviral vector, lentiviral vector, a transposon-based vector, or a synthetic mRNA. The vector may be capable of transfecting or transducing a T cell, an NK cell or any other immune or non-immune cells.

A chimeric cytokine receptor may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence can be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders. In certain aspects, the signal sequence is a nucleic acid sequence of SEQ ID NO. 6 or an amino acid sequence of SEQ ID NO. 1.

### Expression Vectors Encoding Chimeric Cytokine Receptors

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors contain a promoter that is recognized by the host organism and is operably linked to an orthologous protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are known from mammalian genes (globin, elastase, albumin, fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

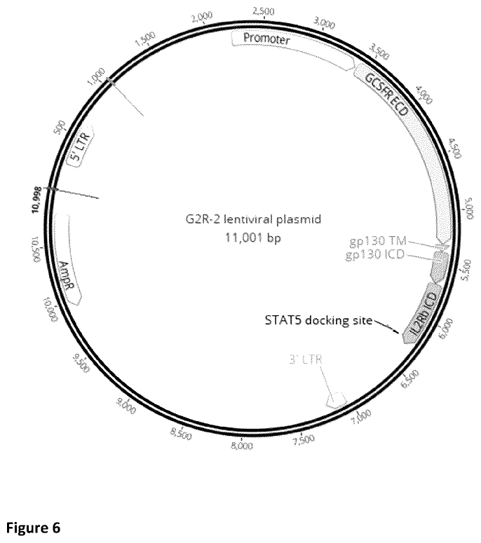

In certain aspects, disclosed herein are lentiviral vectors encoding the chimeric receptors disclosed herein. In certain aspects the lentiviral vector comprises the HIV-1 5′ LTR and a 3′ LTR. In certain aspects, the lentiviral vector comprises an EF1a promoter. In certain aspects, the lentiviral vector comprises an SV40 poly a terminator sequence. In certain aspects the lentiviral vector is the vector of FIG. 6. In certain aspects, the vector is psPAX2, Addgene® 12260, pCMV-VSV-G, or Addgene® 8454.

Described herein are nucleic acid and polypeptide sequences. In some embodiments, also described are nucleic acid and polypeptide sequence with high sequence identity, e.g., 95, 96, 97, 98, 99% or more sequence identity to sequences described herein. The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Cells Expressing Chimeric Receptors

Also described herein are cells expressing the chimeric receptors. Host cells, including engineered immune cells, can be transfected or transduced with the above-described expression vectors for cytokine or chimeric cytokine receptor expression.

The present invention provides a cell which comprises one or more chimeric cytokine receptors. The cell may comprise a nucleic acid or a vector encoding the chimeric cytokine receptors described herein. This disclosure also provides methods of producing cells expressing chimeric cytokine receptor. In certain aspects, the cells are produced by introducing into a cell the nucleic acid or expression vector described herein. The cells can be introduced to the nucleic acid or expression vector by any process including, but not limited to, transfection, transduction of a viral vector, transposition or gene editing. Any gene editing technique known in the art may be used including, but not limited to, techniques comprising clustered regularly interspaced short palindromic repeats (CRISPR-Cas) systems, zinc finger nucleases, transcription activator-like effector-based nucleases and meganucleases.

The host cell can be any cell in the body. In certain embodiments, the cell is an immune cell. In some embodiments, the cell is a T cell, including, but not limited to, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, effector memory T cells, NKT cells, γδT cells; etc. In certain embodiments, the cell is a B cell, including, but not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc. In certain embodiments, the cell is an innate lymphoid cell, including, but not limited to, NK cells, etc. In certain embodiments, the cell is a myeloid cell, including, but not limited to, macrophages, dendritic cells, myeloid-derived suppressor cells, etc.

In certain embodiments, the cell is a stem cell, including, but not limited to, hematopoietic stem cells, mesenchymal stem cells, neural stem cells, etc.

In some embodiments, the cell is genetically modified in an ex vivo procedure, prior to transfer into a subject. The cell can be provided in a unit dose for therapy, and can be allogeneic, autologous, etc. with respect to an intended recipient.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarized below.

In certain aspects, the cells expressing chimeric cytokine receptors described herein are helper T helper cells (Th cells). Th cells assist other white blood cells in immunologic processes, including maturation of B cells into plasma cells and memory B cells, and activation of cytotoxic T cells and macrophages. Th cells usually express CD4 on their surface. Th cells become activated when they are presented with peptide antigens by MHC class II molecules on the surface of antigen presenting cells (APCs). These cells can differentiate into one of several subtypes, including Th1, Th2, Th17, Th9, or Tfh, which secrete different cytokines to facilitate different types of immune responses.

In certain aspects, the cells expressing chimeric cytokine receptors described herein are cytolytic T cells (TC cells, or CTLs). CTLs destroy virally infected cells and tumor cells, and are also implicated in transplant rejection. CTLs usually express CD8 on their surface. These cells recognize their targets by binding to antigen associated with MHC class I, which is present on the surface of all healthy nucleated cells.

In certain aspects, the cells expressing chimeric cytokine receptors described herein are memory T cells. Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise at least three subtypes: central memory T cells (TCM cells) and two types of effector memory T cells (TEM cells and TEMRA cells). Memory cells may be either CD4+ or CD8+. Memory T cells typically express the cell surface protein CD45RO.

In certain aspects, the cells expressing chimeric cytokine receptors described herein are regulatory T cells (Treg cells). Treg cells, formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus. Two major classes of CD4+ Treg cells have been described: naturally occurring Treg cells and adaptive (or induced) Treg cells. Naturally occurring Treg cells (also known as CD4+CD25+FoxP3+ Treg cells) arise in the thymus and have been linked to interactions between developing T cells with both myeloid (CD1 1c+) and plasmacytoid (CD123+) dendritic cells that have been activated with TSLP. Treg cells can be distinguished from other T cells by the presence of an intracellular molecule called FoxP3.

In certain aspects, the cells expressing chimeric cytokine receptors described herein are tumor-infiltrating lymphocytes (TILs) or tumor-associated lymphocytes (TALs). In certain aspects, the TILs/TALs comprise CD4+, T cells, CD8+ T cells, Natural Killer (NK) cells, and combinations thereof.

In certain embodiments, the T cells described herein are chimeric antigen receptor T cells (CAR-T cells) that have been genetically engineered to produce an artificial T-cell receptor for use in immunotherapy. In certain aspects, the CAR-T cells are derived from T cells in a patient's own blood (i.e., autologous). In certain aspects, the CAR-T cells are derived from the T cells of a donor (i.e., allogeneic).

In certain embodiments, the T cells described herein are engineered T Cell Receptor (eTCR-T cells) that have been genetically engineered to produce a particular T Cell Receptor for use in immunotherapy. In certain aspects, the eTCR-T cells are derived from T cells in a patient's own blood (i.e., autologous). In certain aspects, the eTCR-T cells are derived from the T cells of a donor (i.e., allogeneic).

In certain aspects, the cells expressing chimeric cytokine receptors described herein are a Natural Killer cell (NK cell). NK cells form part of the innate immune system. NK cells provide rapid responses to innate signals from virally infected cells in an MHC independent manner. NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

In certain aspects, the cells expressing chimeric cytokine receptors described herein are B cells. B cells include, but are not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc.

In certain aspects, the cells expressing chimeric cytokine receptors described herein are myeloid cells, including, but not limited to, macrophages, dendritic cells, myeloid-derved suppressor cells, etc.

The cells expressing a variant receptor or variant cytokine described herein may be of any cell type. In certain aspects, the cells expressing a chimeric receptor or cytokine described herein is a cell of the hematopoietic system. Immune cells (e.g., T cells or NK cells) may be derived ex vivo either from a patient's own peripheral blood (1st party), or in the setting of a hematopoietic stem cell transplant from donor peripheral blood (2nd party), or peripheral blood from an unconnected donor (3rd party). Alternatively, immune cells may be derived from ex vivo differentiation of inducible progenitor cells or embryonic progenitor cells to immune cells. Alternatively, an immortalized immune cell line which retains its effector function (e.g., a T-cell or NK-cell line that retains its lytic function; a plasma cell line that retains its antibody producing function; or a dendritic cell line or macrophage that retains its phagocytic and antigen presentation function) and could act as a therapeutic may be used. In all these embodiments, cells expressing chimeric cytokine receptor are generated by introducing DNA or RNA coding for each chimeric cytokine receptor(s) by one of many means including transduction with a viral vector, or transfection with DNA or RNA.

The cells can be immune cells derived from a subject and engineered ex vivo to express a chimeric cytokine receptor and/or cytokine. The immune cells may be from a peripheral blood mononuclear cell (PBMC) sample. Immune cells may be activated and/or expanded prior to being transduced with nucleic acid encoding the molecules providing the chimeric cytokine receptor according to the first aspect of the invention, for example by treatment with an anti-CD3 monoclonal antibody. The immune cells of the invention may be made by: (i) isolation of an immune cell-containing sample from a subject or other sources listed above; and (ii) transduction or transfection of the immune cells with one or more nucleic acid sequence(s) encoding a chimeric cytokine receptor(s).

Cells can be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

The immune cells may then be purified, for example, by selecting on the basis of expression of an antigen-binding domain of an antibody. In certain embodiments, the cells are selected by a expression of a selectable marker (e.g., a protein, a fluorescent marker, or an epitope tag) or by any method known in the art for selection, isolation and/or purification of the cells.

### Kits

This disclosure also describes kits for producing a cell expressing at least one of the chimeric cytokine receptors described herein. In certain embodiments, the kit comprises at least one expression vector encoding at least one chimeric cytokine receptor and instructions for use. In certain aspects, the kits further comprise at least one cytokine in a pharmaceutical formulation or an expression vector encoding a cytokine that binds to at least one of the chimeric cytokine receptors described herein. In certain embodiments, the kits comprise a cell comprising an expression vector encoding a chimeric receptor described herein.

In certain embodiments, the kits comprise a cell comprising an expression vector encoding a variant receptor described herein. In certain embodiments, the kits comprise a cell comprising an expression vector encoding a Chimeric Antigen Receptor (CAR)/engineered T cell receptor (eTCR) or the like (e.g., engineered non-native TCR receptors). In certain embodiments the kits comprise an expression vector encoding a Chimeric Antigen Receptor (CAR)/engineered T cell receptor (eTCR) or the like. In certain embodiments the kits comprise an expression vector encoding a variant receptor described herein and a Chimeric Antigen Receptor (CAR)/engineered T cell receptor (eTCR) or the like.

In certain aspects, the kits described herein further comprise an orthogonal cytokine. In certain aspects, the kits further comprise an at least one cytokine in a pharmaceutical formulation. In certain embodiments, the kit further comprises at least one additional cytokine. In certain embodiments, the components are provided in a dosage form, in liquid or solid form in any convenient packaging.

Additional reagents may be provided for the growth, selection and preparation of the cells provided or cells produced as described herein. For example, the kit can include components for cell culture, growth factors, differentiation agents, reagents for transfection or transduction, etc.

In certain embodiments, in additional to the above components, the kits may also include instructions for use. Instructions can be provided in any convenient form. For example, the instructions may be provided as printed information, in the packaging of the kit, in a package insert, etc. The instructions can also be provided as a computer readable medium on which the information has been recorded. In addition, the instructions may be provided on a website address which can be used to access the information.

### Methods of Selective Activation of a Chimeric Receptor

This disclosure provides methods for selective activation of a chimeric cytokine receptor expressed on the surface of a cell, comprising contacting the chimeric cytokine receptor described herein with a cytokine that selectively activates the chimeric receptor. In certain aspects, the cytokine that selectively activates the chimeric receptor is a G-CSF. The G-CSF can be a wild-type G-CSF or a G-CSF comprising one or more mutations that confers preferential binding and activation of the G-CSF to the chimeric receptor compared to the native (wild-type) cytokine receptor.

In certain aspects, the selective activation of the chimeric receptor by binding of the cytokine to the chimeric receptor leads to homodimerization of the receptor, heterodimerization of the receptor, or combinations thereof. In certain aspects, activation of the chimeric cytokine receptor leads to activation of downstream signaling molecules. In certain aspects, the activation of the downstream signaling molecules includes activation of cellular signaling pathways that stimulate cell cycle progression, proliferation, viability and/or functional activity of the cell. In certain aspects, the signaling pathways or molecules that are activated are, but not limited to, Jak1, Jak2, Jak3, STAT1, STAT2, STAT3, Shc, ERK1/2 and Akt. In certain aspects, activation of the chimeric cytokine receptor leads to increased proliferation of the cell after administration of a cytokine that binds the receptor. In certain aspects, the extent of proliferation is between 1-1,000 fold, 1-100 fold, 1-50 fold, 1-10 fold, 1-5 fold, 1-2 fold, 1-1.5, or 0.1-10 fold the proliferation observed when the cells are stimulated with IL-2.

### Methods Using Stem Cells Expressing Variant Cytokine Receptors

The present invention provides a method for treating and/or preventing a condition or disease which comprises the step of administering stem cells expressing a chimeric cytokine receptor and/or orhtogonal cytokine described herein. In certain embodiments, stem cells expressing the variant cytokine receptors and/or variant cytokines described herein are used for regenerative medicine, cell/tissue/organ transplantation, tissue reconstruction, or tissue repair.

### Methods of Adoptive Cell Transfer

The present invention provides a method for treating and/or preventing a disease which comprises the step of administering cells expressing a chimeric cytokine receptor described herein (for example, in a pharmaceutical composition as described below) to a subject.

A method for treating and/or preventing a disease relates to the therapeutic use of the cells described herein, e.g., T cells, NK cells, or any other immune or non-immune cells expressing the chimeric cytokine receptor. The cells can be administered to a subject having an existing disease or condition in order to lessen, reduce or improve at least one symptom associated with the disease and/or to slow down, reduce or block the progression of the disease. The method for preventing a disease relates to the prophylactic use of the cells of the present invention. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

In some embodiments, the subject compositions, methods and kits are used to enhance an immune response. In some embodiments the immune response is directed towards a condition where it is desirable to deplete or regulate target cells, e.g., cancer cells, infected cells, immune cells involved in autoimmune disease, etc. by systemic administration of cytokine, e.g. intramuscular, intraperitoneal, intravenous, and the like.

In certain aspects, the method for treating and/or preventing disease can involve the steps of: (i) isolating an immune cell-containing sample; (ii) transducing or transfecting such cells with a nucleic acid sequence or vector, e.g., expressing a chimeric cytokine receptor; (iii) administering or infusing the cells from (ii) to the subject; and (iv) administering a cytokine that stimulates the infused cells. In certain aspects, the subject has undergone an immuno-depletion treatment prior to administering or infusing the cells to the subject. In certain aspects, the subject has not undergone an immuno-depletion treatment prior to administering or infusion the cells to the subject. In certain aspects, the subject has undergone an immuno-depletion treatment reduced in severity, without the use of the chimeric receptors described herein prior to administering or infusing the cells to the subject.

The immune cell-containing sample can be isolated from a subject or from other sources, for example as described above. The immune cells can be isolated from a subject's own peripheral blood (1st party), or in the setting of a hematopoietic stem cell transplant from donor peripheral blood (2nd party), or peripheral blood from an unconnected donor (3rd party). The immune cells can also be isolated from tumor tissue or other tissues in the body.

In some embodiments, the immune cells are contacted with the orthologous cytokine in vivo, i.e., where the immune cells are transferred to a recipient, and an effective dose of the orthologous cytokine is administered to the recipient and allowed to contact the immune cells in their native location, e.g. in lymph nodes, etc. In some embodiments, the contacting is performed in vitro. Where the cells are contacted with the orthologous cytokine in vitro, the cytokine is added to the cells in a dose and for a period of time sufficient to activate signaling from the receptor, which can utilize aspects of the native cellular machinery, e.g. accessory proteins, co-receptors, etc. The activated cells can be used for any purpose, including, but not limited to, experimental purposes relating to determination of antigen specificity, cytokine profiling, and for delivery in vivo.

In certain aspects, a therapeutically effective number of cells are administered to the subject. In certain aspects, the subject is administered or infused with cells expressing chimeric cytokine receptors on a plurality of separate occasions. In certain embodiments, at least 1×10⁶ cells/kg, at least 1×10⁷ cells/kg, at least 1×10⁸ cells/kg, at least 1×10⁹ cells/kg, at least 1×10¹⁰ cells/kg, or more are administered, sometimes being limited by the number of cells, e.g., T cells, obtained during collection. The transfected cells may be infused to the subject in any physiologically acceptable medium, normally intravascularly, although they may also be introduced into any other convenient site, where the cells may find an appropriate site for growth.

In certain aspects, the methods described herein comprise administering a therapeutically effective amount of cytokine to the subject. In certain aspects, the subject is administered the cytokine on a plurality of separate occasions. In certain aspects, the amount of cytokine that is administered, is an amount sufficient to achieve a therapeutically desired result (e.g., reduce symptoms of a disease in a subject). In certain aspects, the amount of cytokine that is administered is an amount sufficient to stimulate cell cycle progression, proliferation, viability and/or functional activity of a cell expressing a chimeric cytokine receptor described herein. In certain aspects, the cytokine is administered at a dose and/or duration that would is necessary to achieve a therapeutically desired result. In certain aspects, the cytokine is administered at a dose and/or duration sufficient to stimulate cell cycle progression, proliferation, viability and/or functional activity of a cell expressing a chimeric cytokine receptor described herein. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., for systemic administration, e.g., intramuscular, intraperitoneal, intravascular, and the like.

Indications for Adoptive Cell Transfer

The present disclosure provides a cell expressing a chimeric cytokine receptor described herein for use in treating and/or preventing a disease. The invention also relates to the use of a cell expressing a chimeric cytokine receptor described herein in the manufacture of a medicament for the treatment and/or prevention of a disease.

The disease to be treated and/or prevented by the methods of the present invention can be a cancerous disease, such as, but not limited to, bile duct cancer, bladder cancer, breast cancer, cervical cancer, ovarian cancer, colon cancer, endometrial cancer, hematologic malignancies, kidney cancer (renal cell), leukemia, lymphoma, lung cancer, melanoma, non-Hodgkin lymphoma, pancreatic cancer, prostate cancer, sarcoma and thyroid cancer.

The disease to be treated and/or prevented can be an autoimmune disease. Autoimmune diseases are characterized by T and B lymphocytes or other immune cell types that aberrantly target self-proteins, polypeptides, peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body. Autoimmune diseases include, but are not limited to, Type 1 diabetes, Rheumatoid arthritis, systemic lupus erythematosus, autoimmune thyroid diseases and Graves' disease.

The disease to be treated and/or prevented can be an inflammatory condition, such as cardiac fibrosis. In general, inflammatory conditions or disorders typically result in the immune system attacking the body's own cells or tissues and may cause abnormal inflammation, which can result in chronic pain, redness, swelling, stiffness, and damage to normal tissues. Inflammatory conditions are characterized by or caused by inflammation and include, but are not limited to, celiac disease, vasculitis, lupus, chronic obstructive pulmonary disease (COPD), irritable bowel disease, atherosclerosis, arthritis, myositis, scleroderma, gout, Sjorgren's syndrome, ankylosing spondylitis, antiphospholipid antibody syndrome, and psoriasis.

In certain embodiments, the chimeric cytokine receptors described herein are used to prevent and treat graft rejection. In certain aspects, the disease to be treated and/or prevented is allograft rejection. In certain aspects, the allograft rejection is acute allograft rejection.

In certain embodiments, the method is used to treat an infectious disease.

The disease to be treated and/or prevented can involve the transplantation of cells, tissues, organs or other anatomical structures to an affected individual. The cells, tissues, organs or other anatomical structures can be from the same individual (autologous or “auto” transplantation) or from a different individual (allogeneic or “allo” transplantation). The cells, tissues, organs or other anatomical structures can also be produced using in vitro methods, including cell cloning, induced cell differentiation, or fabrication with synthetic biomaterials.

Treatment can be combined with other active agents, such as, but not limited to, antibiotics, anti-cancer agents, anti-viral agents, and other immune modulating agents (e.g., antibodies against the Programmed Cell Death Protein-1 [PD-1] pathway or antibodies against Cytotoxic T Lymphocyte-associated Antigen-4 [CTLA-4]). Additional cytokines may also be included, e.g., interferon 7, tumor necrosis factor α, interleukin 12, etc.

### Pharmaceutical Compositions of the Invention

The present invention also relates to pharmaceutical compositions containing a plurality of cells expressing the chimeric cytokine receptor(s) described herein and/or the cytokines described herein. The cells and cytokines of the invention can be formulated in pharmaceutical compositions. These compositions can comprise, in addition to one or more of cytokines or cells expressing the chimeric cytokine receptor(s), a pharmaceutically acceptable excipient, carrier, buffer, stabilizer or other materials well known to those skilled in the art. Such materials should be non-toxic and should not interfere with the efficacy of the active ingredient. The pharmaceutical composition may optionally comprise one or more further pharmaceutically active polypeptides and/or compounds. Such a formulation may, for example, be in a form suitable for intravenous infusion.

For cytokines and cells expressing the chimeric receptors described herein that are to be given to an individual, administration is preferably in a “therapeutically effective amount” that is sufficient to show benefit to the individual. A “prophylactically effective amount” can also be administered, when sufficient to show benefit to the individual. The actual amount of cytokine or number of cells administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Examples

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques, cell culture, adoptive cell transfer, and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Methods

Primary cells and cell lines: The lentiviral packaging cell line HEK293T/17 (ATCC) was cultured in DMEM containing 10% fetal bovine serum and penicillin/streptomycin. BAF3-IL-2Rβ cells were previously generated by stable transfection of the human IL-2Rβ subunit into the BAF3 cell line, and were grown in RPMI-1640 containing 10% fetal bovine serum, penicillin, streptomycin and 100 IU/ml human IL-2 (hIL-2) (PROLEUKIN®, Novartis Pharmaceuticals Canada). The 32D-IL-2Rβ cell line was previously generated by stable transfection of the human IL-2Rβ subunit into the 32D cell line, and was grown in RPMI-1640 containing 10% fetal bovine serum, penicillin, streptomycin and 300 IU/ml hIL-2, or other cytokines, as indicated. Human PBMC-derived T cells (Hemacare) were grown in TexMACS™ Medium (Miltenyi Biotec, 130-097-196), containing 3% Human AB Serum (Sigma-Aldrich, H4522), and 300 IU/ml hIL-2, or other cytokines, as indicated. Human tumor-associated lymphocytes (TAL) were generated by culture of primary ascites samples for 14 days in T cell medium (a 50:50 mixture of the following: 1) RPMI-1640 containing 10% fetal bovine serum, 50 uM β-mercaptoethanol, 10 mM HEPES, 2 mM L-glutamine, penicillin, streptomycin; and 2) AIM V™ Medium (ThermoFisher, 12055083) containing a final concentration of 3000 IU/ml hIL-2. Following this high-dose IL-2 expansion, TAL were cultured in T cell medium containing 300 IU/ml hIL-2 or other cytokines, as indicated. The retroviral packaging cell line Platinum-E (Cell Biolabs, RV-101) was cultured in DMEM containing 10% FBS, penicillin/streptomycin, puromycin (1 mcg/ml), and blasticidin (10 mcg/ml).

Lentiviral production and transduction of 32D-IL-2Rβ cells: Chimeric receptor constructs were cloned into a lentiviral transfer plasmid and the resulting sequences were verified by Sanger sequencing. The transfer plasmid and lentiviral packaging plasmids were co-transfected into HEK293T/17 cells using the calcium phosphate transfection method as follows: Cells were plated overnight, and medium changed 2-4 hours prior to transfection. Plasmid DNA and water were mixed in a polypropylene tube, and CaCl₂ (0.25 M) was added dropwise. After a 2 to 5 minute incubation, the DNA was precipitated by mixing 1:1 with 2×HEPES-buffered saline (0.28M NaCl, 1.5 mM Na₂HPO₄, 0.1M HEPES). The precipitated DNA mixture was added onto the cells, which were incubated overnight at 37° C., 5% CO₂. The following day the HEK293T/17 medium was changed, and the cells were incubated for another 24 hours. The following morning, the cellular supernatant was collected from the plates, centrifuged briefly to remove debris, and the supernatant was filtered through a 0.45 micron filter. The supernatant was spun for 90 minutes at 25,000 rpm with a SW-32Ti rotor in a Beckman Optima L-XP Ultracentrifuge. The supernatant was removed and the pellet was resuspended in a suitable volume of Opti-MEM medium. The viral titer was determined by adding serial dilutions of virus onto BAF3-IL-2Rβ cells. At 48-72 hours after transduction, the cells were incubated with an anti-human G-CSFR APC-conjugated antibody (1:50; Miltenyi Biotec, 130-097-308) and Fixable Viability Dye eFluor™ 450 (1:1000, eBioscience™, 65-0863-14) for 15 minutes at 4° C., washed, and analyzed on a Cytek Aurora or BD FACS Calibur flow cytometer. Using the estimated titer determined by this method, the 32D-IL-2Rβ cell line was transduced with the lentiviral supernatant encoding the chimeric receptor construct at a Multiplicity of Infection (MOI) of 0.5. Transduction was performed by adding the relevant amount of viral supernatant to the cells, incubating for 24 hours, and then replacing the medium. At 3-4 days after transduction, the expression of human G-CSFR was determined by flow cytometry, as described above.

Lentiviral transduction of human primary T cells: For transduction of PBMC-derived T cells and TAL, cells were thawed and plated in the presence of Human T Cell TransAct™ (Miltenyi Biotec, 130-111-160), according to the manufacturer's guidelines. 24 hours after activation, lentiviral supernatant was added, at MOI of 0.125-0.5. 48 hours after activation, the cells split into fresh medium, to remove residual virus and activation reagent. Two to four days after transduction, the transduction efficiency was determined by flow cytometry, as described above. For experiments in which the transduction efficiency of CD4+ and CD8+ fractions was determined separately, antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336) were utilized, along with Fixable Viability Dye eFluor™ 450 (1:1000).

Human T cell and 32D-IL-2Rβ expansion assays: Human primary T cells or 32-IL-2Rβ cells expressing the indicated chimeric receptor constructs, generated above, were washed three times in PBS and re-plated in fresh medium, or had their medium gradually changed, as indicated. Complete medium was changed to contain either wild-type human G-CSF (generated in-house or NEUPOGEN®, Amgen Canada), mutant G-CSF (generated in-house), hIL-2, or no cytokine. Every 3-5 days, cell viability and density were determined by Trypan Blue exclusion, and fold expansion was calculated relative to the starting cell number. G-CSFR expression was assessed by flow cytometry as described above.

CD4+ and CD8+ human TAL expansion assay: To examine the expansion of the CD4+ and CD8+ fractions of TAL, ex vivo ascites samples were thawed, and the CD4+ and CD8+ fractions were enriched using the Human CD4+ T Cell Isolation Kit (Miltenyi Biotec, 130-096-533) and Human CD8+ T Cell Isolation Kit (Miltenyi Biotec, 130-096-495), respectively. After expansion in cytokine-containing medium, the immunophenotype of the cells was assessed by flow cytometry, utilizing antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336), along with Fixable Viability Dye eFluor™ 450 (1:1000).

Primary human T cell immunophenotyping assay: After expansion in cytokine-containing medium, the immunophenotype of T cells was assessed by flow cytometry, utilizing antibodies against human G-CSFR, CD4 (1:100, Alexa Fluor® 700 conjugate, BioLegend, 300526 or PE conjugate, eBioscience™, 12-0048-42, or Brilliant Violet 570™ conjugate, Biolegend, 317445), CD8 (1:100, PerCP conjugate, BioLegend, 301030), CD3 (1:100, Brilliant Violet 510™ or Brilliant Violet 750™ conjugate, BioLegend, 300448 or 344845), CD56 (1:100, Brilliant Violet 711™ conjugate, BioLegend, 318336), CCR7 (1:50, APC/Fire™ 750 conjugate, Biolegend, 353246), CD62L (1:33, PE/Dazzle™ 594 conjugate, Biolegend, 304842), CD45RA (1:33, FITC conjugate, Biolegend, 304148), CD45RO (1:25, PerCP-eFluor® 710 conjugate, eBioscience™, 46-0457-42), CD95 (1:33, PE-Cyanine7 conjugate, eBioscience™, 25-0959-42), along with Fixable Viability Dye eFluor™ 450 or 5106 (1:1000).

Retroviral transduction: The pMIG transfer plasmid (plasmid #9044, Addgene) was altered by restriction endonuclease cloning to remove the IRES-GFP (BglII to PacI sites), and introduce annealed primers encoding a custom multiple cloning site. The chimeric receptor constructs were cloned into the customized transfer plasmid and the resulting sequences were verified by Sanger sequencing. The transfer plasmid was transfected into Platinum-E cells using the calcium phosphate transfection method, as described above. 24 hours after transfection the medium was changed to 5 ml fresh complete medium. 48 hours after transfection the cellular supernatant was collected from the plates and filtered through a 0.45 micron filter. Hexadimethrine bromide (1.6 mcg/ml, Sigma-Aldrich) and murine IL-2 (2 ng/ml, Peprotech) were added to the supernatant. This purified retroviral supernatant was used to transduce murine lymphocytes as described below.

48 hours prior to collection of retroviral supernatant, 24-well adherent plates were coated with unconjugated anti-murine CD3 (5 mcg/ml, BD Biosciences, 553058) and anti-murine CD28 (1 mcg/ml, BD Biosciences, 553294) antibodies, diluted in PBS, and stored at 4 degrees Celsius. 24 hours prior to collection of retroviral supernatant, C57Bl/6J mice (generated in-house) were euthanized under an approved Animal Use Protocol administered by the University of Victoria Animal Care Committee. Spleens were harvested and murine T cells were isolated as follows: Spleens were manually dissociated and filtered through a 100 micron filter. Red blood cells were lysed by incubation in ACK lysis buffer (Gibco, A1049201) for five minutes at room temperature, followed by one wash in serum-containing medium. CD8a-positive or Pan-T cells were isolated using specific bead-based isolation kits (Miltenyi Biotec, 130-104-075 or 130-095-130, respectively). Cells were added to plates coated with anti-CD3- and anti-CD28-antibodies in murine T cell expansion medium (RPMI-1640 containing 10% FBS, penicillin/streptomycin, 0.05 mM β-mercaptoethanol, and 2 ng/ml murine IL-2 (Peprotech, 212-12) or 300 IU/mL human IL-2 (Proleukin), and incubated at 37 degrees Celsius, 5% CO₂ for 24 hours. On the day of transduction, approximately half of the medium was replaced with retroviral supernatant, generated above. Cells were spinfected with the retroviral supernatant at 1000 g, for 90 minutes, at 30 degrees Celsius. The plates were returned to the incubator for 0-4 hours, and then approximately half of the medium was replaced with fresh T cell expansion medium. The retroviral transduction was repeated 24 hours later, as described above, for a total of two transductions. 24 hours after the final transduction, the T cells were split into 6-well plates and removed from antibody stimulation.

48-72 hours after transduction, the transduction efficiency was assessed by flow cytometry to detect human G-CSFR, CD4 (Alexa Fluor 532 conjugate, eBioscience™, 58-0042-82), CD8a (PerCP-eFluor 710 conjugate, eBioscience™, 46-0081-82) and Fixable Viability Dye eFluor™ 450 (1:1000 dilution), as described above.

BrdU incorporation assay: Human primary T cells, 32D-IL-2Rβ cells, or murine primary T cells, generated as described above, were washed three times in PBS, and re-plated in fresh medium containing the relevant assay cytokine: no cytokine, hIL-2 (300 IU/ml), wildtype or engineered G-CSF (at concentrations indicated in individual experiments) for 48 hours. The BrdU assay procedure followed the instruction manual for the BD Pharmingen™ APC BrdU Flow Kit (BD Biosciences, 557892), with the following additions: Cells were co-incubated with BrdU and Fixable Viability Dye eFluor™ 450 (1:5000) for 30 minutes to 4 hours at 37 degrees Celsius. Flow cytometry was performed using a Cytek Aurora instrument. To specifically assess the proliferation of murine T cells expressing chimeric receptors, additional staining for human G-CSFR (1:20 dilution), CD4 (1:50 dilution) and CD8 (1:50 dilution) was performed for 15 minutes on ice, prior to fixation.

Western blots: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml), wildtype G-CSF (at concentrations indicated in individual experiments), or G-CSF₁₃₇ (30 ng/ml) for 20 minutes at 37 degrees Celsius. Cells were washed once in a buffer containing 10 mM HEPES, pH7.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% NP-40 (Sigma) for 10 minutes on ice. The lysate was centrifuged for 10 minutes at 13,000 rpm at 4 degrees Celsius, and the supernatant (cytoplasmic fraction) was collected. The pellet (containing nuclear proteins) was resuspended in the wash buffer above, with the addition of 0.42M NaCl and 20% glycerol. Nuclei were incubated for 30 minutes on ice, with frequent vortexing, and the supernatant (nuclear fraction) was collected after centrifuging for 20 minutes at 13,000 rpm at 4 degrees Celsius. The cytoplasmic and nuclear fractions were reduced (70 degrees Celsius) for 10 minutes and run on a NuPAGE™ 4-12% Bis-Tris Protein Gel. The gels were transferred to nitrocellulose membrane (60 min at 20V in a Trans-Blot® SD Semi-Dry Transfer Cell), dried, and blocked for 1 hr in Odyssey® Blocking Buffer in TBS (927-50000). The blots were incubated with primary antibodies (1:1000) overnight at 4 degrees Celsius in Odyssey® Blocking Buffer in TBS containing 0.1% Tween20. The primary antibodies utilized were obtained from Cell Signaling Technologies: Phospho-Jak1 (Tyr1034/1035) (D7N4Z) Rabbit mAb #74129, Phospho-Jak2 (Tyr1007/1008) #3771, Phospho-Jak3 (Tyr980/981) (D44E3) Rabbit mAb #5031, Phospho-p70 S6 Kinase (Thr421/Ser424) Antibody #9204, Phospho-Shc (Tyr239/240) Antibody #2434, Phospho-Akt (Ser473) (D9E) XP® Rabbit mAb #4060, Phospho-S6 Ribosomal Protein (Ser235/236) Antibody #2211, Phospho-p44/42 MAPK (Erk1/2) (Thr202/Tyr204) Antibody #9101, 3-Actin (13E5) Rabbit mAb #4970, Phospho-STAT1 (Tyr701) (58D6) Rabbit mAb #9167, Phospho-STAT3 (Tyr705) (D3A7) XP® Rabbit mAb #9145, Phospho-STAT4 (Tyr693) Antibody #5267, Phospho-STAT5 (Tyr694) (C11C5) Rabbit mAb #9359, and Histone H3 (96C10) Mouse mAb #3638. Blots were washed three times in TBS containing 0.1% Tween20 and incubated with secondary antibodies (1:10,000) in TBS buffer containing 0.1% Tween20 for 30-60 minutes at room temperature. The secondary antibodies obtained from Cell Signaling Technologies were Anti-mouse IgG (H+L) (DyLight™ 800 4×PEG Conjugate) #5257 and Anti-rabbit IgG (H+L) (DyLight™ 800 4×PEG Conjugate) #5151. Blots were washed and exposed on a LI-COR Odyssey imager.

Flow cytometry to detect phosphorylated proteins: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml) or wild-type G-CSF (100 ng/ml) for 20 minutes at 37 degrees Celsius, in the presence of Fixable Viability Dye eFluor™ 450 (1:1000), and anti-G-CSFR (1:20), anti-CD4 (1:50) and anti-CD8a (1:50), as indicated. Cells were pelleted and fixed with BD Phosflow™ Fix Buffer I (BD Biosciences, 557870) for 15 minutes at room temperature. Cells were washed, then permeabilized using BD Phosflow™ Perm Buffer III (BD Biosciences, 558050) on ice for 15 minutes. Cells were washed twice and resuspended in buffer containing 20 ul of BD Phosflow™ PE Mouse Anti-Stat3 (pY705) (BD Biosciences, 612569) or PE Mouse IgG2a, κ Isotype Control (BD Biosciences, 558595). Cells were washed and flow cytometry was performed using a Cytek Aurora instrument.

### Example 1: Expansion of Human T Cells Expressing G2R-1, G-CSFR/IL-2Rβ Subunit Alone, the Myc-Tagged G-CSFR/γc Subunit Alone, or the Full-Length G-CSFR

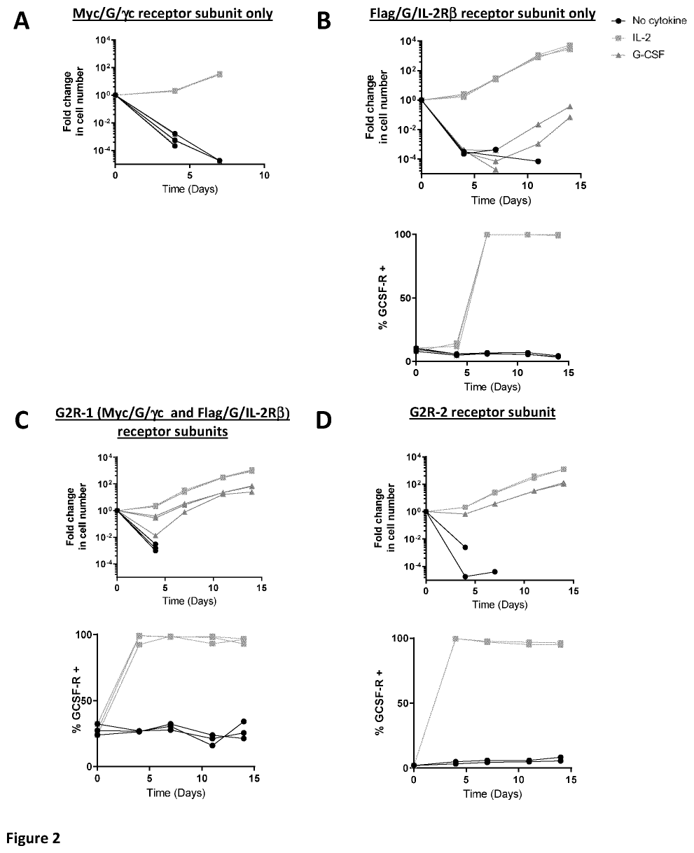

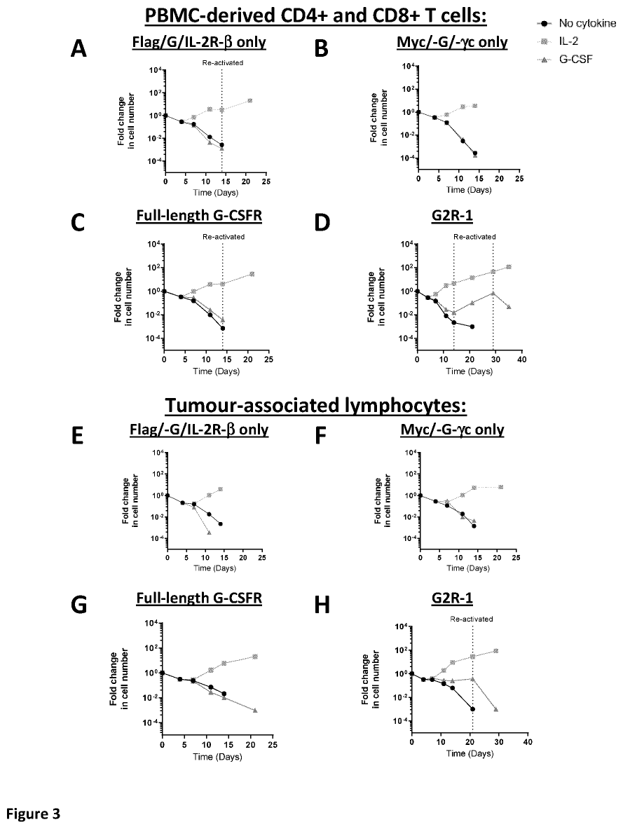

PBMC-derived T cells or tumour-associated lymphocytes (TAL) were transduced with lentiviruses encoding the chimeric receptor constructs shown in FIG. 1, and cells were washed and re-plated in the indicated cytokine. Cells were counted every 3-4 days. G/γc was tagged at its N-terminus with a Myc epitope (Myc/G/γc), and G/IL-2Rβ was tagged at its N-terminus with a Flag epitope (Flag/G/IL-2Rβ); these epitope tags aid detection by flow cytometry and do not impact the function of the receptors. As expected, all T cell cultures showed proliferation in response to the positive control cytokine, IL-2 (300 IU/ml). After stimulation with G-CSF (100 ng/ml), proliferation was observed only for PMBC-derived T cells and TAL expressing the G2R-1 chimeric cytokine receptor (FIG. 3). Note that the lentiviral transduction efficiency was less than 100% such that less than 100% of T cells expressed the indicated chimeric cytokine receptors, which likely accounts for the lower rate of proliferation mediated by G2R-1 relative to IL-2. Similarly, increased proliferation was observed in 32D-IL-2Rβ cells (stably expressing the human IL-2Rβ subunit) expressing G-CSFR chimeric receptor subunits G2R-1 and G2R-2 and stimulated with G-CSF (FIG. 2). In contrast to T cells, 32D-IL-2Rβ cells expressing the G/IL-2Rβ chimeric receptor subunit alone proliferated in response G-CSF (FIG. 2); G-CSF-induced proliferation was not seen in 32D-IL-2Rβ cells expressing the G/γc chimeric receptor subunit alone (FIG. 2).

These results show that G-CSF is capable of stimulating proliferation and viability of PMBC-derived T cells and TALs expressing the G2R-1 chimeric receptors and of 32D-IL-2Rβ cells expressing the G/IL-2Rβ, G2R-1 and G2R-2 chimeric receptors.

### Example 2: G-CSFR ECD is Expressed on the Surface of Cells Transduced with G/IL-2RD, G2R-1 and G2R-2

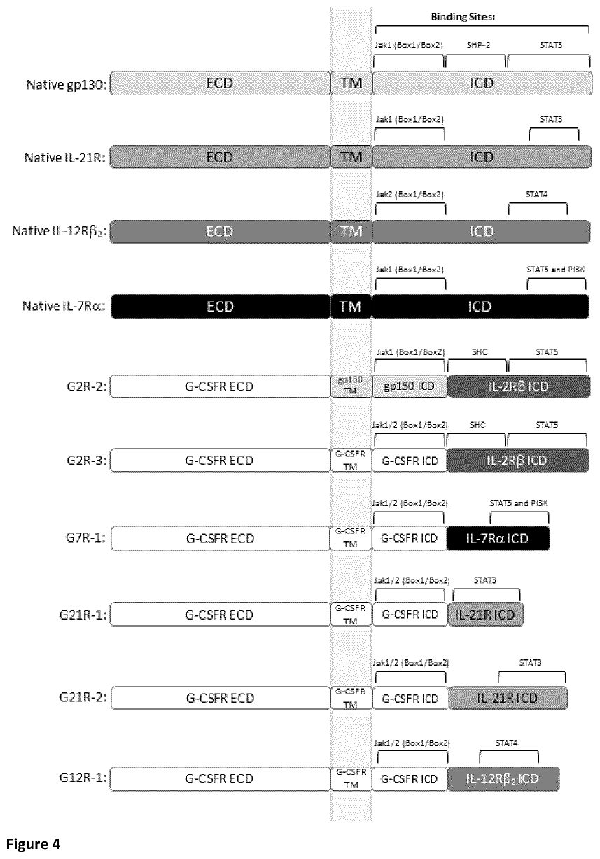

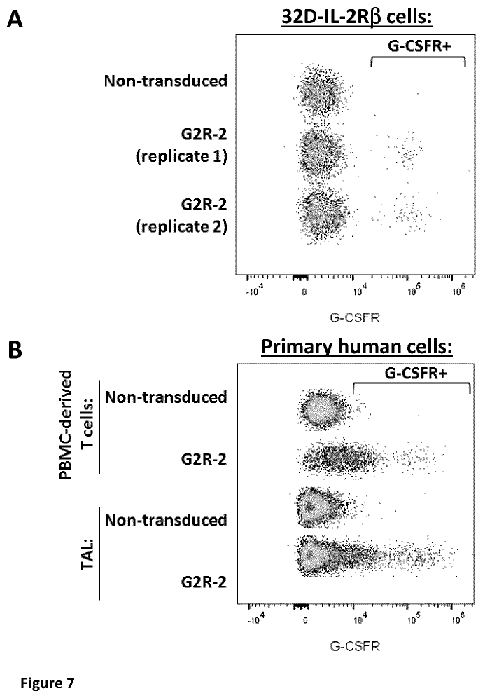

Flow cytometry was performed on the 32D-IL-2Rβ cell line, PBMC-derived human T cells and human tumour-associated lymphocytes after transduction with a lentiviral vector encoding the G2R-2 chimeric cytokine receptor (shown schematically in FIGS. 4 and 6) to determine if the cells express the G-CSFR ECD on the cell surface. G-CSFR positive cells were detected in all transduced cell types (FIG. 7). In a separate experiment, 32D-IL-2Rβ cells expressing the G/IL-2Rβ, G2R-1 and G2R-2 chimeric receptors were positive for the G-CSFR ECD by flow cytometry (lower panels in FIG. 2B-D).

These results indicate that the G/IL-2Rβ, G2R-1 and G2R-2 chimeric receptors are expressed on the cell surface.

### Example 3: Expansion of Cells Expressing G2R-2 Compared to Non-Transduced Cells

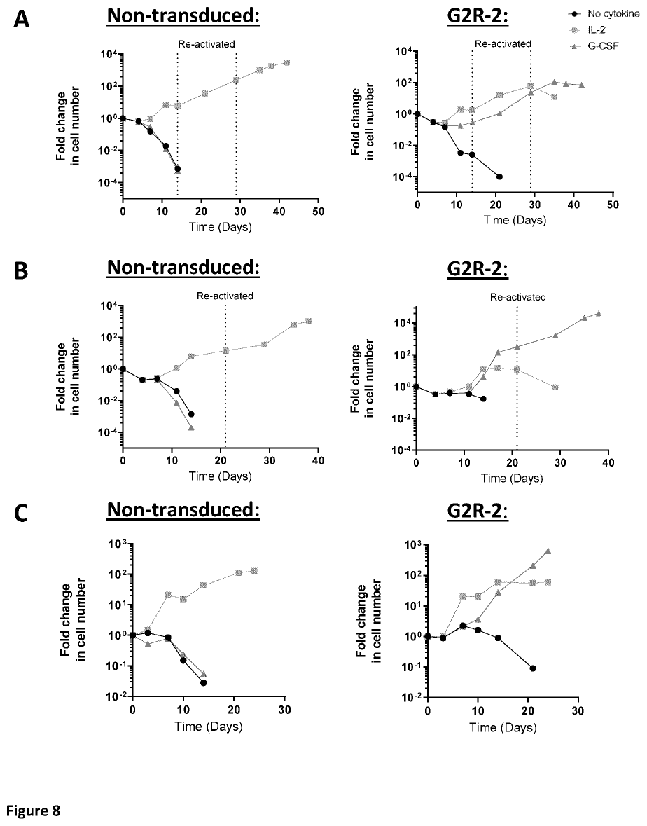

Human PBMC-derived T cells and human tumour-associated lymphocytes were lentivirally transduced with the G2R-2 receptor construct (FIGS. 4 and 6), washed, and re-plated with the indicated cytokine. In some experiments, T cells were also re-activated periodically by stimulation with TransAct reagent. Live cells were counted every 3-4 days. Proliferation of the PMBC-derived T cells (FIG. 8A) and tumour-associated lymphocytes (two independent experiments in FIG. 8B,C) was observed after stimulation with G-CSF (100 ng/ml) in cells expressing the G2R-2 chimeric receptor but not in non-transduced cells.

These results show that G-CSF-induced activation of the G2R-2 chimeric receptor is sufficient to induce proliferation and viability of immune cells.

### Example 4: Expansion and Immunophenotype of CD4- or CD8-Selected Human Tumour-Associated Lymphocytes Expressing G2R-2 Compared to Non-Transduced Cells

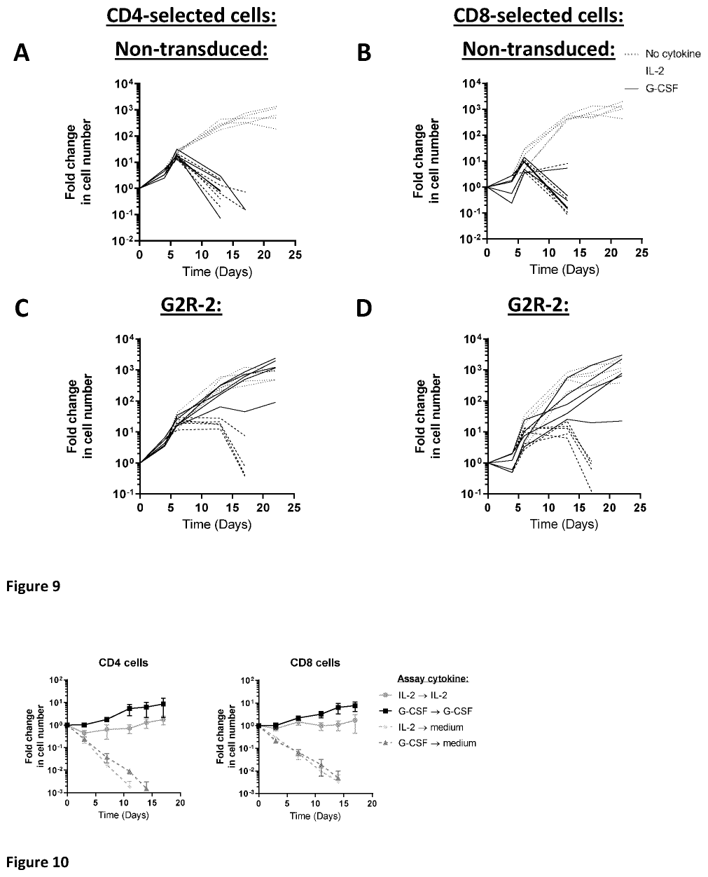

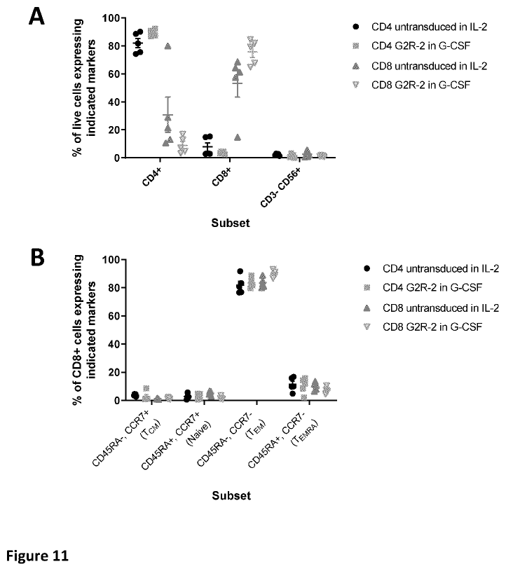

CD4-selected and CD8-selected human T cells were transduced with lentiviral vector encoding G2R-2 (FIG. 6), or left non-transduced where indicated. Cells were washed and re-plated with the indicated cytokine and counted every 3-4 days. Proliferation of CD4- or CD8-selected TALs expressing G2R-2 was observed after stimulation with G-CSF (100 ng/ml) or IL-2 (300 IU/ml), but not in the absence of added cytokine (medium alone) (FIGS. 9 and 10). In FIG. 9, each line represents results from one of 5 patient samples.

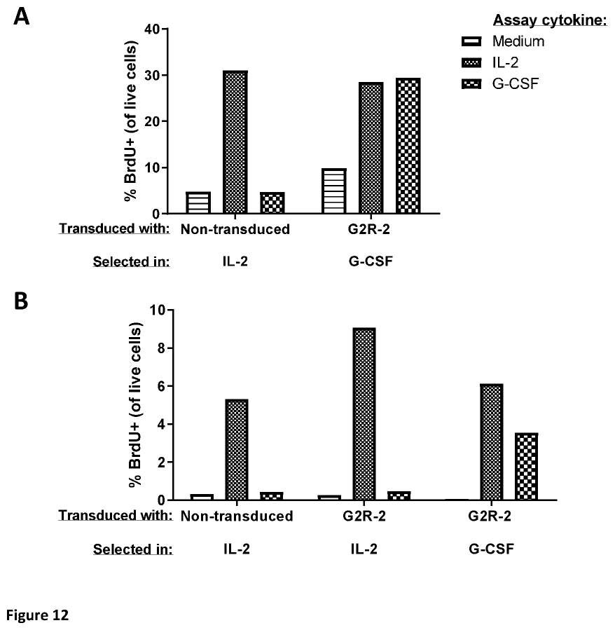

Immunophenotyping by flow cytometry demonstrated that T cells cultured in G-CSF or IL-2 retained their CD4+ or CD8+ identity (FIG. 11A), lacked an NK cell phenotype (CD3-CD56+) (FIG. 11A), and exhibited a CD45RA-CCR7− T effector memory (T_{EM}) phenotype under these culture conditions (FIG. 11B).

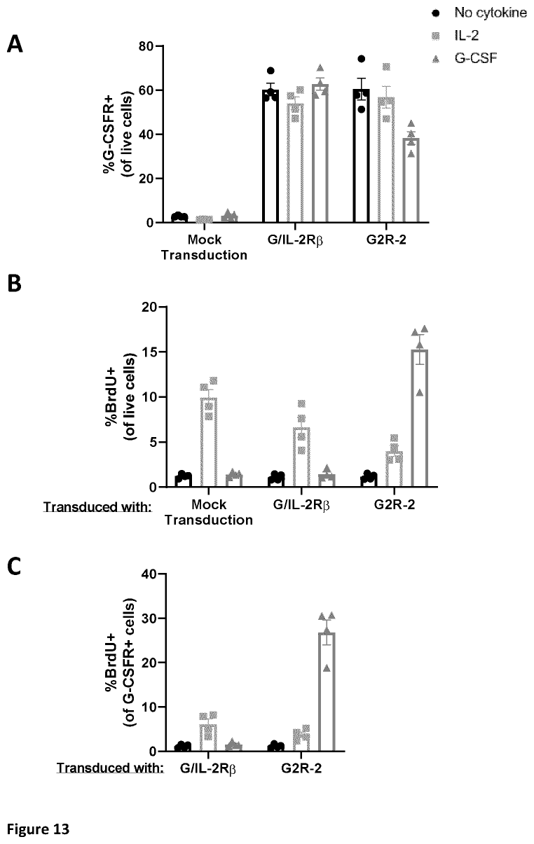

BrdU assays were performed to confirm increased cell cycle progression of T cells expressing G2R-2 upon stimulation with G-CSF (FIG. 12). T cells were selected by culture in IL-2 or G-CSF, as indicated, prior to the assay. Both tumour-associated lymphocytes (FIG. 12A) and PBMC-derived T cells (FIG. 12B) were assessed.

These results show that G-CSF can selectively activate cell cycle progression and long-term expansion of primary human TALs by activation of the chimeric cytokine receptor G2R-2. These results also indicate that activation of the G2R-2 chimeric receptor by homodimer formation is sufficient to activate cytokine-like signaling and proliferation in TALs. Furthermore, TALs expressing G2R-2 remain cytokine dependent in that they undergo cell death upon withdrawal of G-CSF, similar to the response to IL-2 withdrawal. TALs cultured in G-CSF maintain a similar immunophenotype as TALs cultured in IL-2.

### Example 5: Primary Murine T Cells Expressing G2R-2 Proliferate in Response to G-CSF

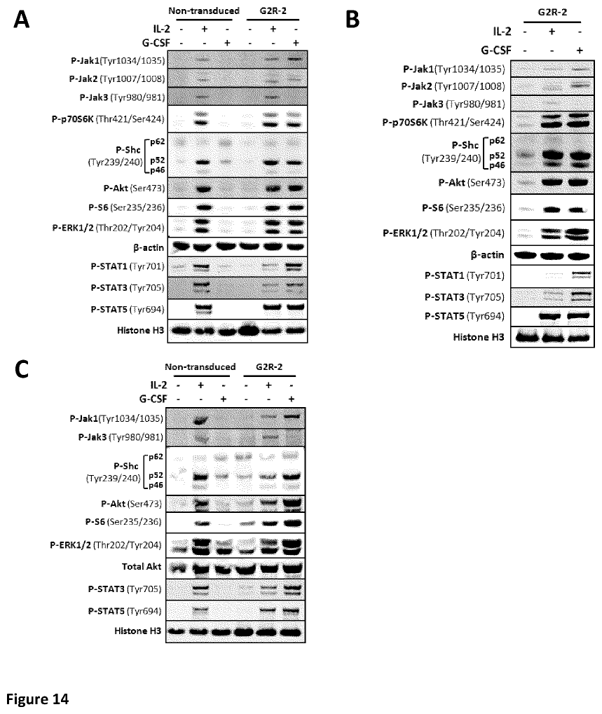

BrdU incorporation assays were performed to assess proliferation of primary murine T cells expressing G2R-2 or the single-chain G/IL-2Rβ (a component of G2R-1) versus mock-transduced cells upon stimulation with G-CSF. All cells were expanded in IL-2 for 3 days prior to assay. Cell surface expression of G2R-2 or G/IL-2Rβ was confirmed by flow cytometry (FIG. 13A). As indicated, cells were then plated in IL-2 (300 IU/ml), wildtype G-CSF (100 ng/ml) or no cytokine. Increased cell cycle progression after stimulation with G-CSF was observed in the cells expressing G2R-2 above that of non-transduced cells or cells expressing the single-chain G/IL-2Rβ (FIG. 13B,C). Panels B and C show results for all live cells or G-CSFR+ cells, respectively.

These results indicate that, in response to G-CSF-induced homodimerization, the G2R-2 chimeric receptor is more efficient than the single chain G/IL-2Rβ receptor to activate cytokine-like signaling and proliferation in murine T cells.

### Example 6: Activation of Cytokine-Associated Intracellular Signaling Events in Human Primary T Cells Expressing G2R-2 in Response to G-CSF or IL-2

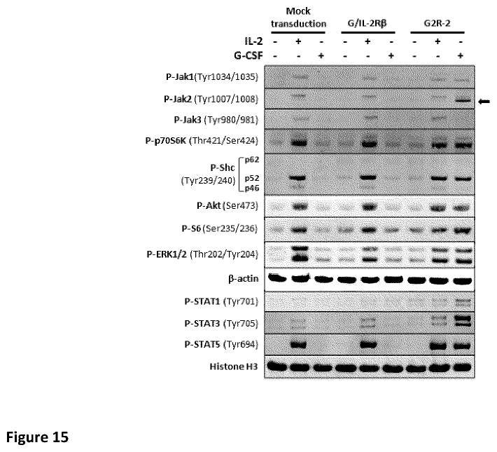

To confirm that the chimeric cytokine receptors were indeed capable of activating cytokine signaling similar to that of IL-2, the ability of the cytokine receptors to activate various signaling molecules was assessed. Tumour-associated lymphocytes and PBMC-derived T cells expressing G2R-2 were previously expanded in G-CSF, while non-transduced cells were previously expanded in IL-2. The cells were washed and then stimulated with IL-2 (300 IU/ml), wildtype G-CSF (100 ng/ml), or no cytokine, and western blots were performed on the cell lysates using antibodies directed against the indicated signaling molecules (FIG. 14). Panels A and B show results for TAL, and panel C for PBMC-derived T cells. T cells expressing G2R-2 activated IL-2-related signaling molecules upon stimulation with G-CSF to a similar extent as seen after IL-2 stimulation of non-transduced cells or transduced cells, with the expected exception that G-CSF induced Jak2 phosphorylation whereas IL-2 induced Jak3 phosphorylation.

These results confirm that the G2R-2 chimeric receptor is capable of activating IL-2 receptor-like cytokine receptor signaling upon stimulation with G-CSF.

### Example 7: Cytokine Signaling is Activated in Response to G-CSF in Murine Primary T Cells Expressing G2R-2

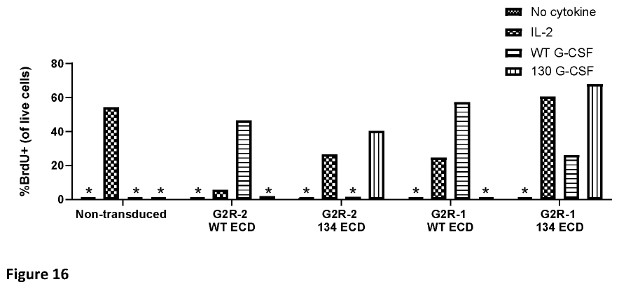

To assess whether the chimeric cytokine receptor G2R-2 or the single-chain G/IL-2Rβ (from G2R-1) were capable of activating cytokine signaling, the ability of these cytokine receptors to activate various signaling molecules was assessed by western blot of cell lysates of murine primary T cells expressing G2R-2 or G/IL-2Rβ versus mock-transduced cells. All cells were expanded in IL-2 for 3 days prior to assay. Cells were then washed and stimulated with IL-2 (300 IU/ml), wildtype G-CSF (100 ng/ml) or no cytokine. The cells expressing G2R-2 activated IL-2-related signaling molecules upon stimulation with G-CSF to a similar extent as seen after IL-2 stimulation of non-transduced cells or transduced cells, with the expected exception that G-CSF induced Jak2 phosphorylation whereas IL-2 induced Jak3 phosphorylation (FIG. 15). In contrast, G/IL-2Rβ did not activate cytokine signaling upon exposure to G-CSF.

These results confirm in primary murine T cells that the G2R-2 chimeric receptor is capable of activating IL-2 receptor-like cytokine receptor signaling by homodimerization upon G-CSF stimulation, whereas the single-chain G/IL-2Rβ alone is not capable of activating cytokine signaling by homodimerization in response to G-CSF.

### Example 8: Expression of Chimeric Receptors Leads to Proliferation of 32D-IL-2RD Cells and Primary Murine T Cells after Stimulation with an Orthogonal G-CSF

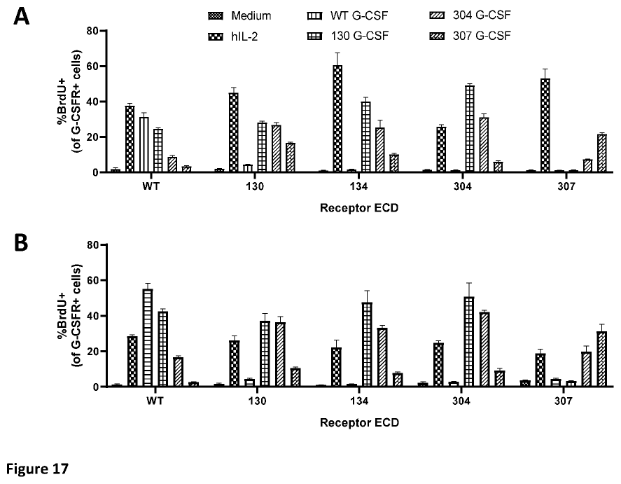

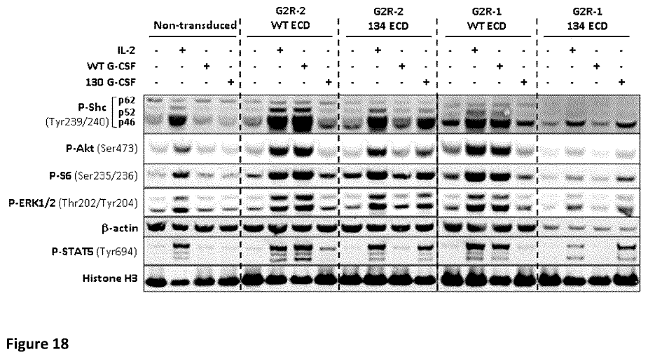

To determine if cells expressing chimeric cytokine receptors could be selectively activated in response to an orthogonal version of G-CSF, 32D-IL-2Rβ cells or primary murine T cells were transduced with the chimeric receptors G2R-1 and G2R-2 that comprises the wild-type G-CSFR ECD (G2R-1 WT ECD, G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2, wild type G-CSF or the orthogonal G-CSF (130 G-CSF) capable of binding to G2R-1 134 ECD and G2R-2 134 ECD, but with significantly reduced binding to wild-type G-CSFR. BrdU incorporation assays were performed to assess the ability of the cells to promote cell cycle progression upon cytokine stimulation (FIG. 16). 32D-IL-2Rβ cells expressing G2R-2 134 ECD demonstrated cell cycle progression upon stimulation with 130 G-CSF (harboring amino acid substitutions E46R, L108K, and D112R; 30 ng/ml), but did not undergo cell cycle progression upon stimulation with wild-type G-CSF (30 ng/ml). The orthogonal nature of engineered cytokine:receptor ECD pairs was further demonstrated by stimulating primary murine T cells in a “criss-cross” proliferation assay, where cells expressing G2R-3 (FIG. 4) with the WT, 130, 134, 304 or 307 ECD were stimulated with WT, 130, 304 or 307 cytokine (100 ng/ml) (FIG. 17). The 130 ECD harbors the amino acid substitutions: R41E, and R167D. The 304 ECD harbors the amino acid substitutions: R41E, E93K and R167D; whereas the 304 cytokine harbors the amino acid substitutions: E46R, L108K, D112R and R147E. The 307 ECD harbors the amino acid substitutions: R41E, D197K, D200K and R288E; whereas the 307 cytokine harbors the amino acid substitutions: S12E, K16D, E19K and E46R. Panels A and B in FIG. 17 represent independent experimental replicates.

These results demonstrate that cells expressing an orthogonal chimeric cytokine receptor are capable of selective activation and cell cycle progression upon stimulation with an orthogonal G-CSF.

### Example 9: Intracellular Signaling is Activated in 32D-IL2Rβ Cells and Primary Human T Cells Expressing Orthogonal Chimeric Cytokine Receptors and Stimulated with an Orthogonal G-CSF

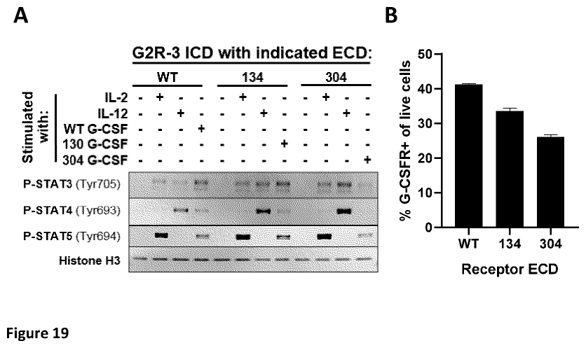

To determine if cells expressing chimeric cytokine receptors could selectively activate intracellular cytokine signaling events in response to an orthogonal version of G-CSF, 32D-IL-2RO cells were transduced with the chimeric receptors G2R-1 and G2R-2 that comprises the wild-type G-CSFR ECD (G2R-1 WT ECD and G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2 (300 IU/ml), wild type G-CSF (30 ng/ml) or the orthogonal G-CSF (130 G-CSF-E46R_L108K_D112R; 30 ng/ml) capable of binding to G2R-1 134 ECD, G2R-2 134 ECD, but with significantly reduced binding to wild-type G-CSFR. Western blots were performed on cell lysates to assess the ability of the cells to activate cytokine signaling upon exposure to cytokines (FIG. 18). Cells expressing G2R-2 134 ECD showed evidence of cytokine signaling upon stimulation with 130 G-CSF but not wild-type G-CSF. Furthermore, cells expressing G2R-2 WT ECD were not able to activate cytokine signaling upon stimulation with 130 G-CSF.

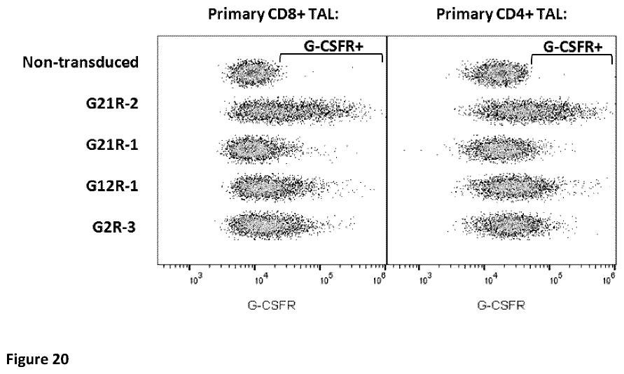

The orthogonal nature of engineered cytokine:receptor pairs was further demonstrated by subjecting primary murine T cells to western blot analysis, where cells expressing G2R-3 with the WT, 134, or 304 ECD (R41E_E93K_R167D) were stimulated with WT, 130, or 304 G-CSF (E46R_L108K_D112R_R147E; 100 ng/ml) and the indicated signaling events were measured (FIG. 19A). IL-2 (300 IU/ml) and IL-12 (10 ng/ml) served as control cytokines. Cells expressing G2R-3 WT ECD showed evidence of cytokine signaling upon stimulation with IL-2, IL-12 or WT G-CSF. Cells expressing G2R-3 134 ECD showed evidence of cytokine signaling upon stimulation with IL-2, IL-12 or 130 G-CSF. Cells expressing G2R-3 304 ECD showed evidence of cytokine signaling upon stimulation with IL-2, IL-12 or 304 G-CSF.

Cell surface expression of the three ECD variants of G2R-3 was confirmed by flow cytometry (FIG. 19B).

These results demonstrate that cells expressing an orthogonal chimeric cytokine receptor are capable of selective activation of intracellular cytokine signaling events upon stimulation with an orthogonal G-CSF.

### Example 10: Expression of G2R-3 Leads to Expansion, Cell Cycle Progression, and Cytokine-Related Intracellular Signaling and Immunophenotype in Primary Human T Cells

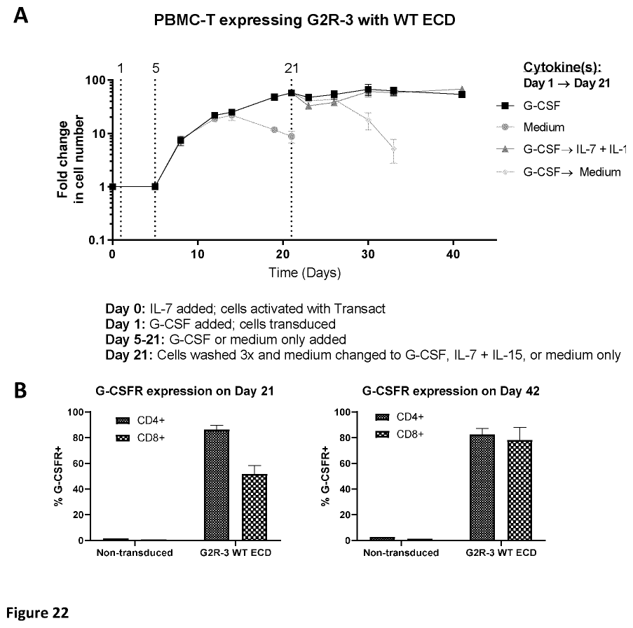

To determine whether the G2R-3 chimeric receptor can promote cytokine signaling-related events upon stimulation with G-CSF in primary human T cells, TALs were transduced with a lentiviral vector encoding G2R-3. T-cell expansion assays were performed to test the proliferation of cells upon stimulation with IL-2 (300 IU/ml), wildtype G-CSF (100 ng/ml) or no cytokine. Live cells were counted every 3-4 days. In contrast to their non-transduced counterparts, primary TALs expressing G2R-3 expanded in culture in response to G-CSF (FIG. 21A).

To determine whether cytokine signaling events were activated upon stimulation with G-CSF, western blots were performed on cell lysates to assess intracellular signaling. Cells were harvested from the expansion assay, washed, and stimulated with IL-2 (300 IU/ml) or wildtype G-CSF (100 ng/ml). Primary TALs expressing G2R-3 demonstrated IL-2-related signaling events in response to G-CSF, with the expected exception that G-CSF induced Jak2 phosphorylation whereas IL-2 induced Jak3 phosphorylation (FIG. 21B).

BrdU incorporation assays were performed to assess cell cycle progression upon stimulation with G-CSF. Cells were harvested from the expansion assay, washed, and re-plated in IL-2 (300 U/ml), wildtype G-CSF (100 ng/ml) or no cytokine. Primary TALs expressing G2R-3 demonstrated cell cycle progression in response to G-CSF (FIG. 21C).

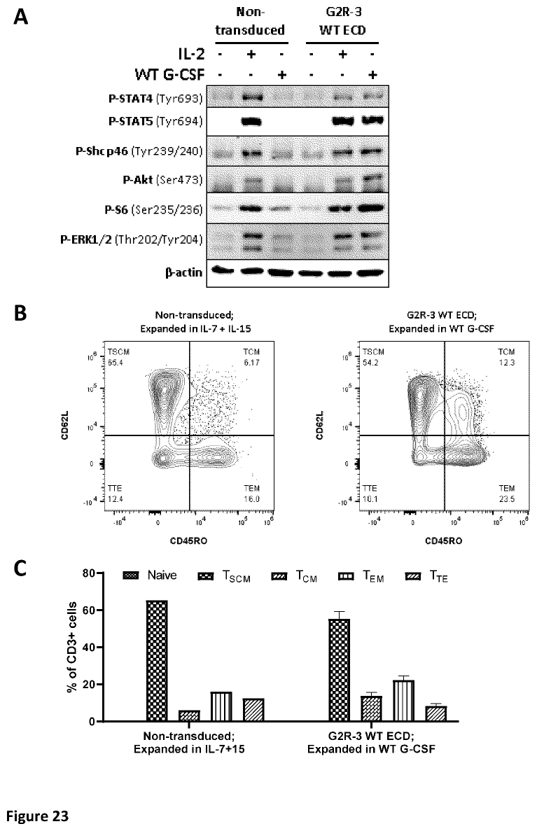

G-CSF-induced expansion of cells expressing G2R-3 was also demonstrated using primary PBMC-derived human T cells (FIG. 22). Cells expressing G2R-3 WT ECD expanded in response to WT G-CSF but not medium alone (FIG. 22A). To demonstrate the continued dependence of cells on exogenous cytokine, on Day 21 of culture, cells from the G-CSF-expanded condition were washed and re-plated in WT G-CSF (100 ng/mL), IL-7 (20 ng/mL)+IL-15 (20 ng/mL), or medium only. Only cells re-plated in the presence of G-CSF or IL-7+IL-15 remained viable over time.

Expression of G-CSFR ECD, as assessed by flow cytometry, remained stable on both CD4+ and CD8+ T cells between days 21-42 of expansion (FIG. 22B).

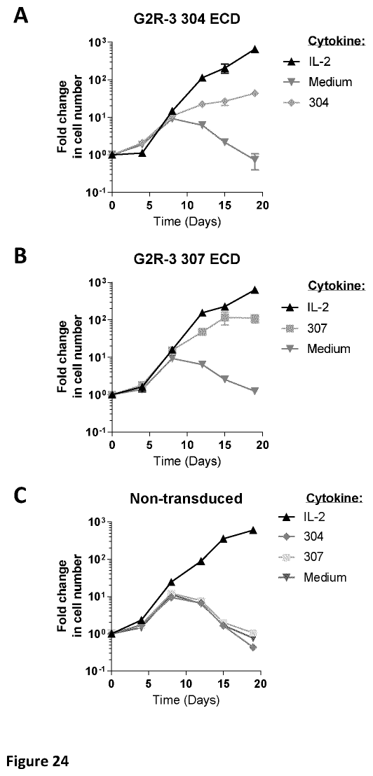

By western blot, primary PBMC-derived T cells expressing G2R-3 demonstrated IL-2-related signaling events in response to G-CSF (FIG. 23A).

Flow cytometry-based immunophenotyping was performed on primary PBMC-derived T cells expanded for 42 days in WT G-CSF versus IL-7+IL-15. Cells expressing G2R-3 WT ECD and cultured in G-CSF retained a similar phenotype to non-transduced cells cultured in IL-7+IL-15, with primarily a CD62L+, CD45RO+ phenotype, which is indicative of a stem cell-like memory T cell phenotype (T_{SCM}) (FIG. 23B, C). Likewise, the fractions of central memory (T_{CM}), effector memory (T_{EM}), and terminally differentiated (T_{TE}) T cells were similar.

These results confirm that the G2R-3 chimeric cytokine receptor is capable of activating cytokine signaling events and promoting cell cycle progression and expansion in primary cells. The immunophenotype of T cells expressing G2R-3 and expanded long-term in G-CSF is similar to non-transduced cells expanded in IL-7+IL-15.

### Example 11: Orthogonal G-CSF Induces Expansion and Proliferation in Primary Human T Cells Expressing G2R-3 with Orthogonal ECD

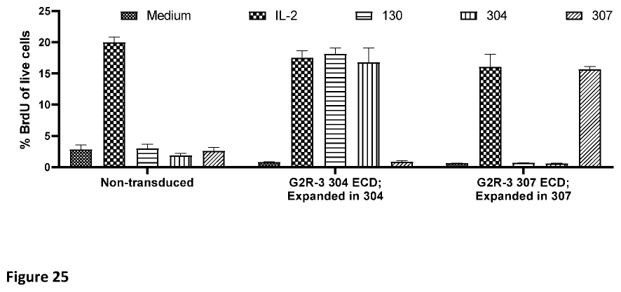

We assessed whether the chimeric cytokine receptor G2R-3 with 304 (R41E_E93K_R167D) or 307 (R41E_D197K_D200K_R288E) ECD was capable of inducing proliferation and expansion in response to stimulation with the orthogonal ligands 130, 304 or 307 (S12E_K16D_E19K_E46R) G-CSF, primary PBMC-derived human T cells were transduced with lentiviral vectors encoding G2R-3 304 ECD or G2R-3 307 (R41E_D197K_D200K_R288E) ECD. T-cell growth assays were performed to assess the fold expansion of cells when cultured with IL-2 (300 IU/ml), 304 G-CSF (100 ng/ml), 307 G-CSF (100 ng/ml) or no cytokine. Live cells were counted every 3-4 days. T cells expressing G2R-3 304 ECD expanded in culture in response to IL-2 or 304 G-CSF (FIG. 24A). T cells expressing G2R-3 307 ECD expanded in culture in response to IL-2 or 307 G-CSF (FIG. 24B). Non-transduced T cells only expanded in response to IL-2 (FIG. 24C).

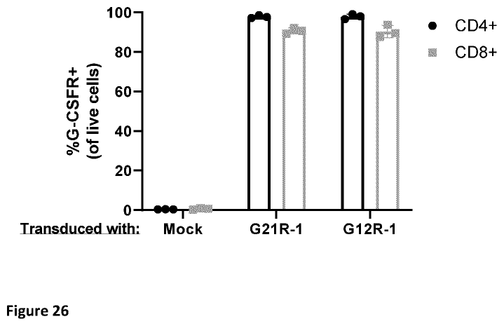

BrdU incorporation assays were performed to assess cell cycle progression upon stimulation with 130, 304 and 307 G-CSF in a criss-cross design. Cells were harvested from the expansion assay, washed, and re-plated in IL-2 (300 IU/ml), 130 G-CSF (100 ng/ml), 304 G-CSF (100 ng/ml), 307 G-CSF (100 ng/ml) or no cytokine. Primary human T cells expressing G2R-3 304 ECD demonstrated cell cycle progression in response to 130 or 304 G-CSF, but not in response to 307 G-CSF (FIG. 25). T cells expressing G2R-3 307 ECD demonstrated cell cycle progression in response to 307 G-CSF, but not in response to 130 or 304 G-CSF. All T cells demonstrated cell cycle progression in response to IL-2.

The results show that the chimeric receptors G2R-3 304 ECD and G2R-3 307 ECD are capable of inducing selective cell cycle progression and expansion of primary human CD4+ and CD8+ T cells upon stimulation with orthogonal 304 or 307 G-CSF, respectively. Furthermore, 130 G-CSF can stimulate proliferation of cells expressing G2R-3 304 ECD, but not G2R-3 307 ECD.

### Example 12: G-CSFR ECD is Expressed on the Surface of Primary Human Tumor-Associated Lymphocytes (TAL) Transduced with G21R-1, G21R-2, G12R-1 and G2R-3 Chimeric Receptor Constructs

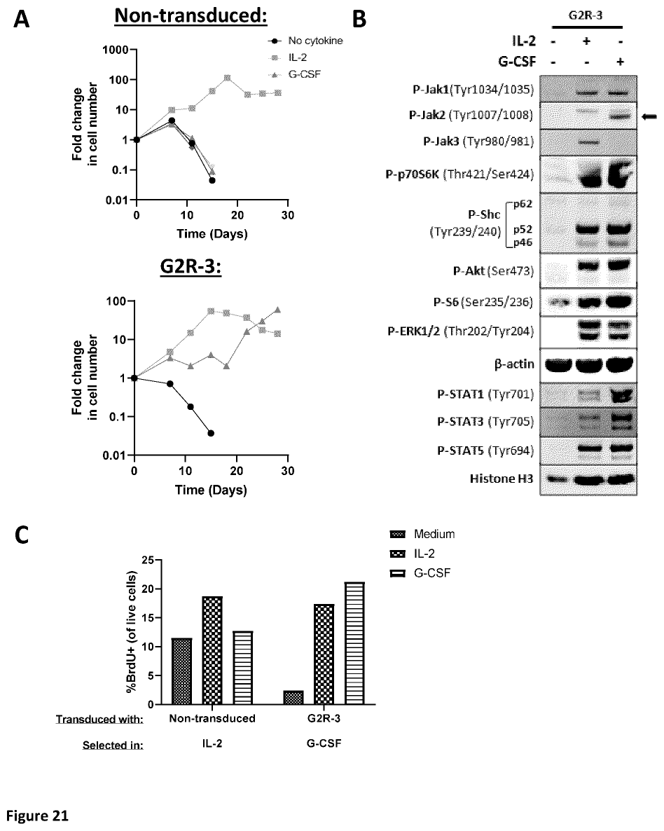

To assess whether chimeric cytokine receptor constructs can be expressed on the surface of primary human tumor-associated lymphocytes (TAL), TAL were transduced with lentiviral vectors encoding the G21R-1, G21R-2, G12R-1 and G2R-3 chimeric receptors, and the cells were tested by flow cytometry for G-CSFR ECD expression on the cell surface (FIG. 20). For all four chimeric cytokine receptor designs, G-CSFR ECD positive cells were detected.

These results demonstrate that the G21R-1, G21R-2, G12R-1 and G2R-3 chimeric receptors are capable of being expressed on the surface of primary cells. These results also indicate that G-CSFR ECD chimeric receptor designs are expressed on the surface of primary cells.

### Example 13: G-CSFR ECD is Expressed on the Surface of Primary Murine T Cells Transduced with G12R-1 and G21R-1 Chimeric Receptor Constructs

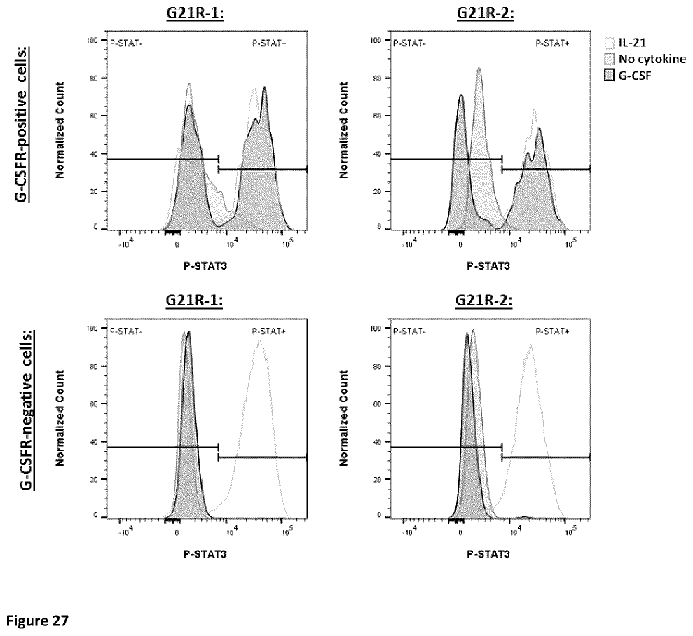

To determine if the G12R-1 and G21R-1 chimeric receptors are capable of being expressed on the surface of primary T cells, primary murine T cells were transduced with retroviral vectors encoding the G12R-1 and G21R-1 chimeric receptors and analyzed by flow cytometry (FIG. 26).

The results show that G-CSFR ECD is expressed on the surface of primary murine CD4+ and CD8+ T cells transduced with retroviral vectors encoding G12R-1 and G21R-1.

### Example 14: G-CSF Induces Cytokine Signaling Events in Primary PBMC-Derived Human T Cells Expressing G21R-1 or G21R-2

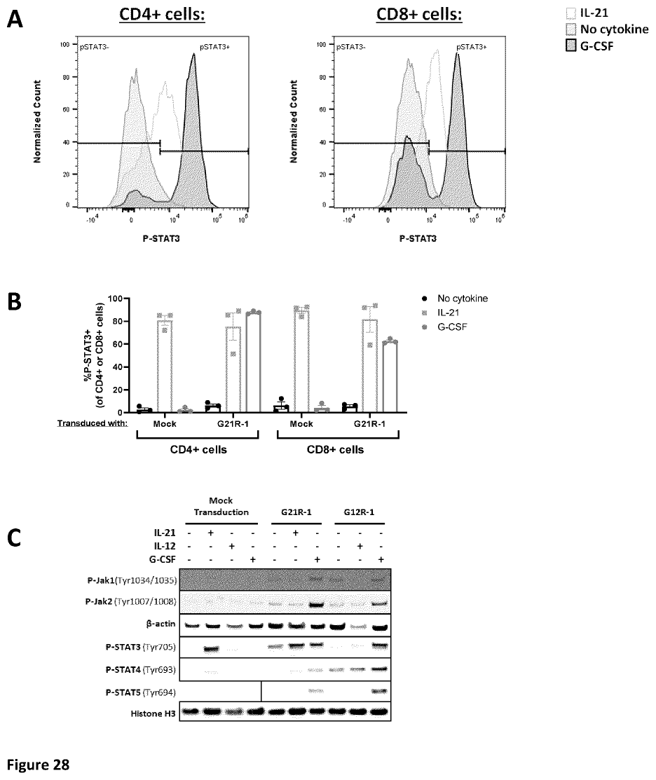

To determine whether the G21R-1 and G21R-2 constructs are capable of inducing cytokine signaling events in primary cells, primary PBMC-derived human T cells were transduced with lentiviral vectors encoding G21R-1 or G21R-2 chimeric cytokine receptors. Cells were subjected to intracellular staining with phospho-STAT3 (p-STAT3) specific antibody and assessed by flow cytometry to determine the extent of STAT3 phosphorylation, a measure of STAT3 activation (FIG. 27). Upon stimulation with G-CSF (100 ng/ml), the number of cells expressing phosphorylated STAT3 increased among the subset of G-CSFR positive cells transduced with either G21R-1 or G21R-2. In contrast, the G-CSFR negative (i.e. non-expressing) cells did not exhibit an increase in phosphorylated STAT3 upon stimulation with G-CSF but did upon stimulation with IL-21.

These results demonstrate that the G21R-1 and G21R-2 chimeric cytokine receptors are capable of activating IL-21-related cytokine signaling events upon stimulation with G-CSF in primary human T cells.

### Example 15: G-CSF Induces Intracellular Signaling Events in Primary Murine T Cells Expressing G21R-1 or G-12R-1

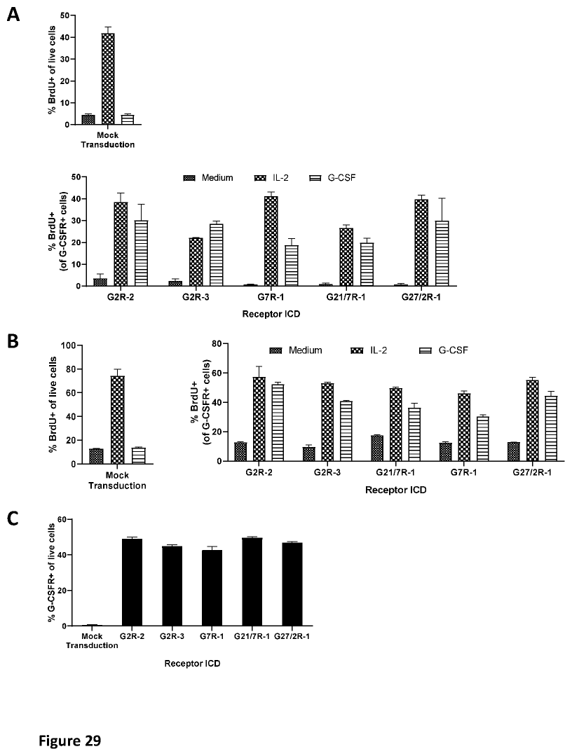

To determine if the chimeric cytokine receptor G21R-1 was capable of activating cytokine signaling events, primary murine T cells were transduced with a retroviral vector encoding G21R-1 and assessed by flow cytometry to detect phosphorylated STAT3 upon stimulation with G-CSF. Live cells were gated on CD8 or CD4, and the percentage of cells staining positive for phospho-STAT3 after stimulation with no cytokine, IL-21 (1 ng/ml) or G-CSF (100 ng/ml) was determined for the CD8 and CD4 cell populations. Upon stimulation with G-CSF, cells expressing G21R-1 (but not non-transduced cells) showed increased amounts of phosphorylated STAT3 (FIGS. 28A and 28B).

Western blots were performed to assess intracellular cytokine signaling in cells expressing G21R-1 or G12R-1 upon stimulation with G-CSF. As expected, cells expressing G21R-1 and stimulated with G-CSF showed increased phosphorylation of STAT3, with minor increases in phospho-STAT4 and phospho-STAT5 (FIG. 28C). Also as expected, in cells expressing G12R-1, strong phosphorylation of STAT4 was seen in response to G-CSF. G-CSF did not induce any signaling events in mock transduced cells. (Note that in the G12R-1 group, the positive control (hIL-12 10 ng/ml) did not appear to induce any signaling events; this may be due to poor binding of human IL-12 to the murine IL-12R.)

The results show that G21R-1 and G12R-1 are capable of inducing cytokine signaling events in primary murine T cells upon stimulation with G-CSF.

### Example 16: G-CSF Induces Proliferation and Intracellular Signaling Events in Primary Murine T Cells Expressing G2R-2, G2R-3, G7R-1, G21/7R-1, G27/2R-1, G21/2R-1, G12/2R-1 or G21/12/2R-1

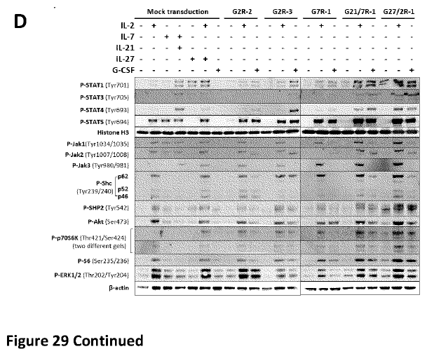

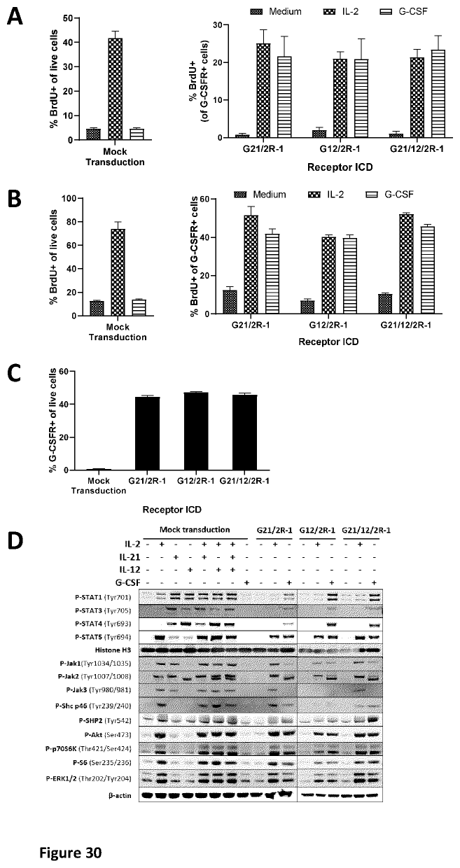

To assess cytokine signaling events and cell proliferation mediated by chimeric cytokine receptors, primary murine T cells were transduced with retroviral vectors encoding G2R-2, G2R-3, G7R-1, G21/7R-1, G27/2R-1, G21/2R-1, G12/2R-1 or G21/12/2R-1. BrdU incorporation assays were performed to assess cell cycle progression upon stimulation with G-CSF. Cells were harvested, washed, and re-plated in IL-2 (300 IU/ml), wildtype G-CSF (100 ng/ml) or no cytokine. G-CSF-induced cell cycle progression was seen in primary murine T cells expressing G2R-2, G2R-3, G7R-1, G21/7R-1 or G27/2R-1 (FIGS. 29A, 29B) or G21/2R-1, G12/2R-1 or G21/12/2R-1 (FIGS. 30A, 30B). Expression of the G-CSFR ECD was also detectable by flow cytometry (FIGS. 29C, 30C).

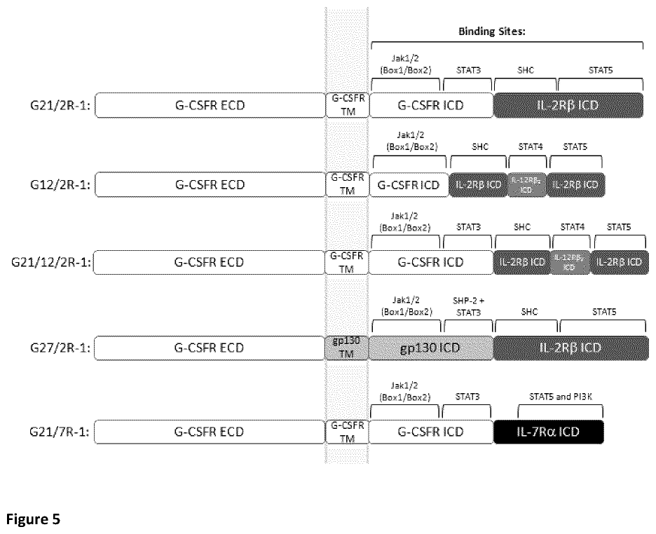

By Western blot, multiple cytokine signaling events were observed in response to G-CSF (100 ng/ml) in cells expressing the indicated chimeric cytokine receptors, but not in mock transduced cells (FIGS. 29D, 30D). In general, the observed cytokine signaling events were as expected based on the signaling domains that were incorporated into the various ICD designs (FIGS. 4 and 5). As one example, the G7R-1 chimeric receptor induced phosphorylation of STAT5 (FIG. 29D), which is expected due to the incorporation of the STAT5 binding site from IL-7Rα (FIG. 4). As a second example, the G21/2R-1 chimeric receptor induced phosphorylation of STAT3 (FIG. 30D), which is expected due to the incorporation of the STAT3 binding site from G-CSFR (FIG. 5). As a third example, the G12/2R-1 chimeric receptor induced phosphorylation of STAT4, which is expected due to the incorporation of the STAT4 binding site from IL-12Rβ₂ (FIG. 5). Other chimeric cytokine receptors showed other patterns of intracellular signaling events.

The results show that G2R-2, G2R-3, G7R-1, G21/7R-1, G27/2R-1, G21/2R-1, G12/2R-1 and G21/12/2R-1 are capable of inducing cytokine signaling events and proliferation in primary murine T cells upon stimulation with G-CSF. Furthermore, different patterns of intracellular signaling events can be generated by incorporating different signaling domains into the ICD of the chimeric receptor.

### Example 17: Orthogonal G-CSF Induces Expansion, Proliferation, Cytokine-Related Intracellular Signaling and Immunophenotype in Primary Human T Cells Expressing G12/2R-1 with Orthogonal ECD

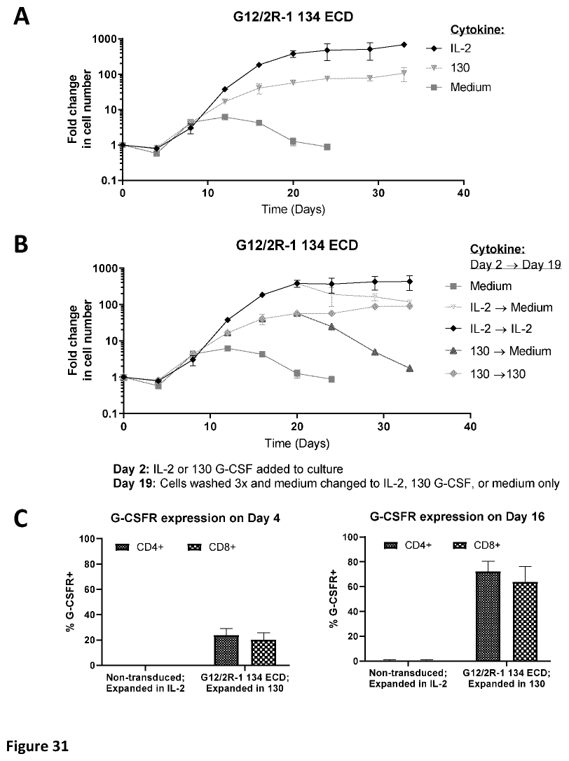

To determine if the chimeric cytokine receptor G12/2R-1 with 134 ECD was capable of inducing proliferation and expansion in response to stimulation with the orthogonal ligand 130 G-CSF, primary PBMC-derived human T cells were transduced with a lentiviral vector encoding G12/2R-1 134 ECD. T-cell growth assays were performed to assess the fold expansion of cells when cultured with IL-2 (300 IU/ml), 130 G-CSF (100 ng/ml) or no cytokine. Live cells were counted every 4-5 days. Primary human T cells expressing G12/2R-1 134 ECD expanded in culture in response to IL-2 or 130 G-CSF (FIG. 31A) but showed limited, transient expansion in medium alone.

On day 19 of this experiment, T cells that had been expanded in 130 G-CSF or IL-2 were washed three times are re-plated in IL-2, 130 G-CSF or medium alone. In medium alone, T cells showed reduced viability and a decline in number (FIG. 31). In contrast, T cells re-plated in IL-2 or G-CSF 130 showed continued viability and stable numbers.

Expression of the G12/2R-1 134 ECD, detected by flow cytometry with an antibody against the G-CSF receptor, increased between Day 4 and Day 16 on both CD4+ and CD8+ T cells expanded by stimulation with 130 G-CSF (FIG. 31C). BrdU incorporation assays were performed to assess cell cycle progression upon stimulation with 130 G-CSF.

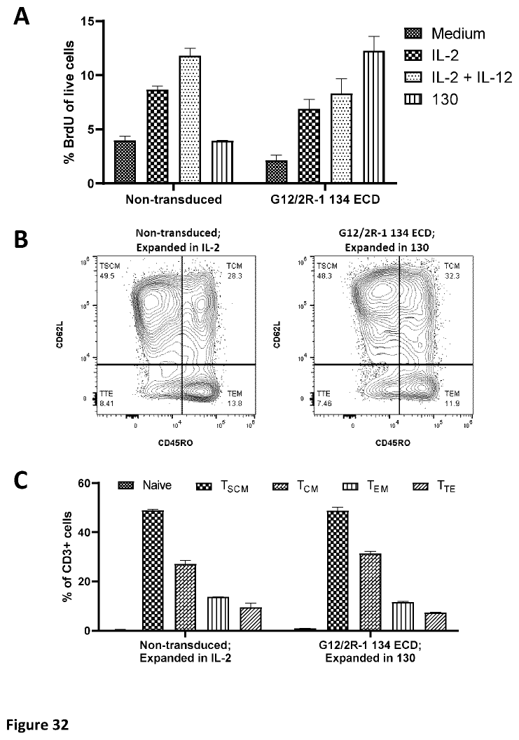

To assess cell cycle progression by BrdU assay, cells were harvested from the expansion assay, washed, and re-plated in IL-2 (300 IU/ml), IL-2 and IL-12 (10 ng/mL), 130 G-CSF (300 ng/ml) or no cytokine. Primary human T cells expressing G12/2R-1 134 ECD demonstrated cell cycle progression in response to 130 G-CSF, IL-2 or IL-2+IL-12, whereas non-transduced cells responded only to IL-2 or IL-2+IL-12 (FIG. 32A).

After a 16 day culture period, immunophenotyping was performed by flow cytometry with antibodies to CD62L and CD45RO to compare T cells expressing G12/2R-1 134 ECD expanded in 130 G-CSF to non-transduced cells expanded in IL-2. The two T cell populations showed similar proportions of stem cell-like memory (T_{SCM}), central memory (T_{CM}), effector memory (T_{EM}), and terminally differentiated (T_{T}) phenotypes (FIG. 32B, C).

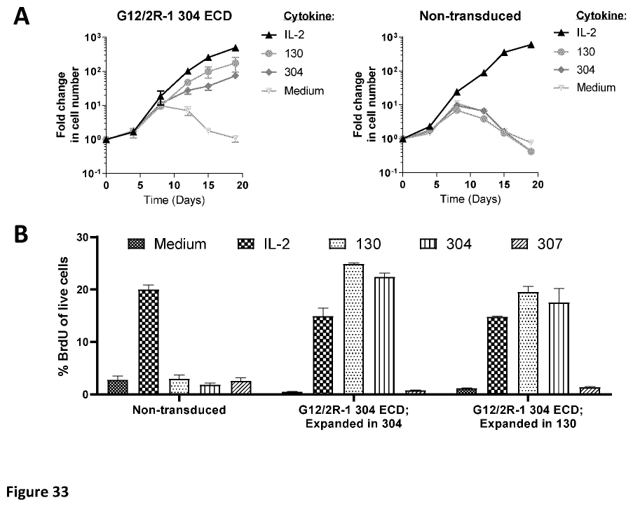

Similar experiments were performed with the chimeric cytokine receptor G12/2R-1 with 304 ECD (as opposed to 134 ECD). Primary PBMC-derived human T cells were transduced with a lentiviral vector encoding G12/2R-1 304 ECD. T-cell growth assays were performed to assess the fold expansion of cells when cultured with IL-2 (300 IU/ml), 130 G-CSF (100 ng/ml), 304 G-CSF (100 ng/mL) or medium alone. Live cells were counted every 4-5 days. T cells expressing G12/2R-1 with 304 ECD could expand in the presence of IL-2, 130 G-CSF or 304 G-CSF, but not in medium alone, while non-transduced cells could expand only in response to IL-2 (FIG. 33A).

To assess cell cycle progression by BrdU assay, T cells expressing G12/2R-1 304 ECD, previously expanded in either 130 G-CSF or 304 G-CSF, were harvested from the expansion assay, washed, and re-plated in IL-2 (300 IU/ml), 130 G-CSF (100 ng/ml), 304 G-CSF (100 ng/mL), 307 G-CSF (100 ng/mL), or medium alone. T cells expressing G12/2R-1 304 ECD demonstrated cell cycle progression in response to 130 or 304 G-CSF, but not in response to 307 G-CSF or medium alone (FIG. 33B).

The results show that G12/2R-1 134 ECD is capable of inducing cell cycle progression and expansion of primary human CD4+ and CD8+ T cells upon stimulation with 130 G-CSF. The T cell memory phenotype of cells expressing G12/2R-1 134 ECD and expanded with 130 G-CSF is similar to non-transduced cells expanded with IL-2. In addition, G12/2R-1 304 ECD is capable of inducing selective cell cycle progression and expansion of T cells upon stimulation with 130 or 304 G-CSF, but not in response to 307 G-CSF.

### Example 18: Orthogonal G-CSF Induces Distinct Intracellular Signaling Events in Primary Human T Cells Expressing G2R-3 or G12/2R-1 with Orthogonal ECD

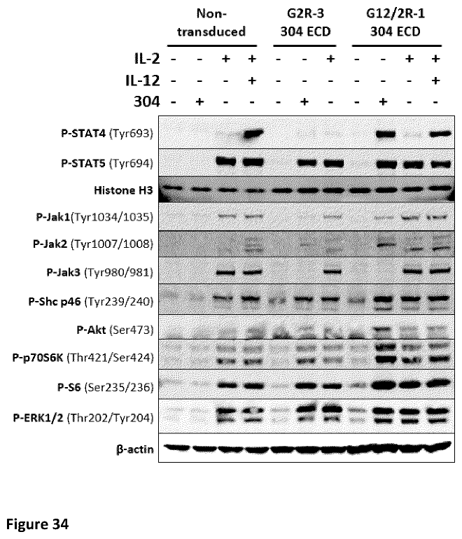

To assess intracellular signaling events, primary PBMC-derived human T cells were transduced with lentiviral vectors encoding G2R-3 304 ECD or G12/2R-1 304 ECD. Western blots were performed to assess intracellular cytokine signaling in cells expressing G2R-3 304 ECD or G12/2R-1 304 ECD, or non-transduced cells, upon stimulation with 304 G-CSF (100 ng/mL), IL-2 (300 IU/mL), IL-2 and IL-12 (10 ng/mL) or medium alone. In both transduced and non-transduced T cells, strong phosphorylation of STAT5 was detected in response to stimulation with either IL-2+IL-12, or IL-2 alone (FIG. 34). Strong phosphorylation of STAT4 was detected in response to stimulation with both IL-2+IL-12, but only weak phosphorylation of STAT4 was detected in response to stimulation with IL-2 alone. In cells expressing G2R-3 304 ECD, weak phosphorylation of STAT4 and strong phosphorylation of STAT5 was detected in response to stimulation with 304 G-CSF, similar to the pattern seen in response to IL-2 alone. In cells expressing G12/2R-1 304 ECD, strong phosphorylation of STAT4 and STAT5 was detected in response to stimulation with 304 G-CSF, similar to the pattern seen in response to IL-2+IL-12. Non-transduced T cells showed no response to 304 G-CSF.

The results show that G12/2R-1 with 304 ECD is capable of inducing cytokine signaling events, including strong phosphorylation of STAT4 and STAT5, in response to stimulation with 304 G-CSF. A different pattern of signaling events is seen in cells expressing G2R-3 304 ECD after stimulation with 304 G-CSF, including strong phosphorylation of STAT5 but not STAT4.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 3
Signal Peptide.
The signal peptide consists of one of the following:

| | | | SEQ |
|---|---|---|---|
| | | | ID |
| | | | NO. |
| Domain | Uniprot ID | Polypeptide Sequence | |
| G-CSFR (Signal | Q99062 | MARLGNCSLTWAALIILLLPGSLE | 1 |
| peptide) | | | |
| GM-CSFR-alpha | P15509 | MLLLVTSLLLCELPHPAFLLIP | 2 |
| (Signal peptide) | | | |
| | Genbank | | |
| Domain | Accession ID | Nucleic Acid Sequence | |
| G-CSFR (Signal | NP_000751 | ATGGCAAGGCTGGGAAACTGCAGCCTGACTTGGGCT | 3 |
| peptide, native | | GCCCTGATCATCCTGCTGCTCCCCGGAAGTCTGGAG | |
| sequence) | | | |
| GM-CSFR-alpha | NP_034100 | ATGCTGCTGCTAGTGACCTCCCTGCTGCTCTGTGAG | 4 |
| (Signal peptide, | | CTGCCTCACCCGGCGTTCCTGCTGATTCCT | |
| native sequence) | | | |

TABLE 4
Wild-type G-CSFR extracellular domain (ECD):
The G-CSFR ECD consists of one of the following:

| | | | SEQ |
|---|---|---|---|
| | | | ID |
| | | | NO. |
| Domain | Uniprot ID | Polypeptide Sequence | |
| G-CSFR (ECD) | Q99062 | ECGHISVSAPIVHLGDPITASCIIKQNCSHLDPEPQILWRLGAELQPGGR | 5 |
| | | QQRLSDGTQESIITLPHLNHTQAFLSCCLNWGNSLQILDQVELRAGYP | |
| | | PAIPHNLSCLMNLTTSSLICQWEPGPETHLPTSFTLKSFKSRGNCQTQG | |
| | | DSILDCVPKDGQSHCCIPRKHLLLYQNMGIWVQAENALGTSMSPQLC | |
| | | LDPMDVVKLEPPMLRTMDPSPEAAPPQAGCLQLCWEPWQPGLHIN | |
| | | QKCELRHKPQRGEASWALVGPLPLEALQYELCGLLPATAYTLQIRCIR | |
| | | WPLPGHWSDWSPSLELRTTERAPTVRLDTWWRQRQLDPRTVQLFW | |
| | | KPVPLEEDSGRIQGYVVSWRPSGQAGAILPLCNTTELSCTFHLPSEAQE | |
| | | VALVAYNSAGTSRPTPVVFSESRGPALTRLHAMARDPHSLWVGWEP | |
| | | PNPWPQGYVIEWGLGPPSASNSNKTWRMEQNGRATGFLLKENIRPF | |
| | | QLYEIIVTPLYQDTMGPSQHVYAYSQEMAPSHAPELHLKHIGKTWAQ | |
| | | LEWVPEPPELGKSPLTHYTIFWTNAQNQSFSAILNASSRGFVLHGLEP | |
| | | ASLYHIHLMAASQAGATNSTVLTLMTLTPEGSELH | |
| | Genbank | | |
| Domain | Accession ID | Nucleic Acid Sequence | |
| G-CSFR (ECD, | NP_000751 | GAGTGCGGGCACATCAGTGTCTCAGCCCCCATCGTCCACCTGGGGG | 6 |
| native sequence) | | ATCCCATCACAGCCTCCTGCATCATCAAGCAGAACTGCAGCCATCTG | |
| | | GACCCGGAGCCACAGATTCTGTGGAGACTGGGAGCAGAGCTTCAG | |
| | | CCCGGGGGCAGGCAGCAGCGTCTGTCTGATGGGACCCAGGAATCT | |
| | | ATCATCACCCTGCCCCACCTCAACCACACTCAGGCCTTTCTCTCCTGC | |
| | | TGCCTGAACTGGGGCAACAGCCTGCAGATCCTGGACCAGGTTGAG | |
| | | CTGCGCGCAGGCTACCCTCCAGCCATACCCCACAACCTCTCCTGCCT | |
| | | CATGAACCTCACAACCAGCAGCCTCATCTGCCAGTGGGAGCCAGGA | |
| | | CCTGAGACCCACCTACCCACCAGCTTCACTCTGAAGAGTTTCAAGAG | |
| | | CCGGGGCAACTGTCAGACCCAAGGGGACTCCATCCTGGACTGCGT | |
| | | GCCCAAGGACGGGCAGAGCCACTGCTGCATCCCACGCAAACACCT | |
| | | GCTGTTGTACCAGAATATGGGCATCTGGGTGCAGGCAGAGAATGC | |
| | | GCTGGGGACCAGCATGTCCCCACAACTGTGTCTTGATCCCATGGAT | |
| | | GTTGTGAAACTGGAGCCCCCCATGCTGCGGACCATGGACCCCAGCC | |
| | | CTGAAGCGGCCCCTCCCCAGGCAGGCTGCCTACAGCTGTGCTGGGA | |
| | | GCCATGGCAGCCAGGCCTGCACATAAATCAGAAGTGTGAGCTGCG | |
| | | CCACAAGCCGCAGCGTGGAGAAGCCAGCTGGGCACTGGTGGGCCC | |
| | | CCTCCCCTTGGAGGCCCTTCAGTATGAGCTCTGCGGGCTCCTCCCAG | |
| | | CCACGGCCTACACCCTGCAGATACGCTGCATCCGCTGGCCCCTGCCT | |
| | | GGCCACTGGAGCGACTGGAGCCCCAGCCTGGAGCTGAGAACTACC | |
| | | GAACGGGCCCCCACTGTCAGACTGGACACATGGTGGCGGCAGAGG | |
| | | CAGCTGGACCCCAGGACAGTGCAGCTGTTCTGGAAGCCAGTGCCCC | |
| | | TGGAGGAAGACAGCGGACGGATCCAAGGTTATGTGGTTTCTTGGA | |
| | | GACCCTCAGGCCAGGCTGGGGCCATCCTGCCCCTCTGCAACACCAC | |
| | | AGAGCTCAGCTGCACCTTCCACCTGCCTTCAGAAGCCCAGGAGGTG | |
| | | GCCCTTGTGGCCTATAACTCAGCCGGGACCTCTCGTCCCACTCCGGT | |
| | | GGTCTTCTCAGAAAGCAGAGGCCCAGCTCTGACCAGACTCCATGCC | |
| | | ATGGCCCGAGACCCTCACAGCCTCTGGGTAGGCTGGGAGCCCCCCA | |
| | | ATCCATGGCCTCAGGGCTATGTGATTGAGTGGGGCCTGGGCCCCCC | |
| | | CAGCGCGAGCAATAGCAACAAGACCTGGAGGATGGAACAGAATG | |
| | | GGAGAGCCACGGGGTTTCTGCTGAAGGAGAACATCAGGCCCTTTC | |
| | | AGCTCTATGAGATCATCGTGACTCCCTTGTACCAGGACACCATGGG | |
| | | ACCCTCCCAGCATGTCTATGCCTACTCTCAAGAAATGGCTCCCTCCC | |
| | | ATGCCCCAGAGCTGCATCTAAAGCACATTGGCAAGACCTGGGCACA | |
| | | GCTGGAGTGGGTGCCTGAGCCCCCTGAGCTGGGGAAGAGCCCCCT | |
| | | TACCCACTACACCATCTTCTGGACCAACGCTCAGAACCAGTCCTTCT | |
| | | CCGCCATCCTGAATGCCTCCTCCCGTGGCTTTGTCCTCCATGGCCTG | |
| | | GAGCCCGCCAGTCTGTATCACATCCACCTCATGGCTGCCAGCCAGG | |
| | | CTGGGGCCACCAACAGTACAGTCCTCACCCTGATGACCTTGACCCC | |
| | | AGAGGGGTCGGAGCTACAC | |
| G-CSFR (ECD, 4 | | GAGTGCGGGCACATCAGTGTCTCAGCCCCCATCGTCCACCTGGGGG | 7 |
| codons altered) | | ATCCCATCACAGCCTCCTGCATCATCAAGCAGAACTGCAGCCATCTG | |
| | | GACCCGGAGCCACAGATTCTGTGGAGACTGGGAGCAGAGCTTCAG | |
| | | CCCGGGGGCAGGCAGCAGCGTCTGTCTGATGGGACCCAGGAATCT | |
| | | ATCATCACCCTGCCCCACCTCAACCACACTCAGGCCTTTCTCTCCTGC | |
| | | TGCCTGAACTGGGGCAACAGCCTGCAGATCCTGGACCAGGTTGAG | |
| | | CTGCGCGCAGGCTACCCTCCAGCCATACCCCACAACCTCTCCTGCCT | |
| | | CATGAACCTCACAACCAGCAGCCTCATCTGCCAGTGGGAGCCAGGA | |
| | | CCTGAGACCCACCTACCCACCAGCTTCACTCTGAAGAGTTTCAAGAG | |
| | | CCGGGGCAACTGTCAGACCCAAGGGGACTCCATCCTGGACTGCGT | |
| | | GCCCAAGGACGGGCAGAGCCACTGCTGCATCCCACGCAAACACCT | |
| | | GCTGTTGTACCAGAATATGGGCATCTGGGTGCAGGCAGAGAATGC | |
| | | GCTGGGGACCAGCATGTCCCCACAACTGTGTCTTGATCCCATGGAT | |
| | | GTTGTGAAACTGGAGCCCCCCATGCTGCGGACCATGGACCCCAGCC | |
| | | CTGAAGCGGCCCCTCCCCAGGCAGGCTGCCTACAGCTGTGCTGGGA | |
| | | GCCATGGCAGCCAGGCCTGCACATAAATCAGAAGTGTGAGCTGCG | |
| | | CCACAAGCCGCAGCGTGGAGAAGCCAGCTGGGCACTGGTGGGCCC | |
| | | CCTCCCCTTGGAGGCCCTTCAGTATGAGCTCTGCGGGCTCCTCCCAG | |
| | | CCACGGCCTACACCCTGCAGATACGCTGCATCCGCTGGCCCCTGCCT | |
| | | GGCCACTGGAGCGACTGGAGCCCCAGCCTGGAGCTGAGAACTACC | |
| | | GAACGGGCCCCCACTGTCAGACTGGACACATGGTGGCGGCAGAGG | |
| | | CAGCTGGACCCCAGGACAGTGCAGCTGTTCTGGAAGCCAGTGCCCC | |
| | | TGGAGGAAGACAGCGGACGCATCCAAGGTTATGTGGTTTCTTGGA | |
| | | GACCCTCAGGCCAGGCTGGGGCCATCCTGCCCCTCTGCAACACCAC | |
| | | AGAGCTCAGCTGCACCTTCCACCTGCCTTCAGAAGCCCAGGAGGTG | |
| | | GCCCTTGTGGCCTATAACTCAGCCGGGACCTCTCGCCCCACCCCGGT | |
| | | GGTCTTCTCAGAAAGCAGAGGCCCAGCTCTGACCAGACTCCATGCC | |
| | | ATGGCCCGAGACCCTCACAGCCTCTGGGTAGGCTGGGAGCCCCCCA | |
| | | ATCCATGGCCTCAGGGCTATGTGATTGAGTGGGGCCTGGGCCCCCC | |
| | | CAGCGCGAGCAATAGCAACAAGACCTGGAGGATGGAACAGAATG | |
| | | GGAGAGCCACGGGGTTTCTGCTGAAGGAGAACATCAGGCCCTTTC | |
| | | AGCTCTATGAGATCATCGTGACTCCCTTGTACCAGGACACCATGGG | |
| | | ACCCTCCCAGCATGTCTATGCCTACTCTCAAGAAATGGCTCCCTCCC | |
| | | ATGCCCCAGAGCTGCATCTAAAGCACATTGGCAAGACCTGGGCACA | |
| | | GCTGGAGTGGGTGCCTGAGCCCCCTGAGCTGGGGAAGAGCCCCCT | |
| | | TACCCACTACACCATCTTCTGGACCAACGCTCAGAACCAGTCCTTCT | |
| | | CCGCCATCCTGAATGCATCCTCCCGTGGCTTTGTCCTCCATGGCCTG | |
| | | GAGCCCGCCAGTCTGTATCACATCCACCTCATGGCTGCCAGCCAGG | |
| | | CTGGGGCCACCAACAGTACAGTCCTCACCCTGATGACCTTGACCCC | |
| | | AGAGGGGTCGGAGCTACAC | |

TABLE 5
Transmembrane domain (TM). The TM consists of one of the following:

| | | | SEQ |
|---|---|---|---|
| | | | ID |
| | | | NO. |
| Domain | Uniprot ID | Polypeptide Sequence | |
| G-CSFR (TM) | Q99062 | IILGLFGLLLLLTCLCGTAWLCC | 8 |
| gp130 (TM) | P40189 | AIVVPVCLAFLLTTLLGVLFCF | 9 |
| IL-2Rb (TM) | P14784 | IPWLGHLLVGLSGAFGFIILVYLLI | 10 |
| IL-2RG (TM) | P31785 | VVISVGSMGLIISLLCVYFWL | 11 |
| | Genbank | | |
| Domain | Accession ID | Nucleic Acid Sequence | |
| G-CSFR (TM) | NP_000751 | ATCATCCTGGGCCTGTTCGGCCTCCTGCTGTTGCTCAC | 12 |
| | | CTGCCTCTGTGGAACTGCCTGGCTCTGTTGC | |
| gp130 (TM) | NM_002184 | GCCATAGTCGTGCCTGTTTGCTTAGCATTCCTATTGAC | 13 |
| | | AACTCTTCTGGGAGTGCTGTTCTGCTTT | |
| IL-2Rb (TM) | NM_000878 | ATTCCGTGGCTCGGCCACCTCCTCGTGGGTCTCAGCGG | 14 |
| | | GGCTTTTGGCTTCATCATCTTAGTGTACTTGCTGATC | |
| IL-2RG (TM) | NP_000197 | GTGGTTATCTCTGTTGGCTCCATGGGATTGATTATCAG | 15 |
| | | CCTTCTCTGTGTGTATTTCTGGCTG | |

TABLE 6
Intracellular domain (ICD). The ICD consists of one of the following:

| | | | SEQ |
|---|---|---|---|
| | | | ID |
| | | | NO. |
| Domain | Uniprot ID | Polypeptide Sequence | |
| IL-2Rb | P14784 | NCRNTGPWLKKVLKCNTPDPSKFFSQLSSEHGGDVQKWLSSPFPSSSF | 16 |
| (ICD, G2R-1) | | SPGGLAPEISPLEVLERDKVTQLLLQQDKVPEPASLSSNHSLTSCFTNQ | |
| | | GYFFFHLPDALEIEACQVYFTYDPYSEEDPDEGVAGAPTGSSPQPLQPL | |
| | | SGEDDAYCTFPSRDDLLLFSPSLLGGPSPPSTAPGGSGAGEERMPPSLQ | |
| | | ERVPRDWDPQPLGPPTPGVPDLVDFQPPPELVLREAGEEVPDAGPRE | |
| | | GVSFPWSRPPGQGEFRALNARLPLNTDAYLSLQELQGQDPTHLV* | |
| IL-2RG | P31785 | ERTMPRIPTLKNLEDLVTEYHGNFSAWSGVSKGLAESLQPDYSERLCLV | 17 |
| (ICD, G2R-1) | | SEIPPKGGALGEGPGASPCNQHSPYWAPPCYTLKPET* | |
| gp130 | P40189 | NKRDLIKKHIWPNVPDPSKSHIAQWSPHTPPRHNFNSKDQMYSDGN | 18 |
| (ICD, G2R-2) | | FTDVSVVEIEANDKKPFPEDLKSLDLFKKEKINTEGHSSGIGGSSCMSSS | |
| | | RPSIS | |
| IL-2Rb | P14784 | ASLSSNHSLTSCFTNQGYFFFHLPDALEIEACQVYFTYDPYSEEDPDEGV | 19 |
| (ICD, G2R-2) | | AGAPTGSSPQPLQPLSGEDDAYCTFPSRDDLLLFSPSLLGGPSPPSTAP | |
| | | GGSGAGEERMPPSLQERVPRDWDPQPLGPPTPGVPDLVDFQPPPEL | |
| | | VLREAGEEVPDAGPREGVSFPWSRPPGQGEFRALNARLPLNTDAYLSL | |
| | | QELQGQDPTHLV* | |
| G-CSFR | Q99062 | SPNRKNPLWPSVPDPAHSSLGSWVPTIMEEDAFQLPGLGTPPITKLTV | 20 |
| (ICD, G2R-3) | | LEEDEKKPVPWESH | |
| IL-2Rb | P14784 | SSNHSLTSCFTNQGYFFFHLPDALEIEACQVYFTYDPYSEEDPDEGVAG | 21 |
| (ICD, G2R-3) | | APTGSSPQPLQPLSGEDDAYCTFPSRDDLLLFSPSLLGGPSPPSTAPGGS | |
| | | GAGEERMPPSLQERVPRDWDPQPLGPPTPGVPDLVDFQPPPELVLRE | |
| | | AGEEVPDAGPREGVSFPWSRPPGQGEFRALNARLPLNTDAYLSLQEL | |
| | | QGQDPTHLV* | |
| G-CSFR | Q99062 | SPNRKNPLWPSVPDPAHSSLGSWVPTIMEEDAFQLPGLGTPPITKLTV | 22 |
| (ICD, G12R-1) | | LEEDEKKPVPWESHNSSET | |
| IL-12Rb2 | Q99665 | AGDLPTHDGYLPSNIDDLPSHEAPLADSLEELEPQHISLSVFPSSSLHPLT | 23 |
| (ICD, G12R-1) | | FSCGDKLTLDQLKMRCDSLML* | |
| G-CSFR | Q99062 | SPNRKNPLWPSVPDPAHSSLGSWVPTIMEEDAFQLPGLGTPPITKLTV | 24 |
| (ICD, G21R-1) | | LEEDEKKPVPWESHNSSET | |
| IL-21R | Q9HBE5 | SPGDEGPPRSYLRQWVVIPPPLSSPGPQAS* | 25 |
| (ICD, G21R-1) | | | |
| G-CSFR | Q99062 | SPNRKNPLWPSVPDPAHSSLGSWVPTIMEEDAFQLPGLGTPPITKLTV | 26 |
| (ICD, G21R-2) | | LEEDEKKPVPWESHN | |
| IL-21R | Q9HBE5 | QNSGGSAYSEERDRPYGLVSIDTVTVLDAEGPCTWPCSCEDDGYPALD | 27 |
| (ICD, G21R-2) | | LDAGLEPSPGLEDPLLDAGTTVLSCGCVSAGSPGLGGPLGSLLDRLKPP | |
| | | LADGEDWAGGLPWGGRSPGGVSESEAGSPLAGLDMDTFDSGFVGS | |
| | | DCSSPVECDFTSPGDEGPPRSYLRQWVVIPPPLSSPGPQAS* | |
| G-CSFR | Q99062 | SPNRKNPLWPSVPDPAHSSLGSWVPTIMEEDAFQLPGLGTPPITKLTV | 28 |
| (ICD, G21/2R-1) | | LEEDEKKPVPWESHNSSETCGLPTLVQTYVLQGDPRAVSTQPQSQ | |
| IL-2Rb | P14784 | SSNHSLTSCFTNQGYFFFHLPDALEIEACQVYFTYDPYSEEDPDEGVAG | 29 |
| (ICD, G21/2R-1) | | APTGSSPQPLQPLSGEDDAYCTFPSRDDLLLFSPSLLGGPSPPSTAPGGS | |
| | | GAGEERMPPSLQERVPRDWDPQPLGPPTPGVPDLVDFQPPPELVLRE | |
| | | AGEEVPDAGPREGVSFPWSRPPGQGEFRALNARLPLNTDAYLSLQEL | |
| | | QGQDPTHLV* | |
| G-CSFR | Q99062 | SPNRKNPLWPSVPDPAHSSLGSWVPTIMEEDAFQLPGLGTPPITKLTV | 30 |
| (ICD, G12/2R-1) | | LEEDEKKPVPWESH | |
| IL-2Rb | P14784 | SSNHSLTSCFTNQGYFFFHLPDALEIEACQVYFTYDPYSEEDP | 31 |
| (ICD, G12/2R-1) | | | |
| IL-12Rb2 | Q99665 | AGDLPTHDGYLPSNIDDLPS | 32 |
| (ICD, G12/2R-1) | | | |
| IL-2Rb | P14784 | GGPSPPSTAPGGSGAGEERMPPSLQERVPRDWDPQPLGPPTPGVPD | 33 |
| (ICD, G12/2R-1) | | LVDFQPPPELVLREAGEEVPDAGPREGVSFPWSRPPGQGEFRALNAR | |
| | | LPLNTDAYLSLQELQGQDPTHLV* | |
| G-CSFR | Q99062 | SPNRKNPLWPSVPDPAHSSLGSWVPTIMEEDAFQLPGLGTPPITKLTV | 34 |
| (ICD, G21/12/2R-1) | | LEEDEKKPVPWESHNSSETCGLPTLVQTYVLQGDPRAVSTQPQSQ | |
| IL-2Rb | P14784 | SSNHSLTSCFTNQGYFFFHLPDALEIEACQVYFTYDPYSEEDP | 35 |
| (ICD, G21/12/2R-1) | | | |
| IL-12Rb2 | Q99665 | AGDLPTHDGYLPSNIDDLPS | 36 |
| (ICD, G21/12/2R-1) | | | |
| IL-2Rb | P14784 | GGPSPPSTAPGGSGAGEERMPPSLQERVPRDWDPQPLGPPTPGVPD | 37 |
| (ICD, G21/12/2R-1) | | LVDFQPPPELVLREAGEEVPDAGPREGVSFPWSRPPGQGEFRALNAR | |
| | | LPLNTDAYLSLQELQGQDPTHLV* | |
| gp130 | P40189 | NKRDLIKKHIWPNVPDPSKSHIAQWSPHTPPRHNFNSKDQMYSDGN | 38 |
| (ICD, G27/2R-1) | | FTDVSVVEIEANDKKPFPEDLKSLDLFKKEKINTEGHSSGIGGSSCMSSS | |
| | | RPSISSSDENESSQNTSSTVQYSTVVHSGYRHQVPSVQVFSR | |
| IL-2Rb | P14784 | ASLSSNHSLTSCFTNQGYFFFHLPDALEIEACQVYFTYDPYSEEDPDEGV | 39 |
| (ICD, G27/2R-1) | | AGAPTGSSPQPLQPLSGEDDAYCTFPSRDDLLLFSPSLLGGPSPPSTAP | |
| | | GGSGAGEERMPPSLQERVPRDWDPQPLGPPTPGVPDLVDFQPPPEL | |
| | | VLREAGEEVPDAGPREGVSFPWSRPPGQGEFRALNARLPLNTDAYLSL | |
| | | QELQGQDPTHLV* | |
| G-CSFR | Q99062 | SPNRKNPLWPSVPDPAHSSLGSWVPTIMEEDAFQLPGLGTPPITKLTV | 40 |
| (ICD, G7R-1) | | LEEDEKKPVPWESH | |
| IL-7Ra | P16871 | SGKNGPHVYQDLLLSLGTTNSTLPPPFSLQSGILTLNPVAQGQPILTSLG | 41 |
| (ICD, G7R-1) | | SNQEEAYVTMSSFYQNQ* | |
| G-CSFR | Q99062 | SPNRKNPLWPSVPDPAHSSLGSWVPTIMEEDAFQLPGLGTPPITKLTV | 42 |
| (ICD, G21/7R-1) | | LEEDEKKPVPWESHNSSETCGLPTLVQTYVLQGDPRAVSTQPQSQ | |
| IL7R | P16871 | SSSRSLDCRESGKNGPHVYQDLLLSLGTTNSTLPPPFSLQSGILTLNPVA | 43 |
| (ICD, G21/7R-1) | | QGQPILTSLGSNQEEAYVTMSSFYQNQ* | |
| | Genbank | | |
| Domain | Accession ID | Nucleic Acid Sequence | |
| IL-2RB | NM_000878 | AACTGCAGGAACACCGGGCCATGGCTGAAGAAGGTCCTGAAGTGT | 44 |
| (ICD, G2R-1) | | AACACCCCAGACCCCTCGAAGTTCTTTTCCCAGCTGAGCTCAGAGCA | |
| | | TGGAGGAGACGTCCAGAAGTGGCTCTCTTCGCCCTTCCCCTCATCGT | |
| | | CCTTCAGCCCTGGCGGCCTGGCACCTGAGATCTCGCCACTAGAAGT | |
| | | GCTGGAGAGGGACAAGGTGACGCAGCTGCTCCTGCAGCAGGACAA | |
| | | GGTGCCTGAGCCCGCATCCTTAAGCAGCAACCACTCGCTGACCAGC | |
| | | TGCTTCACCAACCAGGGTTACTTCTTCTTCCACCTCCCGGATGCCTTG | |
| | | GAGATAGAGGCCTGCCAGGTGTACTTTACTTACGACCCCTACTCAG | |
| | | AGGAAGACCCTGATGAGGGTGTGGCCGGGGCACCCACAGGGTCTT | |
| | | CCCCCCAACCCCTGCAGCCTCTGTCAGGGGAGGACGACGCCTACTG | |
| | | CACCTTCCCCTCCAGGGATGACCTGCTGCTCTTCTCCCCCAGTCTCCT | |
| | | CGGTGGCCCCAGCCCCCCAAGCACTGCCCCTGGGGGCAGTGGGGC | |
| | | CGGTGAAGAGAGGATGCCCCCTTCTTTGCAAGAAAGAGTCCCCAGA | |
| | | GACTGGGACCCCCAGCCCCTGGGGCCTCCCACCCCAGGAGTCCCAG | |
| | | ACCTGGTGGATTTTCAGCCACCCCCTGAGCTGGTGCTGCGAGAGGC | |
| | | TGGGGAGGAGGTCCCTGACGCTGGCCCCAGGGAGGGAGTCAGTTT | |
| | | CCCCTGGTCCAGGCCTCCTGGGCAGGGGGAGTTCAGGGCCCTTAAT | |
| | | GCTCGCCTGCCCCTGAACACTGATGCCTACTTGTCCCTCCAAGAACT | |
| | | CCAGGGTCAGGACCCAACTCACTTGGTGTAG | |
| IL-2RG | NP_000197 | GAACGGACGATGCCCCGAATTCCCACCCTGAAGAACCTAGAGGATC | 45 |
| (ICD, G2R-1) | | TTGTTACTGAATACCACGGGAACTTTTCGGCCTGGAGTGGTGTGTCT | |
| | | AAGGGACTGGCTGAGAGTCTGCAGCCAGACTACAGTGAACGACTCT | |
| | | GCCTCGTCAGTGAGATTCCCCCAAAAGGAGGGGCCCTTGGGGAGG | |
| | | GGCCTGGGGCCTCCCCATGCAACCAGCATAGCCCCTACTGGGCCCC | |
| | | CCCATGTTACACCCTAAAGCCTGAAACCTGA | |
| gp130 | NM_002184 | AATAAGCGAGACCTAATTAAAAAACACATCTGGCCTAATGTTCCAG | 46 |
| (ICD, G2R-2) | | ATCCTTCAAAGAGTCATATTGCCCAGTGGTCACCTCACACTCCTCCA | |
| | | AGGCACAATTTTAATTCAAAAGATCAAATGTATTCAGATGGCAATTT | |
| | | CACTGATGTAAGTGTTGTGGAAATAGAAGCAAATGACAAAAAGCCT | |
| | | TTTCCAGAAGATCTGAAATCATTGGACCTGTTCAAAAAGGAAAAAA | |
| | | TTAATACTGAAGGACACAGCAGTGGTATTGGGGGGTCTTCATGTAT | |
| | | GTCATCTTCTAGGCCAAGCATTTCT | |
| IL-2Rb | NM_000878 | GCATCCTTAAGCAGCAACCACTCGCTGACCAGCTGCTTCACCAACCA | 47 |
| (ICD, G2R-2) | | GGGTTACTTCTTCTTCCACCTCCCGGATGCCTTGGAGATAGAGGCCT | |
| | | GCCAGGTGTACTTTACTTACGACCCCTACTCAGAGGAAGACCCTGAT | |
| | | GAGGGTGTGGCCGGGGCACCCACAGGGTCTTCCCCCCAACCCCTGC | |
| | | AGCCTCTGTCAGGGGAGGACGACGCCTACTGCACCTTCCCCTCCAG | |
| | | GGATGACCTGCTGCTCTTCTCCCCCAGTCTCCTCGGTGGCCCCAGCC | |
| | | CCCCAAGCACTGCCCCTGGGGGCAGTGGGGCCGGTGAAGAGAGGA | |
| | | TGCCCCCTTCTTTGCAAGAAAGAGTCCCCAGAGACTGGGACCCCCA | |
| | | GCCCCTGGGGCCTCCCACCCCAGGAGTCCCAGACCTGGTGGATTTT | |
| | | CAGCCACCCCCTGAGCTGGTGCTGCGAGAGGCTGGGGAGGAGGTC | |
| | | CCTGACGCTGGCCCCAGGGAGGGAGTCAGTTTCCCCTGGTCCAGGC | |
| | | CTCCTGGGCAGGGGGAGTTCAGGGCCCTTAATGCTCGCCTGCCCCT | |
| | | GAACACTGATGCCTACTTGTCCCTCCAAGAACTCCAGGGTCAGGAC | |
| | | CCAACTCACTTGGTGTAG | |
| G-CSFR | NP_000751 | AGCCCCAACAGGAAGAATCCCCTCTGGCCAAGTGTCCCAGACCCAG | 48 |
| (ICD, G2R-3) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | AT | |
| IL-2Rb | NM_000878 | AGCAGCAACCACTCGCTGACCAGCTGCTTCACCAACCAGGGTTACTT | 49 |
| (ICD, G2R-3) | | CTTCTTCCACCTCCCGGATGCCTTGGAGATAGAGGCCTGCCAGGTGT | |
| | | ACTTTACTTACGACCCCTACTCAGAGGAAGACCCTGATGAGGGTGT | |
| | | GGCCGGGGCACCCACAGGGTCTTCCCCCCAACCCCTGCAGCCTCTG | |
| | | TCAGGGGAGGACGACGCCTACTGCACCTTCCCCTCCAGGGATGACC | |
| | | TGCTGCTCTTCTCCCCCAGTCTCCTCGGTGGCCCCAGCCCCCCAAGC | |
| | | ACTGCCCCTGGGGGCAGTGGGGCCGGTGAAGAGAGGATGCCCCCT | |
| | | TCTTTGCAAGAAAGAGTCCCCAGAGACTGGGACCCCCAGCCCCTGG | |
| | | GGCCTCCCACCCCAGGAGTCCCAGACCTGGTGGATTTTCAGCCACCC | |
| | | CCTGAGCTGGTGCTGCGAGAGGCTGGGGAGGAGGTCCCTGACGCT | |
| | | GGCCCCAGGGAGGGAGTCAGTTTCCCCTGGTCCAGGCCTCCTGGGC | |
| | | AGGGGGAGTTCAGGGCCCTTAATGCTCGCCTGCCCCTGAACACTGA | |
| | | TGCCTACTTGTCCCTCCAAGAACTCCAGGGTCAGGACCCAACTCACT | |
| | | TGGTGTAG | |
| G-CSFR | NP_000751 | AGCCCCAACAGGAAGAATCCCCTCTGGCCAAGTGTCCCAGACCCAG | 50 |
| (ICD, G12R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAACAGCTCAGAGACC | |
| IL-12Rb2 | NP_001550 | GCAGGTGACCTTCCCACCCATGATGGCTACTTACCCTCCAACATAGA | |
| (ICD, G12R-1) | | TGACCTCCCCTCACATGAGGCACCTCTCGCTGACTCTCTGGAAGAAC | |
| | | TGGAGCCTCAGCACATCTCCCTTTCTGTTTTCCCCTCAAGTTCTCTTC | |
| | | ACCCACTCACCTTCTCCTGTGGTGATAAGCTGACTCTGGATCAGTTA | |
| | | AAGATGAGGTGTGACTCCCTCATGCTCTGA | |
| G-CSFR | NP_000751 | AGCCCCAACAGGAAGAATCCCCTCTGGCCAAGTGTCCCAGACCCAG | 52 |
| (ICD, G21R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAACAGCTCAGAGACC | |
| IL-21R | NP_068570 | AGCCCTGGGGACGAAGGACCCCCCCGGAGCTACCTCCGCCAGTGG | 53 |
| (ICD, G21R-1) | (1codon | GTGGTCATTCCTCCGCCACTTTCGAGCCCTGGACCCCAGGCCAGCTA | |
| | altered) | A | |
| G-CSFR | NP_000751 | AGCCCCAACAGGAAGAATCCCCTCTGGCCAAGTGTCCCAGACCCAG | 54 |
| (ICD, G21R-2) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAAC | |
| IL-21R | NP_068570 | CAGAACTCGGGGGGCTCAGCTTACAGTGAGGAGAGGGATCGGCCA | 55 |
| (ICD, G21R-2) | (1codon | TACGGCCTGGTGTCCATTGACACAGTGACTGTGCTAGATGCAGAGG | |
| | altered) | GGCCATGCACCTGGCCCTGCAGCTGTGAGGATGACGGCTACCCAGC | |
| | | CCTGGACCTGGATGCTGGCCTGGAGCCCAGCCCAGGCCTAGAGGA | |
| | | CCCACTCTTGGATGCAGGGACCACAGTCCTGTCCTGTGGCTGTGTCT | |
| | | CAGCTGGCAGCCCTGGGCTAGGAGGGCCCCTGGGAAGCCTCCTGG | |
| | | ACAGACTAAAGCCACCCCTTGCAGATGGGGAGGACTGGGCTGGGG | |
| | | GACTGCCCTGGGGTGGCCGGTCACCTGGAGGGGTCTCAGAGAGTG | |
| | | AGGCGGGCTCACCCCTGGCCGGCCTGGATATGGACACGTTTGACAG | |
| | | TGGCTTTGTGGGCTCTGACTGCAGCAGCCCTGTGGAGTGTGACTTC | |
| | | ACCAGCCCTGGGGACGAAGGACCCCCCCGGAGCTACCTCCGCCAGT | |
| | | GGGTGGTCATTCCTCCGCCACTTTCGAGCCCTGGACCCCAGGCCAG | |
| | | C | |
| G-CSFR | NP_000751 | AGCCCCAACAGGAAGAATCCCCTCTGGCCAAGTGTCCCAGACCCAG | 56 |
| (ICD, G21/2R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAACAGCTCAGAGACCTGTGGCCTCCCCACTCTGGTCCAGACCTAT | |
| | | GTGCTCCAGGGGGACCCAAGAGCAGTTTCCACCCAGCCCCAATCCC | |
| | | AG | |
| IL-2Rb | NM_000878 | AGCAGCAACCACTCGCTGACCAGCTGCTTCACCAACCAGGGTTACTT | 57 |
| (ICD, G21/2R-1) | | CTTCTTCCACCTCCCGGATGCCTTGGAGATAGAGGCCTGCCAGGTGT | |
| | | ACTTTACTTACGACCCCTACTCAGAGGAAGACCCTGATGAGGGTGT | |
| | | GGCCGGGGCACCCACAGGGTCTTCCCCCCAACCCCTGCAGCCTCTG | |
| | | TCAGGGGAGGACGACGCCTACTGCACCTTCCCCTCCAGGGATGACC | |
| | | TGCTGCTCTTCTCCCCCAGTCTCCTCGGTGGCCCCAGCCCCCCAAGC | |
| | | ACTGCCCCTGGGGGCAGTGGGGCCGGTGAAGAGAGGATGCCCCCT | |
| | | TCTTTGCAAGAAAGAGTCCCCAGAGACTGGGACCCCCAGCCCCTGG | |
| | | GGCCTCCCACCCCAGGAGTCCCAGACCTGGTGGATTTTCAGCCACCC | |
| | | CCTGAGCTGGTGCTGCGAGAGGCTGGGGAGGAGGTCCCTGACGCT | |
| | | GGCCCCAGGGAGGGAGTCAGTTTCCCCTGGTCCAGGCCTCCTGGGC | |
| | | AGGGGGAGTTCAGGGCCCTTAATGCTCGCCTGCCCCTGAACACTGA | |
| | | TGCCTACTTGTCCCTCCAAGAACTCCAGGGTCAGGACCCAACTCACT | |
| | | TGGTGTAG | |
| G-CSFR | NP_000751 | AGCCCCAACAGGAAGAATCCCCTCTGGCCAAGTGTCCCAGACCCAG | 58 |
| (ICD, G12/2R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | AT | |
| IL-2Rb | NM_000878 | AGCAGCAACCACTCGCTGACCAGCTGCTTCACCAACCAGGGTTACTT | 59 |
| (ICD, G12/2R-1) | | CTTCTTCCACCTCCCGGATGCCTTGGAGATAGAGGCCTGCCAGGTGT | |
| | | ACTITACTTACGACCCCTACTCAGAGGAAGACCCT | |
| IL-12Rb2 | NP_001550 | GCAGGTGACCTTCCCACCCATGATGGCTACTTACCCTCCAACATAGA | 60 |
| (ICD, G12/2R-1) | | TGACCTCCCCTCA | |
| IL-2Rb | NM_000878 | GGTGGCCCCAGCCCCCCAAGCACTGCCCCTGGGGGCAGTGGGGCC | 61 |
| (ICD, G12/2R-1) | | GGTGAAGAGAGGATGCCCCCTTCTTTGCAAGAAAGAGTCCCCAGAG | |
| | | ACTGGGACCCCCAGCCCCTGGGGCCTCCCACCCCAGGAGTCCCAGA | |
| | | CCTGGTGGATTTTCAGCCACCCCCTGAGCTGGTGCTGCGAGAGGCT | |
| | | GGGGAGGAGGTCCCTGACGCTGGCCCCAGGGAGGGAGTCAGTTTC | |
| | | CCCTGGTCCAGGCCTCCTGGGCAGGGGGAGTTCAGGGCCCTTAATG | |
| | | CTCGCCTGCCCCTGAACACTGATGCCTACTTGTCCCTCCAAGAACTC | |
| | | CAGGGTCAGGACCCAACTCACTTGGTGTAG | |
| G-CSFR | NP_000751 | AGCCCCAACAGGAAGAATCCCCTCTGGCCAAGTGTCCCAGACCCAG | 62 |
| (ICD, G21/12/2R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAACAGCTCAGAGACCTGTGGCCTCCCCACTCTGGTCCAGACCTAT | |
| | | GTGCTCCAGGGGGACCCAAGAGCAGTTTCCACCCAGCCCCAATCCC | |
| | | AG | |
| IL-2Rb | NM_000878 | AGCAGCAACCACTCGCTGACCAGCTGCTTCACCAACCAGGGTTACTT | 63 |
| (ICD, G21/12/2R-1) | | CTTCTTCCACCTCCCGGATGCCTTGGAGATAGAGGCCTGCCAGGTGT | |
| | | ACTITACTTACGACCCCTACTCAGAGGAAGACCCT | |
| IL-12Rb2 | NP_001550 | GCAGGTGACCTTCCCACCCATGATGGCTACTTACCCTCCAACATAGA | 64 |
| (ICD, G21/12/2R-1) | | TGACCTCCCCTCA | |
| IL-2Rb | NM_000878 | GGTGGCCCCAGCCCCCCAAGCACTGCCCCTGGGGGCAGTGGGGCC | 65 |
| (ICD, G21/12/2R-1) | | GGTGAAGAGAGGATGCCCCCTTCTTTGCAAGAAAGAGTCCCCAGAG | |
| | | ACTGGGACCCCCAGCCCCTGGGGCCTCCCACCCCAGGAGTCCCAGA | |
| | | CCTGGTGGATTTTCAGCCACCCCCTGAGCTGGTGCTGCGAGAGGCT | |
| | | GGGGAGGAGGTCCCTGACGCTGGCCCCAGGGAGGGAGTCAGTTTC | |
| | | CCCTGGTCCAGGCCTCCTGGGCAGGGGGAGTTCAGGGCCCTTAATG | |
| | | CTCGCCTGCCCCTGAACACTGATGCCTACTTGTCCCTCCAAGAACTC | |
| | | CAGGGTCAGGACCCAACTCACTTGGTGTAG | |
| gp130 | NM_002184 | AATAAGCGAGACCTAATTAAAAAACACATCTGGCCTAATGTTCCAG | 66 |
| (ICD, G27/2R-1) | (3 codons | ATCCTTCAAAGAGTCATATTGCCCAGTGGTCACCTCACACTCCTCCA | |
| | altered) | AGGCACAATTTCAATTCAAAGGATCAAATGTATTCAGATGGCAATTT | |
| | | CACTGATGTAAGTGTTGTGGAAATAGAAGCAAATGACAAAAAGCCT | |
| | | TTTCCAGAAGATCTGAAATCATTGGACCTGTTCAAAAAGGAAAAAA | |
| | | TTAATACTGAAGGACACAGCAGTGGTATTGGGGGGTCTTCATGTAT | |
| | | GTCATCTTCTAGGCCAAGCATTTCTAGCAGTGATGAAAATGAATCTT | |
| | | CACAAAACACTTCGAGCACTGTCCAGTATTCTACCGTGGTACACAGT | |
| | | GGCTACAGACACCAAGTTCCGTCAGTCCAAGTCTTCTCAAGA | |
| IL-2Rb | NM_000878 | GCATCCTTAAGCAGCAACCACTCGCTGACCAGCTGCTTCACCAACCA | 67 |
| (ICD, G27/2R-1) | (2 codons | GGGTTACTTCTTCTTCCACCTCCCGGATGCCTTGGAGATAGAGGCCT | |
| | altered) | GCCAGGTGTACTTTACTTACGACCCCTACTCAGAGGAAGACCCTGAT | |
| | | GAGGGTGTGGCCGGGGCACCCACAGGGTCTTCCCCCCAACCCCTGC | |
| | | AGCCTCTGTCAGGGGAGGACGACGCCTACTGCACCTTCCCCTCCAG | |
| | | GGATGACCTGCTGCTCTTCTCCCCCAGTCTCCTCGGTGGCCCCAGCC | |
| | | CCCCAAGCACTGCCCCTGGGGGCAGTGGGGCCGGTGAAGAGAGGA | |
| | | TGCCCCCTTCTTTGCAAGAAAGAGTCCCCAGAGACTGGGACCCCCA | |
| | | GCCCCTGGGGCCTCCCACCCCAGGAGTCCCAGACCTGGTGGATTTT | |
| | | CAGCCACCCCCTGAGCTGGTGCTGCGAGAGGCTGGGGAGGAGGTC | |
| | | CCTGACGCTGGCCCCAGGGAGGGAGTCAGTTTCCCCTGGTCCAGGC | |
| | | CTCCTGGGCAGGGGGAGTTCAGGGCCCTTAATGCTCGCCTGCCCCT | |
| | | GAACACTGATGCCTACTTGTCCCTCCAAGAACTCCAGGGTCAGGAC | |
| | | CCAACTCACTTGGTGTAG | |
| G-CSFR | NP_000751 | AGCCCCAACAGGAAGAATCCCCTCTGGCCAAGTGTCCCAGACCCAG | 68 |
| (ICD, G7R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | AT | |
| IL-7Ra | NP_002176 | AGTGGCAAGAATGGGCCTCATGTGTACCAGGACCTCCTGCTTAGCC | 69 |
| (ICD, G7R-1) | | TTGGGACTACAAACAGCACGCTGCCCCCTCCATTTTCTCTCCAATCTG | |
| | | GAATCCTGACATTGAACCCAGTTGCTCAGGGTCAGCCCATTCTTACT | |
| | | TCCCTGGGATCAAATCAAGAAGAAGCATATGTCACCATGTCCAGCTT | |
| | | CTACCAAAACCAGTGA | |
| G-CSFR | NP_000751 | AGCCCCAACAGGAAGAATCCCCTCTGGCCAAGTGTCCCAGACCCAG | 70 |
| (ICD, G21/7R-1) | | CTCACAGCAGCCTGGGCTCCTGGGTGCCCACAATCATGGAGGAGGA | |
| | | TGCCTTCCAGCTGCCCGGCCTTGGCACGCCACCCATCACCAAGCTCA | |
| | | CAGTGCTGGAGGAGGATGAAAAGAAGCCGGTGCCCTGGGAGTCCC | |
| | | ATAACAGCTCAGAGACCTGTGGCCTCCCCACTCTGGTCCAGACCTAT | |
| | | GTGCTCCAGGGGGACCCAAGAGCAGTTTCCACCCAGCCCCAATCCC | |
| | | AG | |
| IL7R | NP_002176 | TCCTCTTCCAGGTCCCTAGACTGCAGGGAGAGTGGCAAGAATGGGC | 71 |
| (ICD, G21/7R-1) | | CTCATGTGTACCAGGACCTCCTGCTTAGCCTTGGGACTACAAACAGC | |
| | | ACGCTGCCCCCTCCATTTTCTCTCCAATCTGGAATCCTGACATTGAAC | |
| | | CCAGTTGCTCAGGGTCAGCCCATTCTTACTTCCCTGGGATCAAATCA | |
| | | AGAAGAAGCATATGTCACCATGTCCAGCTTCTACCAAAACCAGTGA | |

TABLE 7
G-CSF Sequences

| | | | SEQ |
|---|---|---|---|
| | | | ID |
| | | | NO. |
| Name | Uniprot ID | Polypeptide Sequence | |
| Human G-CSF | A0A0B4U5E3 | TPLGPASSLPQSFLLKCLEQVRKIQGDGAALQEKLCATYKLCHPEELVLL | 72 |
| | | GHSLGIPWAPLSSCPSQALQLAGCLSQLHSGLFLYQGLLQALEGISPEL | |
| | | GPTLDTLQLDVADFATTIWQQMEELGMAPALQPTQGAMPAFASAF | |
| | | QRRAGGVLVASHLOSFLEVSYRVLRHLAQP | |
| Human G-CSF | | MTPLGPASSLPQSFLLKCLEQVRKIQGDGAALQEKLCATYKLCHPEELV | 80 |
| recombinantly | | LLGHSLGIPWAPLSSCPSQALQLAGCLSQLHSGLFLYQGLLQALEGISPE | |
| produced in | | LGPTLDTLQLDVADFATTIWQQMEELGMAPALQPTQGAMPAFASAF | |
| E. coli | | QRRAGGVLVASHLQSFLEVSYRVLRHLAQP | |
| | Genbank | | |
| Name | Accession ID | Nucleic Acid Sequence | |
| Human G-CSF | NM_172220 | AGCCCGGAGC CTGCAGCCCA GCCCCACCCA GACCCATGGC | 73 |
| | | TGGACCTGCC ACCCAGAGCC CCATGAAGCT GATGGCCCTG | |
| | | CAGCTGCTGC TGTGGCACAG TGCACTCTGG ACAGTGCAGG | |
| | | AAGCCACCCC CCTGGGCCCT GCCAGCTCCC TGCCCCAGAG | |
| | | CTTCCTGCTC AAGTGCTTAG AGCAAGTGAG | |
| | | GAAGATCCAG GGCGATGGCG CAGCGCTCCA GGAGAAGCTG | |
| | | GTGAGTGAGG CAGGCTGCTT GAGCCAACTC CATAGCGGCC | |
| | | TTTTCCTCTA CCAGGGGCTC CTGCAGGCCC TGGAAGGGAT | |
| | | CTCCCCCGAG TTGGGTCCCA CCTTGGACAC ACTGCAGCTG | |
| | | GACGTCGCCG ACTTTGCCAC CACCATCTGG CAGCAGATGG | |
| | | AAGAACTGGG AATGGCCCCT GCCCTGCAGC CCACCCAGGG | |
| | | TGCCATGCCG GCCTTCGCCT CTGCTTTCCA GCGCCGGGCA | |
| | | GGAGGGGTCC TGGTTGCCTC CCATCTGCAG AGCTTCCTGG | |
| | | AGGTGTCGTA CCGCGTTCTA CGCCACCTTG CCCAGCCCTG | |
| | | AGCCAAGCCC TCCCCATCCC ATGTATTTAT CTCTATTTAA | |
| | | TATTTATGTC TATTTAAGCC TCATATTTAA AGACAGGGAA | |
| | | GAGCAGAACG GAGCCCCAGG CCTCTGTGTC CTTCCCTGCA | |
| | | TTTCTGAGTT TCATTCTCCT GCCTGTAGCA GTGAGAAAAA | |
| | | GCTCCTGTCC TCCCATCCCC TGGACTGGGA GGTAGATAGG | |
| | | TAAATACCAA GTATTTATTA CTATGACTGC TCCCCAGCCC | |
| | | TGGCTCTGCA ATGGGCACTG GGATGAGCCG CTGTGAGCCC | |
| | | CTGGTCCTGA GGGTCCCCAC CTGGGACCCT TGAGAGTATC | |
| | | AGGTCTCCCA CGTGGGAGAC AAGAAATCCC TGTTTAATAT | |
| | | TTAAACAGCA GTGTTCCCCA TCTGGGTCCT TGCACCCCTC | |
| | | ACTCTGGCCT CAGCCGACTG CACAGCGGCC CCTGCATCCC | |
| | | CTTGGCTGTG AGGCCCCTGG ACAAGCAGAG GTGGCCAGAG | |
| | | CTGGGAGGCA TGGCCCTGGG GTCCCACGAA TTTGCTGGGG | |
| | | AATCTCGTTT TTCTTCTTAA GACTTTTGGG ACATGGTTTG | |
| | | ACTCCCGAAC ATCACCGACG CGTCTCCTGT TTTTCTGGGT | |
| | | GGCCTCGGGA CACCTGCCCT GCCCCCACGA GGGTCAGGAC | |
| | | TGTGACTCTT TTTAGGGCCA GGCAGGTGCC TGGACATTTG | |
| | | CCTTGCTGGA CGGGGACTGG GGATGTGGGA GGGAGCAGAC | |
| | | AGGAGGAATC ATGTCAGGCC TGTGTGTGAA AGGAAGCTCC | |
| | | ACTGTCACCC TCCACCTCTT CACCCCCCAC TCACCAGTGT | |
| | | CCCCTCCACT GTCACATTGT AACTGAACTT CAGGATAATA | |
| | | AAGTGTTTGC CTCCA | |

1. A chimeric receptor, comprising:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor) and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R;
wherein at least a portion of the ICD of the cytokine receptor comprises at least one signaling molecule binding site from an intracellular domain of a cytokine receptor, and, optionally,
the at least one signaling molecule binding site is selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R;
and, optionally, the ICD comprises a Box 1 region and a Box 2 region of a protein selected from the group consisting of G-CSFR and gp130;
and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain.

2. A chimeric receptor, comprising:
an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

3. The chimeric receptor of claim 1, wherein the activated chimeric receptor forms a homodimer, and, optionally,
the activation of the chimeric receptor causes a cellular response selected from the group consisting of proliferation, viability and enhanced activity of a cell expressing the chimeric receptor, and, optionally, the chimeric receptor is activated upon contact with a G-CSF, and, optionally,
the G-CSF is a wild-type G-CSF, and, optionally,
the extracellular domain of the G-CSFR is a wild-type extracellular domain.

4. The chimeric receptor of claim 2, wherein the activated chimeric receptor forms a homodimer, and, optionally,
the activation of the chimeric receptor causes a cellular response selected from the group consisting of proliferation, viability and enhanced activity of a cell expressing the chimeric receptor, and, optionally,
the chimeric receptor is activated upon contact with a G-CSF, and, optionally,
the G-CSF is a wild-type G-CSF, and, optionally,
the extracellular domain of the G-CSFR is a wild-type extracellular domain.

5. The chimeric receptor of claim 1, wherein the chimeric receptor is expressed in a cell, and, optionally, an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

6. The chimeric receptor of claim 2, wherein the chimeric receptor is expressed in a cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

7. The chimeric receptor of claim 3, wherein the chimeric receptor is expressed in a cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

8. The chimeric receptor of claim 4, wherein the chimeric receptor is expressed in a cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

9. The chimeric receptor of claim 1, wherein the ICD comprises:
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17.

10. The chimeric receptor of claim 2, wherein the ICD comprises:
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17.

11. The chimeric receptor of claim 1, wherein the transmembrane domain comprises a sequence set forth by:
(a) SEQ ID NO. 8; or
(b) SEQ ID NO. 9; or
(c) SEQ ID NO. 10; or
(d) SEQ ID NO. 11.

12. The chimeric receptor of claim 2, wherein the transmembrane domain comprises a sequence set forth by:
(a) SEQ ID NO. 8; or
(b) SEQ ID NO. 9; or
(c) SEQ ID NO. 10; or
(d) SEQ ID NO. 11.

13. A nucleic acid encoding a chimeric receptor; wherein the chimeric receptor comprises:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally,
the IL-2R is selected from the group consisting of IL-2Rβ and IL-2Rγc, and, optionally,
the second domain comprises at least a portion of the C-terminal region of IL-2Rβ, IL-7Rα, IL-12Rβ₂ or IL-21R; wherein
at least a portion of the ICD of the cytokine receptor comprises at least one signaling molecule binding site from an intracellular domain of a cytokine receptor, and, optionally,
the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R;
and, optionally, the ICD comprises a Box 1 region and a Box 2 region of a protein selected from the group consisting of G-CSFR and gp130;
and, optionally, the chimeric receptor comprises a third domain comprising at least a portion of a transmembrane domain of a protein selected from the group consisting of: G-CSFR, gp130 (Glycoprotein 130), and IL-2Rβ; and, optionally,
the transmembrane domain is a wild-type transmembrane domain.

14. The nucleic acid of claim 13, wherein the ECD of the G-CSFR is encoded by nucleic acid sequence set forth in SEQ ID NO. 5 or 6.

15. The nucleic acid of claim 13 comprising:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

16. The nucleic acid of claim 14, comprising:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

17. An expression vector comprising the nucleic acid of any one of claims 13-16.

18. The expression vector of claim 17, wherein the vector is selected from the group consisting of: a retroviral vector, a lentiviral vector, an adenoviral vector and a plasmid.

19. A nucleic acid encoding a chimeric receptor; wherein the chimeric receptor comprises:
an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

20. The nucleic acid of claim 19, wherein the ECD of the G-CSFR is encoded by nucleic acid sequence set forth in SEQ ID NO. 5 or 6.

21. The nucleic acid of claim 19, comprising:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

22. The nucleic acid of claim 20, comprising:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

23. An expression vector comprising the nucleic acid of any one of claims 19-22.

24. The expression vector of claim 23, wherein the vector is selected from the group consisting of: a retroviral vector, a lentiviral vector, an adenoviral vector and a plasmid.

25. A cell comprising a nucleic acid encoding a chimeric receptor; wherein the chimeric receptor comprises:
(a) an extracellular domain (ECD) of a G-CSFR (Granulocyte-Colony Stimulating Factor Receptor) operatively linked Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally,
the IL-2R is selected from the group consisting of IL-2Rβ and IL-2Rγc, and, optionally,
the second domain comprises at least a portion of the C-terminal region of IL-2Rβ, IL-7Rα, IL-12Rβ₂ or IL-21R; wherein
at least a portion of the ICD of the cytokine receptor comprises at least one signaling molecule binding site from an intracellular domain of a cytokine receptor, and, optionally,
the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R;
and, optionally, the ICD comprises a Box 1 region and a Box 2 region of a protein selected from the group consisting of G-CSFR and gp130;
and, optionally, the chimeric receptor comprises a third domain comprising at least a portion of a transmembrane domain of a protein selected from the group consisting of: G-CSFR, gp130 (Glycoprotein 130), and IL-2Rβ, and, optionally,
the transmembrane domain is a wild-type transmembrane domain; and, optionally,
the cell is an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

26. A cell comprising a nucleic acid encoding a chimeric receptor; wherein the chimeric receptor comprises: an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα; and, optionally,
the cell is an immune cell; and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

27. The cell of claim 25 or 26, wherein the ECD of the G-CSFR is encoded by nucleic acid sequence set forth in SEQ ID NO. 5 or 6.

28. The cell of claim 27, wherein the nucleic acid comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2RG having an amino acid sequence of SEQ ID NO. 17.

29. A cell comprising the expression vector of claim 17, 18 23 or 24, and, optionally,
the cell is an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

30. A cell comprising the chimeric receptor of claim 1, and, optionally,
the cell in an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

31. A cell comprising the chimeric receptor of claim 2, and, optionally,
the cell in an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

32. A method of selective activation of a chimeric receptor expressed on the surface of a cell, comprising:
contacting a chimeric receptor with a G-CSF that selectively activates the chimeric receptor; wherein the chimeric receptor comprises:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally,
the IL-2R is selected from the group consisting of IL-2Rβ and IL-2Rγc, and, optionally,
the second domain comprises at least a portion of the C-terminal region of IL-2Rβ, IL-7Rα, IL-12Rβ₂ or IL-21R; wherein
at least a portion of the ICD of the cytokine receptor comprises at least one signaling molecule binding site from an intracellular domain of a cytokine receptor, and, optionally,
the at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R, and, optionally,
the ICD comprises a Box 1 region and a Box 2 region of a protein selected from the group consisting of G-CSFR and gp130;
and, optionally, the chimeric receptor comprises a third domain comprising at least a portion of a transmembrane domain of a protein selected from the group consisting of: G-CSFR, gp130 (Glycoprotein 130), and IL-2Rβ, and, optionally,
the transmembrane domain is a wild-type transmembrane domain.

33. A method of selective activation of a chimeric receptor expressed on the surface of a cell, comprising:
contacting a chimeric receptor with a G-CSF that selectively activates the chimeric receptor;
wherein the chimeric receptor, comprises
an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

34. The method of claim 32 or 33, wherein the activated chimeric receptor forms a homodimer, and, optionally,
the activation of the chimeric receptor causes a cellular response selected from the group consisting of proliferation, viability and enhanced activity of a cell expressing the chimeric receptor; and, optionally, the chimeric receptor is activated upon contact with a G-CSF, and, optionally,
the G-CSF is a wild-type G-CSF, and, optionally,
the extracellular domain of the G-CSFR is a wild-type extracellular domain; wherein the chimeric receptor is expressed in a cell, and, optionally,
an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

35. The method of claim 34, wherein the chimeric receptor comprises
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein
the transmembrane domain comprises a sequence set forth by:
(a) SEQ ID NO. 8; or
(b) SEQ ID NO. 9; or
(c) SEQ ID NO. 10; or
(d) SEQ ID NO. 11.

36. A method of producing a chimeric receptor in an cell, comprising:
introducing into the cell the nucleic acid of any one of claims 13-16, or 19-22 or the expression vector of any one of claims claim 17, 18, 23 or 24; and, optionally, the method comprises gene editing; and, optionally,
the cell is an immune cell; and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

37. A method of treating a subject in need thereof, comprising:
infusing into the subject a cell expressing a chimeric receptor and administering a cytokine that binds the chimeric receptor; wherein the chimeric receptor comprises:
(a) an extracellular domain (ECD) of a G-CSFR (Granulocyte-Colony Stimulating Factor Receptor) operatively linked to a second domain; the second domain comprising
(b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor); and, optionally,
the IL-2R is selected from the group consisting of IL-2Rβ and IL-2Rγc; and, optionally,
the second domain comprises at least a portion of the C-terminal region of IL-2Rβ, IL-7Rα, IL-12Rβ₂ or IL-21R; wherein
at least a portion of the ICD of the cytokine receptor comprises at least one signaling molecule binding site from an intracellular domain of a cytokine receptor; and, optionally,
the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R;
and, optionally, the ICD comprises a Box 1 region and a Box 2 region of a protein selected from the group consisting of G-CSFR and gp130;
and, optionally, the chimeric receptor comprises comprising a third domain comprising at least a portion of a transmembrane domain of a protein selected from the group consisting of: G-CSFR, gp130 (Glycoprotein 130), and IL-2Rβ; and, optionally,
the transmembrane domain is a wild-type transmembrane domain.

38. A method of treating a subject in need thereof, comprising:
infusing into the subject a cell expressing a chimeric receptor and administering a cytokine that binds the chimeric receptor; wherein the chimeric receptor comprises:
an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

39. The method of claim 37 or 38, wherein the activated chimeric receptor forms a homodimer; and, optionally,
the activation of the chimeric receptor causes a cellular response selected from the group consisting of proliferation, viability and enhanced activity of a cell expressing the chimeric receptor; and, optionally, the chimeric receptor is activated upon contact with a G-CSF; and, optionally,
the G-CSF is a wild-type G-CSF; and, optionally,
the extracellular domain of the G-CSFR is a wild-type extracellular domain; wherein the chimeric receptor is expressed in a cell; and, optionally,
the cell is an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

40. The method of claim 39, wherein the chimeric receptor optionally comprises:
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein
the transmembrane domain comprises a sequence set forth by:
(a) SEQ ID NO. 8; or
(b) SEQ ID NO. 9; or
(c) SEQ ID NO. 10; or
(d) SEQ ID NO. 11.

41. The method of claim 37 or 38, wherein the method is used to treat cancer.

42. The method of claim 37 or 38, wherein the method is used to treat an autoimmune disease.

43. The method of claim 37 or 38, wherein the method is used to treat an inflammatory condition.

44. The method of claim 37 or 38, wherein the method is used to prevent or treat graft rejection.

45. The method of claim 37 or 38, wherein the method is used to treat an infection disease.

46. The method of claim 37 or 38; further comprising administering at least one additional active agent; and, optionally, the additional active agent is an additional cytokine.

47. The method of any one of claim 37, wherein the method comprises:
i) isolating an immune cell-containing sample; (ii) transducing or transfecting the immune cells with a nucleic acid sequence encoding the chimeric cytokine receptor; (iii) administering or infusing the immune cells from (ii) to the subject; and (iv) contacting the immune cells with the cytokine that binds the chimeric receptor.

48. The method of any one of claim 38, wherein the method comprises:
i) isolating an immune cell-containing sample; (ii) transducing or transfecting the immune cells with a nucleic acid sequence encoding the chimeric cytokine receptor; (iii) administering or infusing the immune cells from (ii) to the subject; and (iv) contacting the immune cells with the cytokine that binds the chimeric receptor.

49. The method of claim 47 or 48; wherein the subject has undergone an immuno-depletion treatment prior to administering or infusing the cells to the subject.

50. The method of claim 47 or 48, wherein the immune cell-containing sample is isolated from the subject that will be administered or infused with the cells.

51. The method of claim 47 or 48, wherein the immune cells are contacted with the cytokine in vitro prior to administering or infusing the cells to the subject.

52. The method of claim 46 or 47, wherein the immune cells are contacted with the cytokine that binds the chimeric receptor for a sufficient time to activate signaling from the chimeric receptor.

53. A kit for treating a subject in need thereof, comprising:
cells encoding a chimeric receptor of any one of claim 1-12,
and, optionally, the cells are immune cells; and
instructions for use;
and, optionally, the kit comprises a cytokine that binds the chimeric receptor.

54. A kit for producing a chimeric receptor expressed on a cell, comprising:
an expression vector encoding the chimeric receptor of any one of claims 1-12 and instructions for use;
and, optionally, the kit comprises a cytokine that binds the chimeric receptor.

55. A kit for producing a chimeric receptor expressed on a cell, comprising:
cells comprising an expression vector encoding the chimeric receptor of any one of claims 1-12 and, optionally,
the cells are bacterial cells, and
instructions for use;
and, optionally, the kit comprises a cytokine that binds the chimeric receptor.


SEQUENCE LISTING (80 sequences)

SEQ ID NO: 1 - protein, 24 aa, Homo sapiens
MARLGNCSLTWAALIILLLPGSLE

SEQ ID NO: 2 - protein, 22 aa, Homo sapiens
MLLLVTSLLLCELPHPAFLLIP

SEQ ID NO: 3 - DNA, 72 bp, Homo sapiens
atggcaaggctgggaaactgcagcctgacttgggctgccctgatcatcctgctgctccccggaagtctggag

SEQ ID NO: 4 - DNA, 66 bp, Mus musculus
atgctgctgctagtgacctccctgctgctctgtgagctgcctcacccggcgttcctgctgattcct

SEQ ID NO: 5 - protein, 603 aa, Homo sapiens
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 6 - DNA, 1809 bp, Homo sapiens
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 7 - DNA, 1809 bp, Artificial Sequence
  Description of Artificial Sequence Synthetic polynucleotide
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacgcatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgccccaccccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcatcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 8 - protein, 23 aa, Homo sapiens
IILGLFGLLLLLTCLCGTAWLCC

SEQ ID NO: 9 - protein, 22 aa, Homo sapiens
AIVVPVCLAFLLTTLLGVLFCF

SEQ ID NO: 10 - protein, 25 aa, Homo sapiens
IPWLGHLLVGLSGAFGFIILVYLLI

SEQ ID NO: 11 - protein, 21 aa, Homo sapiens
VVISVGSMGLIISLLCVYFWL

SEQ ID NO: 12 - DNA, 69 bp, Homo sapiens
atcatcctgggcctgttcggcctcctgctgttgctcacctgcctctgtggaactgcctggctctgttgc

SEQ ID NO: 13 - DNA, 66 bp, Homo sapiens
gccatagtcgtgcctgtttgcttagcattcctattgacaactcttctgggagtgctgttctgcttt

SEQ ID NO: 14 - DNA, 75 bp, Homo sapiens
attccgtggctcggccacctcctcgtgggtctcagcggggcttttggcttcatcatcttagtgtacttgctgatc

SEQ ID NO: 15 - DNA, 63 bp, Homo sapiens
gtggttatctctgttggctccatgggattgattatcagccttctctgtgtgtatttctggctg

SEQ ID NO: 16 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 17 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 18 - protein, 101 aa, Homo sapiens
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSIS

SEQ ID NO: 19 - protein, 205 aa, Homo sapiens
ASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 20 - protein, 62 aa, Homo sapiens
SPNRKNPLWPSVPDPAHSSLGSWVPTIMEEDAFQLPGLGTPPITKLTVLEEDEKKPVPWESH

SEQ ID NO: 21 - protein, 202 aa, Homo sapiens
SSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 22 - protein, 67 aa, Homo sapiens
SPNRKNPLWPSVPDPAHSSLGSWVPTIMEEDAFQLPGLGTPPITKLTVLEEDEKKPVPWESHNSSET

SEQ ID NO: 23 - protein, 72 aa, Homo sapiens
AGDLPTHDGYLPSNIDDLPSHEAPLADSLEELEPQHISLSVFPSSSLHPLTFSCGDKLTLDQLKMRCDSLML

SEQ ID NO: 24 - protein, 67 aa, Homo sapiens; identical to SEQ ID NO: 22

SEQ ID NO: 25 - protein, 30 aa, Homo sapiens
SPGDEGPPRSYLRQWVVIPPPLSSPGPQAS

SEQ ID NO: 26 - protein, 63 aa, Homo sapiens
SPNRKNPLWPSVPDPAHSSLGSWVPTIMEEDAFQLPGLGTPPITKLTVLEEDEKKPVPWESHN

SEQ ID NO: 27 - protein, 183 aa, Homo sapiens
QNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 28 - protein, 93 aa, Homo sapiens
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQ

SEQ ID NO: 29 - protein, 202 aa, Homo sapiens; identical to SEQ ID NO: 21

SEQ ID NO: 30 - protein, 62 aa, Homo sapiens; identical to SEQ ID NO: 20

SEQ ID NO: 31 - protein, 43 aa, Homo sapiens
SSNHSLTSCFTNQGYFFFHLPDALEIEACQVYFTYDPYSEEDP

SEQ ID NO: 32 - protein, 20 aa, Homo sapiens
AGDLPTHDGYLPSNIDDLPS

SEQ ID NO: 33 - protein, 116 aa, Homo sapiens
GGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 34 - protein, 93 aa, Homo sapiens; identical to SEQ ID NO: 28

SEQ ID NO: 35 - protein, 43 aa, Homo sapiens; identical to SEQ ID NO: 31

SEQ ID NO: 36 - protein, 20 aa, Homo sapiens
AGDLPTHDGYLPSNIDDLPS

SEQ ID NO: 37 - protein, 116 aa, Homo sapiens; identical to SEQ ID NO: 33

SEQ ID NO: 38 - protein, 138 aa, Homo sapiens
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSR

SEQ ID NO: 39 - protein, 205 aa, Homo sapiens; identical to SEQ ID NO: 19

SEQ ID NO: 40 - protein, 62 aa, Homo sapiens; identical to SEQ ID NO: 20

SEQ ID NO: 41 - protein, 67 aa, Homo sapiens
SGKNGPHVYQDLLLSLGTTNSTLPPPFSLQSGILTLNPVAQGQPILTSLGSNQEEAYVTMSSFYQNQ

SEQ ID NO: 42 - protein, 93 aa, Homo sapiens; identical to SEQ ID NO: 28

SEQ ID NO: 43 - protein, 77 aa, Homo sapiens
SSSRSLDCRESGKNGPHVYQDLLLSLGTTNSTLPPPFSLQSGILTLNPVAQGQPILTSLGSNQEEAYVTMSSFYQNQ

SEQ ID NO: 44 - DNA, 861 bp, Homo sapiens
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 45 - DNA, 261 bp, Homo sapiens
gaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 46 - DNA, 303 bp, Homo sapiens
aataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttct

SEQ ID NO: 47 - DNA, 618 bp, Homo sapiens
gcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 48 - DNA, 186 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccat

SEQ ID NO: 49 - DNA, 609 bp, Homo sapiens
agcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 50 - DNA, 201 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacc

SEQ ID NO: 51 - DNA, 219 bp, Homo sapiens
gcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 52 - DNA, 201 bp, Homo sapiens; identical to SEQ ID NO: 50

SEQ ID NO: 53 - DNA, 93 bp, Artificial Sequence
  Description of Artificial Sequence Synthetic oligonucleotide
agccctggggacgaaggacccccccggagctacctccgccagtgggtggtcattcctccgccactttcgagccctggaccccaggccagctaa

SEQ ID NO: 54 - DNA, 189 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataac

SEQ ID NO: 55 - DNA, 549 bp, Artificial Sequence
  Description of Artificial Sequence Synthetic polynucleotide
cagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccctggggacgaaggacccccccggagctacctccgccagtgggtggtcattcctccgccactttcgagccctggaccccaggccagc

SEQ ID NO: 56 - DNA, 279 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccag

SEQ ID NO: 57 - DNA, 609 bp, Homo sapiens; identical to SEQ ID NO: 49

SEQ ID NO: 58 - DNA, 186 bp, Homo sapiens; identical to SEQ ID NO: 48

SEQ ID NO: 59 - DNA, 129 bp, Homo sapiens
agcagcaaccactcgctgaccagctgcttcaccaaccagggttacttcttcttccacctcccggatgccttggagatagaggcctgccaggtgtactttacttacgacccctactcagaggaagaccct

SEQ ID NO: 60 - DNA, 60 bp, Homo sapiens
gcaggtgaccttcccacccatgatggctacttaccctccaacatagatgacctcccctca

SEQ ID NO: 61 - DNA, 351 bp, Homo sapiens
ggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 62 - DNA, 279 bp, Homo sapiens; identical to SEQ ID NO: 56

SEQ ID NO: 63 - DNA, 129 bp, Homo sapiens; identical to SEQ ID NO: 59

SEQ ID NO: 64 - DNA, 60 bp, Homo sapiens; identical to SEQ ID NO: 60

SEQ ID NO: 65 - DNA, 351 bp, Homo sapiens; identical to SEQ ID NO: 61

SEQ ID NO: 66 - DNA, 414 bp, Artificial Sequence
  Description of Artificial Sequence Synthetic polynucleotide
aataagcgagacctaattaaaaaacacatctggcctaatgttccagatccttcaaagagtcatattgcccagtggtcacctcacactcctccaaggcacaatttcaattcaaaggatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttctagcagtgatgaaaatgaatcttcacaaaacacttcgagcactgtccagtattctaccgtggtacacagtggctacagacaccaagttccgtcagtccaagtcttctcaaga

SEQ ID NO: 67 - DNA, 618 bp, Artificial Sequence; identical to SEQ ID NO: 47
  Description of Artificial Sequence Synthetic polynucleotide

SEQ ID NO: 68 - DNA, 186 bp, Homo sapiens; identical to SEQ ID NO: 48

SEQ ID NO: 69 - DNA, 204 bp, Homo sapiens
agtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 70 - DNA, 279 bp, Homo sapiens; identical to SEQ ID NO: 56

SEQ ID NO: 71 - DNA, 234 bp, Homo sapiens
tcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 72 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 73 - DNA, 1405 bp, Homo sapiens
agcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgaggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctcca

SEQ ID NO: 74 - protein, 9 aa, Homo sapiens
LWPSVPDPA

SEQ ID NO: 75 - protein, 9 aa, Homo sapiens
IWPNVPDPS

SEQ ID NO: 76 - protein, 9 aa, Homo sapiens
LKCNTPDPS

SEQ ID NO: 77 - protein, 9 aa, Homo sapiens
KIWAVPSPE

SEQ ID NO: 78 - protein, 9 aa, Homo sapiens
CSREIPDPA

SEQ ID NO: 79 - protein, 9 aa, Homo sapiens
VWPSLPDHK

SEQ ID NO: 80 - protein, 175 aa, Artificial Sequence
  Description of Artificial Sequence Synthetic polypeptide
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP